SEQUENCE LISTING (8 sequences)

SEQ ID NO: 1 - DNA, 11627 bp, synthetic construct
  source 1..11627: mol_type = other DNA
ggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaatactcgaatggtgcaaaacctttcgcggtatggcatgatagcgccgaattcgaaagaggagaaaggagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacccgggctcggtaccaaattccagaaaagaggcctcccgaaaggggggccttttttcgttttggtccgagctccaattccgacgtctaagaaaccattattatcatgacattaacctataaaaataggcgtatcacgaggccctttcgtcttcacctcgagtccctatcagtgatagagattgacatccctatcagtgatagagatactgagcacatcagcaggacgcactgaccccatggtctagagaaagaggagaaaggagatatggagacccgattccctcagcaatcgcagcaaactccggcatctaccaacagacgccggccattcaagcatgaggattacccatgtcgaagacaacaaagaagttcaactctttatgtattgatcttcctcgcgatctttctctcgaaatttaccaatcaattgcttctgtcgctactggaagcggtgatccgcacagtgacgactttacagcaattgcttacttaaggtaaagaggggaaaggagatatggtacgctggactttgtgggataccctcgctttcctcctgttgctcagtttattgctgccgtcattgctgatcatgttcatcccgtcaacattcaaacggcctgtctcatcatggaaggcgctgaatttacggaaaacactgttaatggcgtcgagcgtccggctgaagccgctgaattgttcgcgtttaccttgcgtgtacgcgcaggaaacactgacgttcttactgacgcagaagaaaacgtgcgtcaaaaattacgtgcggaaggagtgatgtaatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagataactagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggctttgcatgcgttttcctgcctcattttctgcaaaccgcgccattcccggcgcggtctgagcgtgtcagtgcaactgcattaaaaccgccccgcaaagcgggcg

SEQ ID NO: 2 - DNA, 9667 bp, synthetic construct
  source 1..9667: mol_type = other DNA
ggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaatactcgaatggtgcaaaacctttcgcggtatggcatgatagcgccgaattcgaaagaggagaaaggagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacccgggctcggtaccaaattccagaaaagaggcctcccgaaaggggggccttttttcgttttggtccctagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggctttgcatgcgttttcctgcctcattttctgcaaaccgcgccattcccggcgcggtctgagcgtgtcagtgcaactgcattaaaaccgccccgcaaagcgggcg

SEQ ID NO: 3 - DNA, 11626 bp, synthetic construct
  source 1..11626: mol_type = other DNA
ggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaatactcgaatggtgcaaaacctttcgcggtatggcatgatagcgccgaattcgaaagaggagaaaggagatcaatgaaaaaggccctggcaacactgattgcactggcactgccggcagcagcactggcagaaggtaccgaaaacgtgcagtatcgccacgttgaactggcacgcgttggccaattagttgaggtggacaccctggaacacgtgcagcatattattggcggtgccggcaatgacagcattacaggcaacgcccacgacaactttctggcaggcggcagcggcgatgatcgtctggatggtggcgccggtaatgataccctggtgggtggcgagggtcaaaataccgttattggcggtgccggcgacgatgttttcctgcaggatctgggcgtgtggagcaaccagctggacggtggtgcaggtgtggacaccgtgaaatacaacgttcatcagccgagcgaggaacgtctggaacgcatgggtgacaccggtattcatgccgatctgcagaaaggcaccgtggaaaaatggccggccctgaacctgtttagcgtggatcatgttaaaaatattgaaaatctgcatggcagccgtctgaacgaccgcattgccggcgatgaccaagataacgaactgtggggccatgacggtaatgacaccattcgcggccgtggtggcgacgacattctgcgtggtggtctgggtctggatacactgtacggtgaggatggcaatgatatctttctgcaggatgacgaaacagttagcgacgacattgacggtggcgcaggtctggacacagtggactacagcgccatgattcatccgggccgtattgtggccccgcacgaatatggttttggtatcgaagccgatctgagccgtgaatgggtgcgcaaagcaagtgccctgggtgtggactattacgacaacgtgcgcaatgtggaaaacgtgattggcaccagcatgaaagacgtgctgattggcgacgcccaggccaacaccctgatgggccagggtggtgacgatacagtgcgcggtggtgatggtgatgacctgctgttcggcggcgatggtaacgacatgctgtatggcgatgccggtaacgacaccctgtacggcggtctgggtgacgataccctggagggcggtgccggcaacgattggtttggccaaacccaggcccgtgaacatgatgtgctgcgcggtggcgatggcgtggataccgttgactacagtcagacaggcgcccatgcaggtatcgcagccggtcgcattggtctgggtattctggcagacctgggtgccggtcgtgtggataaactgggtgaggccggcagtagtgcctatgacacagtgagcggcatcgaaaacgtggtgggcacagagctggcagaccgtatcacaggcgatgcccaagccaatgttctgcgcggcgcaggtggtgccgatgtgttagccggtggtgagggtgatgacgttctgctgggcggtgacggcgatgatcagctgagtggtgatgcaggtcgcgaccgcctgtatggcgaagcaggcgatgattggttctttcaggacgcagccaatgccggtaatctgttagacggtggcgacggtcgtgatacagtggattttagcggtcctggtcgtggcctggatgccggcgcaaagggtgtgtttctgagcctgggcaaaggctttgccagcttaatggacgaaccggaaaccagcaacgtgttacgcaacattgagaacgccgtgggtagtgcccgcgatgacgtgctgatcggtgatgccggtgcaaatgttctgaacggcctggccggcaacgacgtgctgagtggtggtgcaggtgatgatgttctgctgggtgatgagggcagtgacttactgagcggtgacgccggcaatgacgatttattcggcggccagggtgatgacacctacctgtttggcgtgggttatggccacgacaccatttatgagagcggtggtggtcacgacacaatccgcatcaatgcaggcgccgatcagctgtggtttgcccgccagggtaacgatctggagattcgtatcctgggcacagacgatgccttaacagtgcatgattggtaccgcgatgccgatcatcgcgtggagattatccatgcagcaaaccaggcagtggatcaggccggcatcgagaagctggttgaggccatggcccagtatcctgatcctggtggtcatcatcaccaccatcattaataactcgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacccgggctcggtaccaaattccagaaaagaggcctcccgaaaggggggccttttttcgttttggtccctagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggctttgcatgcgttttcctgcctcattttctgcaaaccgcgccattcccggcgcggtctgagcgtgtcagtgcaactgcattaaaaccgccccgcaaagcgggcg

SEQ ID NO: 4 - DNA, 11624 bp, synthetic construct
  source 1..11624: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaataccaattccgacgtctaagaaaccattattatcatgacattaacctataaaaataggcgtatcacgaggccctttcgtcttcacctcgagtccctatcagtgatagagattgacatccctatcagtgatagagatactgagcacatcagcaggacgcactgaccgaattcgaaagaggagaaaggagatcatatgaaaaaggccctggcaacactgattgcactggcactgccggcagcagcactggcagaaggtaccgaaaacgtgcagtatcgccacgttgaactggcacgcgttggccaattagttgaggtggacaccctggaacacgtgcagcatattattggcggtgccggcaatgacagcattacaggcaacgcccacgacaactttctggcaggcggcagcggcgatgatcgtctggatggtggcgccggtaatgataccctggtgggtggcgagggtcaaaataccgttattggcggtgccggcgacgatgttttcctgcaggatctgggcgtgtggagcaaccagctggacggtggtgcaggtgtggacaccgtgaaatacaacgttcatcagccgagcgaggaacgtctggaacgcatgggtgacaccggtattcatgccgatctgcagaaaggcaccgtggaaaaatggccggccctgaacctgtttagcgtggatcatgttaaaaatattgaaaatctgcatggcagccgtctgaacgaccgcattgccggcgatgaccaagataacgaactgtggggccatgacggtaatgacaccattcgcggccgtggtggcgacgacattctgcgtggtggtctgggtctggatacactgtacggtgaggatggcaatgatatctttctgcaggatgacgaaacagttagcgacgacattgacggtggcgcaggtctggacacagtggactacagcgccatgattcatccgggccgtattgtggccccgcacgaatatggttttggtatcgaagccgatctgagccgtgaatgggtgcgcaaagcaagtgccctgggtgtggactattacgacaacgtgcgcaatgtggaaaacgtgattggcaccagcatgaaagacgtgctgattggcgacgcccaggccaacaccctgatgggccagggtggtgacgatacagtgcgcggtggtgatggtgatgacctgctgttcggcggcgatggtaacgacatgctgtatggcgatgccggtaacgacaccctgtacggcggtctgggtgacgataccctggagggcggtgccggcaacgattggtttggccaaacccaggcccgtgaacatgatgtgctgcgcggtggcgatggcgtggataccgttgactacagtcagacaggcgcccatgcaggtatcgcagccggtcgcattggtctgggtattctggcagacctgggtgccggtcgtgtggataaactgggtgaggccggcagtagtgcctatgacacagtgagcggcatcgaaaacgtggtgggcacagagctggcagaccgtatcacaggcgatgcccaagccaatgttctgcgcggcgcaggtggtgccgatgtgttagccggtggtgagggtgatgacgttctgctgggcggtgacggcgatgatcagctgagtggtgatgcaggtcgcgaccgcctgtatggcgaagcaggcgatgattggttctttcaggacgcagccaatgccggtaatctgttagacggtggcgacggtcgtgatacagtggattttagcggtcctggtcgtggcctggatgccggcgcaaagggtgtgtttctgagcctgggcaaaggctttgccagcttaatggacgaaccggaaaccagcaacgtgttacgcaacattgagaacgccgtgggtagtgcccgcgatgacgtgctgatcggtgatgccggtgcaaatgttctgaacggcctggccggcaacgacgtgctgagtggtggtgcaggtgatgatgttctgctgggtgatgagggcagtgacttactgagcggtgacgccggcaatgacgatttattcggcggccagggtgatgacacctacctgtttggcgtgggttatggccacgacaccatttatgagagcggtggtggtcacgacacaatccgcatcaatgcaggcgccgatcagctgtggtttgcccgccagggtaacgatctggagattcgtatcctgggcacagacgatgccttaacagtgcatgattggtaccgcgatgccgatcatcgcgtggagattatccatgcagcaaaccaggcagtggatcaggccggcatcgagaagctggttgaggccatggcccagtatcctgatcctggtggtcatcatcaccaccatcattaataactcgagtaacccgggctcggtaccaaattccagaaaagaggcctcccgaaaggggggccttttttcgttttggtccgagctctggtaaatagaaacggaactttacatattgaataccggagatataattcgtttatgccacataatcatcaatacatccgtaacccgcccgaaacggaaaaactgaagaaaaaccccattatcttagcctaactagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggcttt

SEQ ID NO: 5 - DNA, 11624 bp, synthetic construct
  source 1..11624: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaatactcgaatggtgcaaaacctttcgcggtatggcatgatagcgccgaattcgaaagaggagaaaggagatcatatgctctccacactccgccgcactctatttgcgctgctggcttgtgcgtcttttatcgtccatgccgatccgccggccaccgtgtataaatatgatagccgcccgccggaagatgtgtttcagaatggcttcaccgcctggggcaacaatgataacgtgctggaccacctgaccggtcgtagcggtcaggtgggtagcagcaacagtgcctttgtgagcaccagcagcagccgtcgctataccgaagtgtacctggaacatcgcatgcaggaagcagtggaagcagaacgtgcaggccgtggcaccggccattttattggctacatttatgaggtgcgcgccgataataacttttacggcgccgccagcagctactttgaatacgtggacacctacggcgacaatgccggtcgtatcctggcaggtgccctggcaacctatcagagcggttatctggcccatcgccgcattccgccggaaaatattcgccgtgtgacccgcgtgtatcataacggcattaccggcgaaaccaccaccaccgagtatagcaatgcccgctatgttagccagcagacccgcgccaatccgaatccgtataccagcggcggtcatcatcaccaccaccattaataactcctttagcgtggataaaaaataataacccgggctcggtaccaaattccagaaaagaggcctcccgaaaggggggccttttttcgttttggtcctggtaaatagaaacggaactttacatattgaataccggagatataattcgtttatgccacataatcatcaatacatccgtaacccgcccgaaacggaaaaactgaagaaaaaccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaaactagacccgccgccaccatggagatgagtgacgcggcaccgagcttgtctaaccttttctatgatccgacctacaaccctgggcaaagtacgattgattacaaagacgatgacgacaaggccgagatctataataaagacggaaataaattggatttgttcggtaaagtcgacggacttcactacttctctgatgacaagggaagtgacggagaccagacttacatgagaatcggattcaagggtgagacccaagttaacgaccagcttaccggatacggtcagtgggagtaccagatccaaggaaaccagaccgagggtagtaacgactcttggaccagagtcgctttcgccggtttgaagttcgctgacgctggtagtttcgactacggaaggaattacggagtcacctacgacgttacctcttggaccgacgtccttccagaattcggtggagatacctacggtgccgacaacttcatgcagcaaaggggtaacggttatgccacctatagaaacaccgacttcttcggtttggtcgacggtttggacttcgccttgcagtaccaaggtaagaatggtagtgtttctggtgaaaataccaatggtagatctttgcttaatcagaacggagacggttacggtggttctttgacctacgccatcggtgaaggtttctctgtcggaggtgccattaccaccagtaagagaaccgccgaccagaacaataccgccaacgccagattgtacggaaacggtgatagagccaccgtctacaccggtggtttgaaatacgacgctaacaacatctaccttgctgcccagtacagtcagacctacaatgccactagattcggaaccagtaacggtagtaacccaagtacctcttacggatttgccaacaaggcccagaactttgaggtcgttgcccagtaccagttcgacttcggtcttaggccaagtgtcgcttatcttcagtctaagggtaaggacatctctaacggttacggagcctcttacggtgaccaagatatcgtcaagtacgtcgacgtcggagctacctactactttaataaaaacatgagtacttacgtcgattataagatcaatttgttggacaaaaacgacttcactagagatgccggtatcaatctagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggcttt

SEQ ID NO: 6 - DNA, 11624 bp, synthetic construct
  source 1..11624: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtccctatcagtgatagagattgacatccctatcagtgatagagatactgagcacatcagcaggacgcactgaccgaattcgaaagaggagaaaggagatcatatgctctccacactccgccgcactctatttgcgctgctggcttgtgcgtcttttatcgtccatgccgatccgccggccaccgtgtataaatatgatagccgcccgccggaagatgtgtttcagaatggcttcaccgcctggggcaacaatgataacgtgctggaccacctgaccggtcgtagcggtcaggtgggtagcagcaacagtgcctttgtgagcaccagcagcagccgtcgctataccgaagtgtacctggaacatcgcatgcaggaagcagtggaagcagaacgtgcaggccgtggcaccggccattttattggctacatttatgaggtgcgcgccgataataacttttacggcgccgccagcagctactttgaatacgtggacacctacggcgacaatgccggtcgtatcctggcaggtgccctggcaacctatcagagcggttatctggcccatcgccgcattccgccggaaaatattcgccgtgtgacccgcgtgtatcataacggcattaccggcgaaaccaccaccaccgagtatagcaatgcccgctatgttagccagcagacccgcgccaatccgaatccgtataccagcggcggtcatcatcaccaccaccattaataactcctttagcgtggataaaaaataataacccgggctcggtaccaaattccagaaaagaggcctcccgaaaggggggccttttttcgttttggtcctggtaaatagaaacggaactttacatattgaataccggagatataattcgtttatgccacataatcatcaatacatccgtaacccgcccgaaacggaaaaactgaagaaaaaccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaaactagacccgccgccaccatggagatgagtgacgcggcaccgagcttgtctaaccttttctatgatccgacctacaaccctgggcaaagtacgattgattacaaagacgatgacgacaaggccgagatctataataaagacggaaataaattggatttgttcggtaaagtcgacggacttcactacttctctgatgacaagggaagtgacggagaccagacttacatgagaatcggattcaagggtgagacccaagttaacgaccagcttaccggatacggtcagtgggagtaccagatccaaggaaaccagaccgagggtagtaacgactcttggaccagagtcgctttcgccggtttgaagttcgctgacgctggtagtttcgactacggaaggaattacggagtcacctacgacgttacctcttggaccgacgtccttccagaattcggtggagatacctacggtgccgacaacttcatgcagcaaaggggtaacggttatgccacctatagaaacaccgacttcttcggtttggtcgacggtttggacttcgccttgcagtaccaaggtaagaatggtagtgtttctggtgaaaataccaatggtagatctttgcttaatcagaacggagacggttacggtggttctttgacctacgccatcggtgaaggtttctctgtcggaggtgccattaccaccagtaagagaaccgccgaccagaacaataccgccaacgccagattgtacggaaacggtgatagagccaccgtctacaccggtggtttgaaatacgacgctaacaacatctaccttgctgcccagtacagtcagacctacaatgccactagattcggaaccagtaacggtagtaacccaagtacctcttacggatttgccaacaaggcccagaactttgaggtcgttgcccagtaccagttcgacttcggtcttaggccaagtgtcgcttatcttcagtctaagggtaaggacatctctaacggttacggagcctcttacggtgaccaagatatcgtcaagtacgtcgacgtcggagctacctactactttaataaaaacatgagtacttacgtcgattataagatcaatttgttggacaaaaacgacttcactagagatgccggtatcaatctagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggcttt

SEQ ID NO: 7 - DNA, 11624 bp, synthetic construct
  source 1..11624: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtgcgttcctgtatttccggagggccctaatactcgaatggtgcaaaacctttcgcggtatggcatgatagcgccgaattcgaaagaggagaaaggagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacccgggctcggtaccaaattccagaaaagaggcctcccgaaaggggggccttttttcgttttggtccgagctctggtaaatagaaacggaactttacatattgaataccggagatataattcgtttatgccacataatcatcaatacatccgtaacccgcccgaaacggaaaaactgaagaaaaaccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttactagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggcttt

SEQ ID NO: 8 - DNA, 11624 bp, synthetic construct
  source 1..11624: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattcgtggcatgagagagttaaagggcgtccctatcagtgatagagattgacatccctatcagtgatagagatactgagcacatcagcaggacgcactgaccgaattcgaaagaggagaaaggagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacccgggctcggtaccaaattccagaaaagaggcctcccgaaaggggggccttttttcgttttggtccgagctctggtaaatagaaacggaactttacatattgaataccggagatataattcgtttatgccacataatcatcaatacatccgtaacccgcccgaaacggaaaaactgaagaaaaaccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttactagcataaccccttggggcctctaaacgggtcttgaggggttttttgagacaaacaaaagaatggaatcaaagttaatgctagcagccgccgctgcaggcatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatttgtgtctcaaaatctctgatgttacattgcacaagataaaaatatatcatcatgaacaataaaactgtctgcttacataaacagtaatacaaggggtgttatgagccatattcagcgtgaaacgagctgtagccgtccgcgtctgaacagcaacatggatgcggatctgtatggctataaatgggcgcgtgataacgtgggtcagagcggcgcgaccatttatcgtctgtatggcaaaccggatgcgccggaactgtttctgaaacatggcaaaggcagcgtggcgaacgatgtgaccgatgaaatggtgcgtctgaactggctgaccgaatttatgccgctgccgaccattaaacattttattcgcaccccggatgatgcgtggctgctgaccaccgcgattccgggcaaaaccgcgtttcaggtgctggaagaatatccggatagcggcgaaaacattgtggatgcgctggccgtgtttctgcgtcgtctgcatagcattccggtgtgcaactgcccgtttaacagcgatcgtgtgtttcgtctggcccaggcgcagagccgtatgaacaacggcctggtggatgcgagcgattttgatgatgaacgtaacggctggccggtggaacaggtgtggaaagaaatgcataaactgctgccgtttagcccggatagcgtggtgacccacggcgattttagcctggataacctgattttcgatgaaggcaaactgattggctgcattgatgtgggccgtgtgggcattgcggatcgttatcaggatctggccattctgtggaactgcctgggcgaatttagcccgagcctgcaaaaacgtctgtttcagaaatatggcattgataatccggatatgaacaaactgcaatttcatctgatgctggatgaatttttctaacttggaagtaagaatggtgccgaaggccggactcaaacatcaaaataagttaatgataaaaaacaaataataaaacacaacaatgaaatatgcccccttttgtgcccccactgtttttctgaccaatctattttcagcccatcaataaatcggaaagttaaatcatttttaatcagtaagtttggatccgtagctcggatccaaaccagtgcatcttttatccacataaaaaattttttttcgaaagaactgttcacactgttcacctttctgttttctccttttatttcagagtgataggtggtgaataatgggtgaagggtgaacattcgattcttcacctccggcattctgccgatgtgactcataccggtgattaatcctccgcactgaaatcactcaggaagaaaaaagttttttttgatttgattgttcacactgttcacctttcgtttttctcttttaatttcagtgtgataacgggtgaatatacggtgaagggtgaacagtggattgttcaccttcgggggatatcgggataaaaaaagaccggcagatgccggtcaggtgggtcaggctgttgtagggtcgtcacattttggcagccagtcgccgtagctttcctctttcagcgtcaggttggtctgtatcccctgtttggtatggcgtttctcgtaattcagtccgtattccttcagcatcaccggcagccccagcccgaacattttcagactgagtacattccggtagccgtttgcctccatgtaggccagataggcgtgatagaggtatttacggtaattgcgcgggatgatactggcgttccccatatacatgccgctggtctgcggcagggtttccagatagccgataaaatcaaacgtcgggtcggcatcccgtttgatgttcagtgcctcgtctgagttctgctgggactgaagcagtgaccgggcgagcatcgggtcgctgaacttctgcatcaggtgacgcacgatgaccgccagctcgcgggtgattttgtccttaagctgcgggtcgcgctcctgcggggctatctgttccgggaagtgaataatcacccgtcggcgtgacacgccgccgctgcggtcggtgaagcgcatcgggttattgttcacggccagaatcaccgccgggatgtgcgtggagtacgcatcccggtatttcgggtcaacggacaccgcatcgccgccggtgatggccttgagtccggcaccgtcgccgctccatttttcctggtccggcaggcgtatcagtgagaagccagttaacgcggcacgttcacgcggggattccagcgtctcgatggtggccgacgtggcgttatcctccccggccagcagggtggctatttcggccatgatacttttgccgctgccgccgggaccggtcacctccagaaagagctgccagtcgtagcggtttgccagcaccataaacagtgcagccagaatcacgtcgcgtttttccgcacggccaccggcggcacggtcaagccagcgccagaaggcgggggcgtgggtttccagcgtttcaccgtccaccggcggggtgaaatccacatcgcacagggtgcgcatccagtgtgacggactgtgcgggtggaacgtgccgttctgcgtgtcgagcacgccgttacgaaagccaatcaggcggcgggagggggcttcctgctgcggaataatcagcttcagggtatccaccacggaggccaccttcccggaggagaacggcgcacgcagacgctgaaacagcccggccacatcccgggcaaagtcctgtggcggcagcaccttccagacaccattttcatagcgggacagaagctggccgttggcatcgaccgcgagcgcctcgccgtaatgctcatagatacgcatggccttttcgctggtactcatggcggaaaactccgcttcgctcatggtgtcgaacgggctttcagccggtggccggatggcatcgtaaatggccttacgggtggcctccccgccgtactgcgtgaaggcatcattccagtcaccgaagaccggcggcagggcaacaacaccttcacacgcatctgcggctgcggcggcttttttctggccgtcaccactgaggtcacggtcagcggcaaggacaatctgacaggcggggtgcttctgccgggcaaggctggccagagaaaggaggttcacggaagaaagcgccaccatcaccgtttcaccggtcaggtgatgtacggtaagtgcggtcgcgtatccctccgctatccacagacgttttccggcctgattctgtccttcaagggtgtgacaggtgcccctgacctgtccgcctttcagggtgcgcttacggccgtcagcactgattaactgaaggttaaccagttcgccgctgtcgtcatacagtggcaccacaaggtcaccggcgcgccagctcacgccaccggctctgtgtgtgccggtcagcatccggcattcccggccgggaaagcccttgcgggtcaggtaggcgttaccggttccggtacgggttttcgccatcagggtttgtgccagtgcggcggcgttcttccgggcagcgtctgtttcatcaacggcggcggtcgtcactgccgggtcagccggtggcaggctgccggtcacggcagccacctttgcggccgcgtcggacggggaaacaccaaaaaccttttcaaccagtttcaggccgtcaccggcaccacactgattgcagtaccaggtgccgcgcccctccctgtcatcaaaacggaagcggtcactcccgccacagaccggacagggctgatgacggttcttcagcacctgaatccccagcgccgggagaatacgcggccagtggccgagcgcatggctgacggtggcggttacgttcattttcatggtgttgttctccttcagtgcagtaccggcgcttttatgtgacgggcacagagttcatccatcacaaccagcccgagaaaggacagcgacggcgcggccttcagggggccggattccattaaatcttccagcagggcacaggctatctgacgccctttttcctcaccgtgctggcgcagataaaagccttccagctcagcggcgatggccgcctccagtgactcaagggtgagatgcgggtagcggtgctgacgttcgcacacggtcagccaggcacaggcgacagcgcgacggtaaagggcagcgcgtaagacgggcggtaagggtgttttcatttgcttttctccctgtgacagatgactgcattccgtgccggttgcattaactgataaggcatatctgcgtctcctgaagacgtgcgtatccctgcgcgaatacgcacatttaatttttcgggggtcgttttttaattacagataattgcggtaactgttatccggggtggtttccgggtcaggctccgtgcggggaatttcccgccattcccgcgccaccggtgctgcccggctgaccggaacagtgtcctgcgggtaaatatccagatatttttcccgccatttctgtaattccgggtctccggccatttctttcagtaccgcatgccggtttacggggctgcgtttaaacaggtcaggacggtcacaggtaaattcccgcagaaaacgccccagcgggatgtctgtggtgcgtccgtcagcgaggatacgcacaaggatactgaatttacggcggtacgggttccagacaatgtccgggcagcggtacggcatttcccacggaataccgtcttccagaatgccgaccacggccacatcgggaaaaccggcagaacggtaaatctcaccgggctggggaaaatcaaacatgcgtcctgtctccccggtctttctgctgggcgagaaaatcgcggcacaggcctttggctttcagctcattcagcacaaaatcaatatcttcattcaggtagctgaaaatatgtggaatgtagagctgatgcaggccggagagttcacggtgaatcaaatcacccccaacaaaccgggatacggcgctggcgcggttgagcttatggtaagcctcaatgctgaggtgttcacgggcgtcatgacgcgctgagacggtctgaggggcttttttattacgcacgggacacctccaccaccggcagacgggcagcaagggagagcacatagtcacggacaagggaacggcgggcactgcgttcatcaccggcgacggtgcgaagcatacagatacggggatgacggtctgcgcgacggacagccgcaaacacaaagacaaattcagggtgtgagggggtaagggttgtagccatgatggcagcctcctgtgaatagcaaataacgctatcgccggagttctcacgctcgatggcgatagcccagacgggggtgagaataccggcttcacaggataccggccagcccggaggctgccccgcctgagctaccattgactctgcggcataatgagcggacgcgggcaggatgcacggaatgccatctgcacgactgaccacacaccacaccataatctggcgctctgtggcattgattgcgacacaaaaaaagacgcgtggcgcgtcatatgtcgcctgtgaattgctcgggttctcacgcccggctgccgattttgcggcaggcgaaaaactatatccgcaaatgccggaaaaaggcaagccagaaaaagggagtttttgcagagcgggcatcatcatgcgtcgtacccccgtttgcgtccggcaatgcgtccggccatccatgcggtgacttcagagtgcagccaggccacatttttaccgccaagactcacctgcggcggaaattcccccttacggatgagttcgtagatggtcgagcgtgacaggccgcacaggtgcatcacttccggcacacgtaaaaaacgctcctgcgtgatgtccggcagcggcatcagtggcgtcactggggcgggagacggggaagaaaaaacagcttgcatcgggctacctcgttaatgtccatacagcaccggataagtccgtccggcttcgggtagcgctttattttgtgaatattttcagcagacgcaacaggggggatttgttcaggctgtcttacaatggctgtgtgttttttgttcatctccacttaaagtcatttaaagccacttaaagcaatttgtaatttttatagtgaaatacaaatcgtttcttcttattcattcccggcgaattaataaaaacaaacagtagtaaacagcacaaaaagcccatcaacgggtgaacagtggtgaacagacggtgaacagtcattactgcgattgttcaccctttaacttactgtattacttatcttttttattaaggtgaacagaggtgaacagtaaaatataaaaaaacaaacagtaagccggtttttcctgcgaccttttcctggcttgccggtctgaggatgagtctcctgtgtcagggctggcacatctgcaatgcgtcgtgttgttgtccggtgtacgtcacaattttcttaacctgaagtgacgaggagccggaaaatgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatgagggctgccggttaacaccggccgtcgccgcacaccgtatttttattcttcagcatgatgagaaagagataacgatggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaaaattccccgaaaatccgcccgtttttactgaaaaaagccatgcatcgataaggtgcatggcttt

(19) United States
(12) Patent Application Publication
(10) Pub. No.: US 2025/0249085 A1
(43) Pub. Date: Aug. 7, 2025

(54) BACTERIOPHAGE

(71) Applicant: GlaxoSmithKline Biologicals SA, Rixensart (BE)

(72) Inventors: Maria ABABI, Warwickshire (GB); Sandra GIANNINI, Rixensart (BE); Alfonso JARAMILLO ROSALES, Warwickshire (GB); Matthew TRIDGETT, Warwickshire (GB)

(73) Assignee: GlaxoSmithKline Biologicals SA, Rixensart (BE)

(21) Appl. No.: 18/854,756

(22) PCT Filed: Apr. 19, 2023
(86) PCT No.: PCT/EP2023/060106

§ 371 Date: Oct. 7, 2024

(30) Foreign Application Priority Data
Apr. 21, 2022 (GB) 2205833.3

(51) Int. Cl. A61K 39/108; A61K 39/00; A61P 31/04; C12N 7/00; C12N 15/70

(52) U.S. Cl. CPC A61K 39/0258; A61P 31/04; C12N 7/00; C12N 15/70; A61K 2039/5256; C12N 2795/10021; C12N 2795/10022; C12N 2795/10034; C12N 2795/10051; C12N 2830/005

(57) ABSTRACT

Engineered bacteriophages comprising polynucleotides encoding heterologous proteins under the control of repressible promoters are provided. Also disclosed are processes for producing the engineered bacteriophages, pharmaceutical compositions comprising the engineered bacteriophages and therapeutic and preventive methods using the engineered bacteriophages.

### TECHNICAL FIELD

The present invention relates to the field of engineered bacteriophages containing a polynucleotide encoding heterologous proteins under the control of a promoter.

### BACKGROUND

Bacteriophages have been known for many years having been discovered by Fredrick Twart in 1915 and Felix d'Herelle in 1917. They are viruses with DNA or RNA genomes that infect and replicate within bacteria. Bacteriophages can undergo lytic or lysogenic cycles within bacteria. During the lytic cycle, the bacteriophage genetic material is injected into a bacterium, where transcription, translation and replication take place, leading to the assembly and packaging of bacteriophage proteins and nucleic acids and eventually to lysis where many bacteriophage are released, ready to infect further bacteria. Some bacteriophages can also carry out a lysogenic cycle in which the bacteriophage genetic material is incorporated into a bacterial genome.

Bacteriophages are currently being tested in clinical studies for the treatment of bacterial infections. Pathogens such as S. aureus, E. coli and P. aeruginosa are being targeted. Wright A Clin Otolaryngol (2009) 34:349, describes a controlled clinical trial of a therapeutic bacteriophage preparation for the treatment of chronic otitis due to antibiotic-resistant Pseudomonas aeruginosa. Sarker S A et al. Virology (2012) 434:222 describes the administration of an oral T4-like phage cocktail to healthy adult volunteers from Bangladesh (ClinicalTrials.govidentifier: NCT01818206).

Engineered bacteriophages have been developed for multiple bacterial targets with the objective of elimination or reduction of bacterial load. Examples include; SASP gene delivery: a novel antibacterial approach. Fairhead H Drug News Perspect. (2009): 197-203, Engineered Phagemids for Nonlytic, Targeted Antibacterial Therapies; Krom R J et al Nano Lett. (2015) 15, 4808-4813; Sequence-specific antimicrobials using efficiently delivered RNA-guided nucleases Citorik R J et al. Nature Biotechnology (2014) 32 1141, Exploiting CRISPR-Cas nucleases to produce sequence-specific antimicrobials, Bikard D. Nature Biotechnology (2014) 32 1146.

Moreover, engineered bacteriophage have also been developed as vaccines or for targeted delivery to kill cancer cells. However, such bacteriophages are not intended to infect bacteria (Therapeutic and prophylactic applications of bacteriophage components in modern medicine. Adhya S et al. Cold Spring Harb Perspect Med. (2014) 1 1., Killing cancer cells by targeted drug-carrying phage nanomedicines Bar H. et al. BMC Biotechnol. (2008) 37, Phage protein-targeted cancer nanomedicines Petrenko V A and Jayanna P K. FEBS Lett. (2014) 588:341).

With the growth of antibiotic resistance, there remains a need in the art for the development of further strategies to treat or prevent bacterial infection.

The present invention provides a more efficient engineered bacteriophage which carries a gene encoding a heterologous protein, for example a prophylactic or therapeutic (e.g. as a pharmaceutical cargo). Efficient production of the bacteriophage can be achieved by improving the stability of pharmaceutical cargo so that the correct sequence of the cargo is maintained. Optionally, a second increase of efficiency can be achieved by increasing the levels of bacteriophage produced in a method of production. The invention provides a bacteriophage in which expression of a gene encoding a heterologous protein (for example a prophylactic or therapeutic protein) is under the control of repressible promoter. This results in increased stability of the gene encoding a therapeutic protein such that the likelihood of genetic alternation is reduced. In addition, especially where expression of the therapeutic protein puts a metabolic strain on the bacteriophage production host cell, the bacteriophage can be produced at higher yield. The use of a repressible promoter is advantageous over the use of an inducible promoter since this allows the repressor to be present during “in vitro” production steps where stability and yield improvements are established but allows high level expression of a heterologous protein during “in vivo” steps in the target bacterium where the repressor is absent.

Accordingly, there is provided, an engineered bacteriophage comprising a polynucleotide encoding a heterologous protein under the control of a inducible or repressible promoter, suitably a repressible promoter.

In a second embodiment of the invention, there is provided a process for producing a bacteriophage preparation containing a step of producing the engineered bacteriophage of the invention under conditions where expression of the heterologous protein is repressed.

In a third embodiment of the invention, there is provided a process for producing a engineered bacteriophage comprising a polynucleotide containing a gene encoding a heterologous (or non-bacteriophage) protein under the control of a repressible promoter comprising the steps of i) amplifying the engineered bacteriophage in bacteria in the presence of a repressor of the repressible promoter and ii) isolating the engineered bacteriophage.

In a fourth embodiment of the invention there is provided a bacteriophage polynucleotide comprising a bacteriophage packaging sequence and a gene encoding a heterologous (or non-bacteriophage) protein under the control of a repressible promoter.

In a fifth embodiment of the invention, there is provided a use of the engineered bacteriophage of the invention or the engineered bacteriophage genome polynucleotide of the invention in the expression and release of the heterologous (or non-bacteriophage) protein from a bacterium, optionally a commensal bacterium.

In a sixth embodiment of the invention, there is provided a pharmaceutical composition comprising the engineered bacteriophage or the engineered bacteriophage genome polynucleotide of the invention.

In a seventh embodiment of the invention, there is provided a vaccine comprising the engineered bacteriophage of the invention or the engineered bacteriophage genome polynucleotide of the invention.

In an eighth embodiment of the invention, there is provided an engineered bacteriophage according to the invention or a engineered bacteriophage genomic polynucleotide according to the invention, for use in the prevention of disease, optionally infectious disease or cancer.

In a ninth embodiment of the invention, there is provided method of treatment of a disease comprising the steps of: a) administering the engineered bacteriophage according to the invention or the engineered bacteriophage genomic polynucleotide according to the invention, to a patient in need thereof such that the engineered bacteriophage or engineered bacteriophage genomic polynucleotide contacts a commensal bacterium; b) entry of the genome polynucleotide into the commensal bacterium, and c) expression and release of the heterologous protein at a sufficient level for an immune response to be elicited against the heterologous protein.

### FIGURE LEGENDS

FIG. 1. Microbiome/commensal-based therapy. A) Engineered bacteriophage (blue) mixed with commensal bacteria (orange). Engineered bacteriophage could persist and influence the bacterial community. B) Helper-free Engineered non-replicative phage-like particles (blue) deliver therapeutic cargo to commensal bacteria (orange). The non-replicative phage-like particles specifically target their bacterial host only and are expected not to persist otherwise.

FIG. 2. P4 phasmid designs containing cargo. A) pP4-p72; pP4-LacI1-p72 (11,626 bps) was built by cloning, while pP4-pLtetO1-p72 (11,624 bps) was built by Genewiz synthesis; B) pP4-p65; pP4-LacIq/pLtetO1-p65 (11,624 bps) was built by Genewiz synthesis. 1=LacIq promoter; 2=pLtetO-1 promoter.

FIG. 3. Small scale production of P4 phage-like particles encoding secretory proteins. P4-LacIq/pLtetO1-p72/p65=particles encapsulating the P4 phasmid with p72/p65 controlled by the LacIq/pLtetO1 promoter. Red dot=outlier, ROUT (Q=1%); ns=not significant, *P≤0.05.

FIG. 4. P4-based phage-like particles delivery of secretory proteins in EMG2. 1=Color Prestained Protein Standard, Broad Range (11-245 kDa); 2-3=Negative control, EMG2 only; 4-5=EMG2 transduced with P4-LacIq-p65 (MOI=10); 6-7=EMG2 transduced with P4-pLtetO1-p72 (MOI=10); 8-9=EMG2 transduced with P4-pLtetO1-p65 supernatant (MOI=10). 2× concentrated supernatant after 3 hrs and 6 hrs post-transduction for each, respectively. Detected by anti-5×his-HRP antibody (1:1000).

FIG. 5. P4-based phage-like particles delivery of large secretory proteins in EMG2. 1=Color Prestained Protein Standard, Broad Range (11-245 kDa); 2-4=EMG2 transduced with P4-LacIq-p72 (MOI=0.01); 5-7=EMG2 transduced with P4-pLtetO1-p72 (MOI=0.01); 8-10=EMG2 transduced with P4-pLtetO1-p72 (MOI=10). 20× concentrated supernatant after 3 hrs, 6 hrs and overnight post-transduction each, respectively. Detected by anti-5×his-HRP antibody (1:1000).

FIG. 6. Small scale production of P4 phage-like particles encapsulating large secretory constructs regulated by LacIq and pLtetO-1 promoters, respectively. ns=not significant, *P≤0.05.

FIG. 7. Sequencing results for LacIq regulated constructs miniprepped from transduced EMG2, mixed with P4_col1_F primer. A) pP4-LacIq-p65; B) pP4-LacIq-p72. Original sequence is on first row, with p65/p72 gene highlighted.

FIG. 8. Sequencing results for pLtetO-1 regulated constructs miniprepped from transduced EMG2. A) pP4-pLtetO1-p65, mixed with P4-col1-F primer; B) pP4-pLtetO1-p72, mixed with pP4_Del2_Rev primer; C) pP4-pLtetO1-p72, mixed with P4-col1-F primer; ABC) Original sequence is on first row, with p65/p72 gene highlighted. D) The fragment deletion from C) is highlighted on the pLtetO-1 promoter sequence.

FIG. 9. Dual-action phasmid maps. A=Lysis only control; B=Lysis and antigen; packs inside P4 capsids; 1=LacIq; 2=LacI, weaker variant of LacIq; 3=tac; 4=JJ weaker variant of pLtetO-1 (further referred to as DiffJJ); 5=pLtetO-1

FIG. 10. EMG2 post-transduction lysis kinetics when mixed with P4-like particles. A) at MOI=1; B) at MOI=10. The starting cell density was OD600=0.2. The data in the graph is normalized against the dilution media used for bringing the particles to set MOI: LB+80 mM MgCl2. DiffJJ-Test=Lysis kinetics of EMG2 mixed with P4-like particles encapsulating a lytic cassette regulated by the JJ weaker variant of the pLtetO-1 promoter; DiffJJ/pLtetO1-LacI/LacIq/tac=Lysis kinetics of EMG2 mixed with P4-like particles encapsulating a lytic cassette and the PE antigen regulated by the JJ weaker variant of the pLtetO-1 promoter (DiffJJ) or pLtetO1 and by the LacI, LacIq, tac promoters, respectively. x_{dn}=delay in lysis relative to lytic control, DiffJJ_Test at the MOI equal to “n”.

FIG. 11. Antigen expression post-transduction into EMG2 cells; detected at half-time and full-time lysis into the supernatant, respectively. Lane 1=Color Prestained Protein Standard, Broad Range (11-245 kDa); Lane 2=300 ng PE control; Lane 3-4=transduced with P4-DiffJJ-Test (MOI=2); Lane 5-6=transduced with P4-DiffJJ-LacI (MOI=4); Lane 7-8=transduced with P4-pLtetO1-LacIq (MOI=4); Lane 9-10=transduced with P4-pLtetO1-tac (MOI=2); A) Western blot with mouse anti-PE primary antibody and goat anti-mouse HRP secondary antibody; chemiluminescence detected via ImageQuant. B) ImageJ quantification calculating area under the peak based on western blot band density relative to known amount of 300 ng in Lane 2.

FIG. 12. Evidence of increased cargo capacity for P4 with sid knockout; 1) Thermo Scientific™ GeneRuler 1 kb Plus DNA Ladder; 2) Transduced P4 phasmid with cargo (pP4-c2); able to pack into P4-sized capsids; 3) transduced P4 phasmid with sid knockout and large cargo (pP4Δsid-large); unable to pack into P4 sized capsids.

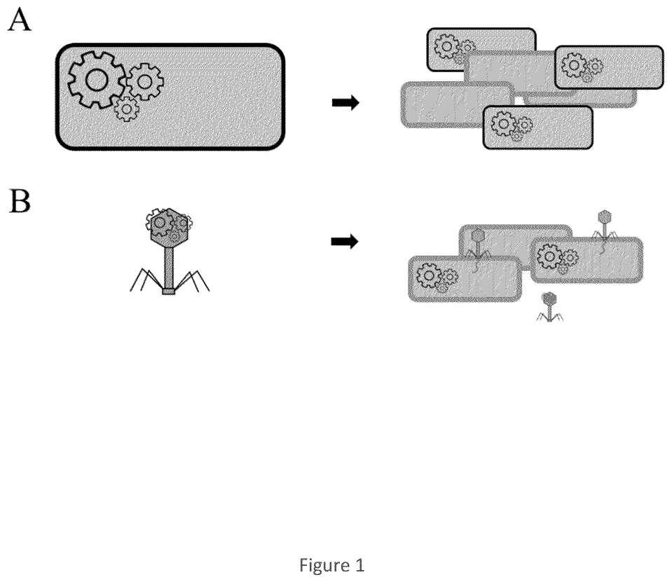

### DETAILED DESCRIPTION

The present invention discloses a engineered bacteriophage comprising a polynucleotide encoding a heterologous protein under the control of a repressible promoter.

In an embodiment the repressible promoter is switched off when a repressor binds to the promoter. Thus the repressible promoter can be switched off during production of bacteriophage, allowing enhanced cargo stability and/or increased production yield. During use of the engineered bacteriophage for example when administered to a subject (e.g. a human subject), the repressible promoter is not bound to a repressor and normal levels of expression of the heterologous protein takes place.

As used herein a “heterologous protein” is a protein which is not expressed in the native bacteriophage.

In an embodiment, the heterologous protein is a therapeutic or prophylactic agent, for example a protein that is capable of alleviating an infection by killing an infectious agent or is capable of generating an immune response against an infectious agent.

In an embodiment, the heterologous protein is an antigen and/or immunomodulatory agent. For example the antigen may be in the form of a fusion protein in which an immunomodulatory agent is fused to the antigen. Alternatively, the bacteriophage comprises a gene encoding an antigen under the control of a repressible promoter and a second gene under the control of a separate repressible promoter. In an embodiment, the heterologous protein is a CRISPR protein or a lysin.

In an embodiment, the expression of the heterologous protein in a bacterial host cell leads to metabolic strain in the bacterial host cell.

In an embodiment the heterologous protein has a molecular weight of at least 10 kDa, 20 kDa, 30 kDa, 40 kDa, 50 kDa, 60 kDa, 70 kDa, 75 kDa or 80 kDa. It has been noticed that the expression of larger proteins can lead to a higher metabolic strain on a bacterium, leading to a reduction in the resources available to make bacteriophage. In an embodiment, the advantages of the present invention are increased where the size of the heterologous protein is larger.

### Bacterial Protein Antigens

In an embodiment, the engineered bacteriophage comprises a polynucleotide that encodes a bacterial antigen, for example a bacterium Gram positive bacterial antigen or Gram negative bacterial antigen or a mycobacterial antigen. In an embodiment, the antigen is naturally present in a bacterium that is capable of being infected by the bacteriophage. In this case, the engineered bacteriophage is particularly suitable for a prime and kill approach in which the engineered bacteriophage comprises a polynucleotide encoding an antigen and a protein capable of killing the bacterium. In this case the bacteriophage is able to express the antigen to raise an immune response against a pathogenic bacterium and kill the pathogenic bacterium through the expression of a killing protein, for example a lysin or CRISPR/Cas system proteins or nucleases.

In an embodiment, the heterologous protein, for example an antigen, is a Staphylococcus aureus, Streptococcus e.g. Streptococcus pneumoniae, Shigella, Pseudomonas e.g. Pseudomonas aeruginosa, Cutibacterium (Propionibacterium), Acinetobacter, Neisseria meningitidis, E. coli, C. difficile, C. acnes (P. acnes), K. pneumoniae, Neisseria gonorrhoea and Fusobacterium, for example Fusobacterium nucleatum antigen, P. gingivalis or Mycobacterium avium paratuberculosis (MAP) antigen.

Examples of C. difficile proteins suitable for the invention include Toxin A (TcdA), Toxin B (TcdB), fusion proteins containing portions of Toxin A and Toxin B (for example as described in WO 12/163817 or WO 12/28741) or fragment of Toxin A or ToxinB (for example, as described in WO 20/237090) binary toxin proteins CdtA and/or CdtB or fusion proteins thereof (for example as described in WO 15/197737).

Examples of staphylococcal proteins suitable for the invention include SitC/MntC/saliva binding protein, EbhA, EbhB, Elastin binding protein (EbpS), EFB (FIB), SBI, ClfA, SdrC, SdrG, SdrH, Lipase GehD, SasA, FnbA, FnbB, Cna, CIfB, FbpA, Npase, IsaA/PisA, SsaA, EPB, SSP-1, SSP-2, HBP, Vitronectin binding protein, fibrinogen binding protein, coagulase, Fig and MAP, IsdA, IsdB, HarA, MntC, alpha toxin (Hla), detoxified alpha toxin point mutation, optionally with a point mutation at H35, RNA III activating protein (RAP), protein A, a variant of protein A. In an embodiment, the heterologous gene encodes a protein described in WO 06/32472.

Examples of pneumococcal proteins suitable for the invention include proteins from the Poly Histidine Triad family (PhtX), Choline Binding Protein family (CbpX), CbpX truncates, LytX family, LytX truncates, CbpX truncate-LytX truncate chimeric proteins (or fusions), pneumolysin (Ply), PspA, PsaA, Sp128, Sp101, Sp130, Sp125 and Sp133. A further embodiment comprises 2 or more proteins selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), pneumolysin (Ply), PspA, PsaA, and Sp128. In one more embodiment, the immunogenic composition comprises 2 or more proteins selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), pneumolysin (Ply), and Sp128, for example PhtD and peumolysin.

The Pht (Poly Histidine Triad) family comprises proteins PhtA, PhtB, PhtD, and PhtE. The family is characterized by a lipidation sequence, two domains separated by a proline-rich region and several histidine triads, possibly involved in metal or nucleoside binding or enzymatic activity, (3-5) coiled-coil regions, a conserved N-terminus and a heterogeneous C terminus. It is present in all strains of pneumococci tested. Homologous proteins have also been found in other Streptococci and Neisseria. In one embodiment of the invention, the Pht protein of the invention is PhtD. It is understood, however, that the terms Pht A, B, D, and E refer to proteins having sequences disclosed in the citations below as well as naturally-occurring (and man-made) variants thereof that have a sequence homology that is at least 90% identical to the referenced proteins. Optionally it is at least 95% identical or at least 97% identical.

Examples of Moraxella catarrhalis protein antigens suitable for the invention are: OMP106 [WO 97/41731 (Antex) & WO 96/34960 (PMC)]; OMP21 or fragments thereof (WO 0018910); LbpA &/or LbpB [WO 98/55606 (PMC)]; TbpA &/or TbpB [WO 97/13785 & WO 97/32980 (PMC)]; CopB [Helminen M E, et al. (1993) Infect. Immun. 61:2003-2010]; UspA1 and/or UspA2 [WO 93/03761 (University of Texas)]; OmpCD; HasR (PCT/EP99/03824); PilQ (PCT/EP99/03823); OMP85 (PCT/EP00/01468); lipo06 (GB 9917977.2); lipo10 (GB 9918208.1); lipo11 (GB 9918302.2); lipo18 (GB 9918038.2); P6 (PCT/EP99/03038); D15 (PCT/EP99/03822); OmpIA1 (PCT/EP99/06781); Hly3 (PCT/EP99/03257); and OmpE. Examples of non-typeable Haemophilus influenzae antigens or fragments thereof which can be included in a combination vaccine (especially for the prevention of otitis media) include: Fimbrin protein [(U.S. Pat. No. 5,766,608-Ohio State Research Foundation)] and fusions comprising peptides therefrom [eg LB1(f) peptide fusions; U.S. Pat. No. 5,843,464 (OSU) or WO 99/64067]; OMP26 [WO 97/01638 (Cortecs)]; P6 [EP 281673 (State University of New York)]; TbpA and/or TbpB; Hia; Hsf; Hin47; Hif; Hmw1; Hmw2; Hmw3; Hmw4; Hap; D15 (WO 94/12641); P2; and P5 (WO 94/26304).

Examples of Fusobacterium protein antigens include FadA for example FadA from Fusobacterium nucleatum. In an embodiment, the FadA is a full length FadA, mature FadA (mFadA) in which the signal peptide is removed or a fragment of FadA. Examples of FadA polypeptides or nucleic acids encoding such a FadA are disclosed in U.S. 63/060,264. In an embodiment, the FadA protein is from a particular subspecies of F. nucleatum, for example from subspecies, nucleatum, animals, vincentii, polymorphan or fusiforme, for example from F. nucleatum nucleatum.

In an embodiment, the heterologous protein has the amino acid sequence disclosed in the appropriate reference. Alternatively, the polypeptide has an amino acid sequence that is at least 80%, 85%, 90%, 91%, 92%, 93%, 94%, 95%, 96%, 97%, 98% or 99% identical to the sequence disclosed in the appropriate reference. In an embodiment, the polypeptide is or comprises a fragment a sequence disclosed in the accompanying reference wherein the fragment comprises at least 30, 40, 50, 60, 70, 80, 90, 100, 120, 150, 170, 200 or 250 contiguous amino acids from the appropriate referenced sequence.

### Repressible Promoters

In an embodiment, the engineered bacteriophage contains a repressible promoter selected from the group consisting of a pLtetO1promoter, pLtetO-1 promoter variants (W, S, E, R, JJ, P and II), tac promoter, pVanCC promoter, PhIF promoter, CymRC promoter, BetI promoter, Tlg promoter and 3B5C promoter. These promoters are taught in Alper et al PNAS (2006) 102, 12678-12683 and Meyer et al Nature Chemical Biology (2019) 15, 196-204. The skilled person is well aware of many repressible promoters which can be used in different bacterial strains. These include many repressible promoters for use in E. coli which are available from https://parts.igem.org. Repressible promoters that can be used in S. aureus include tetracycline and Lac promoters and others (I. R. Monk et al (2012) ASM Journal Vol 3, NO. 2 e00277.11, P. Gao et al Front Microbiol. (2016) Vol 7, Article 1344). Repressible promoters that can be used in P. aeruginosa include Lac promoter (H. D. Kulasekara et al (2004) Molecular Microbiology 55, 368-380). The pTet promoter has been used in both S. pneumoniae and C. difficile (M. Stieger et al Gene 226, 243-251 (1999), K. N. McAllister et al (2017) Sci. Rep. 7:14672).

A repressible promoter as used herein, is a promoter in which drives the expression of a gene when a repressor is bound to the repressible promoter. The repressible promoter can be a strong promoter or a weak promoter in the absence of repressor. In the presence of repressor, transcription of a gene adjacent to the repressible promoter is decreased by at least 10-fold, 50-fold, 100-fold, 500-fold or 1000-fold in comparison to the level of transcription in the absence of the repressor.

In an embodiment, the repressible promoter is capable of being repressed during the production of bacteriophage. The addition of certain compounds such as 2,4-diacetylphophloroglucinol (DAPG), cuminic acid (Cuma), 3-oxohexanoyl-homoserine lactone (OC6), vanillic acid (Van), isopropyl β-d-1-thiogalactopyranoside (IPTG), anhydrotetracycline (aTc), I-arabinose (Ara), choline chloride (Cho), naringenin (Nar), 3,4-dihydroxybenzoic acid (DHBA), sodium salicylate (Sal), and 3-hydroxytetradecanoyl-homoserine lactone (OHC14) to the growth media, can lead to the repressor being detached from the promoter so that transcription from the repressible promoter can take place. During production of the bacteriophage, it is beneficial to use a bacterial production strain which expresses a repressor in the absence of the compounds that could detach the repressor from the repressible promoter. In this way, expression of the heterologous promoter is repressed in the bacterial production strain. However, in an in vivo situation, a repressor will not be expressed in commensal or pathogenic bacteria found in a human/mammalian organism so that the heterologous protein will be expressed. Therefore when the engineered bacteriophage is used in a prime only or prime and kill scenario, the absence of repressors allows good expression of a heterologous protein such as an antigen or a protein capable of killing a bacterium.

In an embodiment, the promoter is capable of being repressed during the production of bacteriophage through the expression of the corresponding repressor gene in the production strain. For example, the pVanCC promoter is repressed by the expression of the VanR repressor gene. Marionette strains allow repression of expression from several repressible promoters (Meyer et al Nature Chemical Biology (2019) 15, 196-204).

The repressible promoter optionally provides the advantage of increased yield of production of the bacteriophage and/or the advantage of increased stability of the cargo carried by the bacteriophage. Therefore, in an embodiment, the engineered bacteriophage of the invention is manufactured at a higher yield than the corresponding engineered bacteriophage comprising a polynucleotide encoding a heterologous protein under the control of a constitutive promoter.

In an embodiment, the engineered bacteriophage is manufactured at a higher yield of PFU than that of a corresponding engineered bacteriophage comprising a polynucleotide encoding a heterologous protein under the control of a constitutive promoter. In an embodiment, the yield of PFU is at least 10-fold, 100-fold, 1,000-fold or 10,000-fold higher than that of a corresponding engineered bacteriophage comprising a polynucleotide encoding a heterologous protein under the control of a constitutive promoter.

In an embodiment, the engineered bacteriophage is more stable than a corresponding engineered bacteriophage in which the heterologous protein is under the control of a constitutive promoter.

As used herein, “stable” and “more stable” means that a bacteriophage cargo (e.g. a heterologous protein) is maintained with a higher degree of correct nucleotide sequence during transduction and/or production of a bacteriophage preparation under conditions where the repressible promoter is repressed, in comparison with a corresponding bacteriophage using a constitutive promoter.

In an embodiment, the engineered bacteriophage is considered more stable when more than 80%, 90%, 95%, 97%, 98% or 99% or 100% of transductants retain the correct sequence of the heterologous protein. The degree of stability can easily be assessed by the skilled person by sequencing the transductants and noting any changes from the expected sequence of the heterologous protein.

### Headsize Regulation

A further aspect of the invention is an adaptation of a bacteriophage, for example a satellite bacteriophage, for example P4, which increases the cargo capacity of the bacteriophage polynucleotide. The inventors have utilised the deletion of at least part of a headsize regulator gene, for example a sid gene, to allow a larger polynucleotide to be inserted into bacteriophage head. This results in the advantage of increasing the size of the heterologous protein that can be encoded by the polynucleotide.

Accordingly there is provided, an engineered bacteriophage (for example a satellite bacteriophage) comprising a polynucleotide comprises a packaging sequence and at least one gene encoding a heterologous protein under the control of a promoter wherein at least part of a headsize regulator gene is deleted.

In an embodiment, the headsize regulator gene is a sid gene. In particular for P4 bacteriophages, the headsize regulator gene is a sid gene.

The increased maximum size of the cargo capacity is beneficial in allowing multiple genes to be present in the polynucleotide, allowing multiple heterologous proteins to be encoded in the polynucleotide. The approach of deleting at least part of a headsize regulator gene is therefore useful where multiple antigens are encoded as heterologous proteins or where a prime and kill approach requires the presence of genes encoding at least one antigen and at least one killing protein. Accordingly, an embodiment provides an engineered bacteriophage wherein the polynucleotide comprises at least 2, 3, 4, 5, 6, 7, 8, 9 or 10 genes encoding a heterologous protein.

In an embodiment, the increased maximum size of the cargo capacity is beneficial in allowing at least one large heterologous protein to be encoded in the polynucleotide. Accordingly, in an embodiment, the engineered bacteriophage of any one of claims 89-91 wherein the at least one gene encoding a heterologous protein is at least 2 kb, 3 kb, 4 kb, 5 kb, 6 kb, 8 kb, 10 kb, 15 kb or 20 kb in size.

### Use of Bacteriophage to Induce an Immune Response in a Prime Only Strategy

The engineered bacteriophages according to the invention are useful for use in a prime only strategy in which a bacteriophage targets a commensal bacterium and inserts nucleic acid to allow the commensal bacteriophage to become an antigen producer, secreting or excreting antigen into the environment so that an immune response is elicited without killing the commensal bacterium.

Accordingly, in an embodiment, the engineered bacteriophage of the invention is capable of binding to and entering a commensal bacterium. The commensal bacterium is optionally selected from the group consisting of E. coli, S. epidermidis, C. acnes, F. nucleatum, and P. gingivalis.

### Use of Bacteriophage to Induce an Immune Response in Pathogenic Bacteria

In a further strategy, the bacteriophage is used to target a pathogenic bacterium, by inserting a polynucleotide into a pathogenic bacterium. In an embodiment, the polynucleotide encodes a protein which is capable of killing the host bacterium, for example bacteriophage lysins or CRISPR/Cas system proteins, either of which is under the control of a repressible promoter. In an embodiment, a prime and kill strategy is used in which the bacteriophage targets a pathogenic bacterium by inserting a polynucleotide encoding a protein capable of killing the host bacterium (killing protein) and a protein antigen into the host bacterium. The host bacterium is able to produce antigen to elicit an immune response against the targeted host bacterium or a further pathogenic bacterium before being killed by expression of the killing protein. In this case, the protein antigen is either released from the host bacterium by excretion or secretion, or is displayed on the surface of the host bacterium or is released upon killing of the host bacterium. In this way the bacteriophage is able to reduce the numbers of pathogenic bacteria present by directly killing the pathogenic bacterium and by eliciting an immune response against a pathogenic bacterium allowing subsequent indirect killing of the pathogenic bacteria.

In an embodiment the engineered bacteriophage is capable of binding to and entering a pathogenic bacterium selected from the group consisting of Staphylococcus aureus, Streptococcus pneumoniae, Shigella flexneri, Shigella sonnei, Pseudomonas aeruginosa, Propionibacterium, Acinetobacter, Neisseria meningitidis, E. coli, P. aeruginosa, C. difficile, P. acnes, K. pneumoniae, N. gonorrhoea. In an embodiment the pathogenic bacterium is a Staphylococcus aureus.

The engineered bacteriophage used in a prime and kill approach typically contains a polynucleotide which comprises a gene encoding a prophylactic agent and a killing gene encoding a protein that is capable of killing a host bacterium. The inventors have further noted that the prime and kill strategy can benefit from the use of promoters of a similar strength driving expression from the gene encoding a prophylactic agent and the killing gene.

In an embodiment, the gene encoding a prophylactic agent and the killing gene are under the control of two promoters having the same strength. In an embodiment, the gene encoding a prophylactic agent and the killing gene are under the control of two promoters having similar strength. In an embodiment, the gene encoding the prophylactic agent and the killing gene are under the control of two strong promoters. In an embodiment, the gene encoding a prophylactic agent and the killing gene are under the control of two weak promoters.

### Bacteriophage

In an embodiment, the engineered bacteriophage is adapted to bind to a bacterium through modification of a gene encoding a bacteriophage tail fibre/plate. This allows entry into any one of the bacteria (commensal or pathogenic) mentioned above.

In an embodiment, the engineered bacteriophage is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae, for example a myoviridae or a siphoviridae.

In an embodiment, the engineered bacteriophage polynucleotide encoding a heterologous antigen is more stable in the engineered bacteriophage than the corresponding polynucleotide encoding the heterologous protein under the control of a constitutive promoter.

In an embodiment which is compatible with the prime and kill strategy, the heterologous protein, after expression, is directed to the surface of a bacterium infected by the bacteriophage, for example through a the heterologous protein comprising an appropriate signal peptide capable of guiding the heterologous to the correct position in the host bacterium.

In an embodiment the heterologous protein, after expression is released into the cytoplasm of a bacterium infected by the bacteriophage. In this case, the heterologous protein is optionally released from the cytoplasm of the bacterium on death of the bacterium.

An embodiment discloses an engineered bacteriophage capable of binding to (for example, adapted to bind to) a bacterium but incapable of producing progeny within the bacterium, wherein the engineered bacteriophage comprises a phage genome polynucleotide including a gene encoding at least one heterologous antigen(s) under the control of a promoter, wherein the gene encoding the at least one heterologous antigen optionally contains a signal sequence capable of driving the release of the heterologous antigen from the bacterium. The phage genome polynucleotide contains a packaging sequence to allow the production of the engineered bacteriophage. The phage genome polynucleotide need contain no further bacteriophage genes. The phage genome polynucleotide optionally contains a phage origin of replication. The engineered bacteriophage typically comprises a viral head made up of capsid protein(s) and comprising a engineered phage genome polynucleotide, tail/plate structures containing means to bind to a bacterial host cell and means to insert the engineered phage genome polynucleotide into a host cell. The genome polynucleotide is engineered to retain at least the packaging signal sequences and optionally sequences essential for genome replication, transcription and translation of the bacteriophage genome. However other parts of the bacteriophage genome may be replaced with one or more genes encoding a heterologous antigen. It is preferred that the bacteriophage genome does not contain genes encoding proteins which allow the lysogenic cycle to proceed. It is also preferred that the bacteriophage genome of the invention does not contain all the genes that allow replication and release of viable bacteriophage. It is also preferred that the bacteriophage genome not encode the lytic machinery of the bacteriophage. Therefore genes that encode proteins involved with the initiation of the lysogenic cycle or that encode some of the structural proteins of the bacteriophage may be deleted and/or may be replaced with genes encoding at least one heterologous antigen. Optionally, the phage genome polynucleotide contains a packaging sequence and no further genetic material originating from a phage. The packaging signal allows an engineered phage to be made by inserting the genome polynucleotide containing the packaging sequence as well as at least one heterologous antigen gene under the control of a promoter, into a phage particle. Optionally, the engineered bacteriophage comprises a receptor for a bacterium. This is typically a protein from the tail of the bacteriophage which binds specifically to a bacterium, allowing the bacteriophage to bind to the bacterium and insert genome polynucleotide into the bacterium.

As used herein “heterologous” antigen or protein, refers to an antigen or protein that is not present in the wild-type bacteriophage. It may be an antigen from a bacterium, for example a pathogenic bacterium or it may be an antigen from a virus or from a fungus. The antigen is a protein, glycoprotein, lipoprotein or even a saccharide synthesized by encoded enzymes.

By “heterologous pathogen” it is meant a pathogen which is not a bacteriophage.

The term “commensal bacterium” refers to a bacterium which is normally present in the subject to be treated and is not associated with a pathogenic infection/disease.

In an embodiment of the invention the engineered bacteriophage is adapted to bind to the commensal bacterium and insert the bacteriophage genome polynucleotide into said host bacterium. For example, the engineered bacteriophage is adapted to bind to a commensal bacterium through modification of a gene encoding a bacteriophage tail fibre/plate. In such an embodiment, the gene encoding the tail fibre/plate is optionally mutated or substituted to increase the binding of the tail fibre/plate to a selected commensal bacterium. In other embodiments, a bacteriophage which naturally has high affinity for the commensal bacterium is selected and further engineered modifications, as set out in this application, are made to adapt the engineered bacteriophage to produce heterologous antigen in a commensal bacterium.

In an embodiment, the commensal bacterium is a part of skin, genital, oral or gut microbiome. For example, the commensal bacterium is Propionibacterium acnes, Staphylococcus epidermidis, Lactobacillus, Streptococcus gordonii or Escherichia coli. The invention includes the targeting of an E. coli bacterium as part of the gut microbiome, S. epidermidis or P. acnes as part of the skin microbiome, Lactobacillus as part of the genital microbiome or Streptococcus gordonii as part of the oral microbiome.

In an embodiment of the invention, the commensal bacterium is a commensal bacterium found in the intestine, on the skin or in the mouth. Examples of suitable commensal bacteria include staphylococcal, streptococcal, E. coli, P. acnes, or Staphylococcus epidermidis bacterium.

In an embodiment, the engineered bacteriophage targets a commensal bacterium in the intestine, for example an E. coli bacterium. In the case of the commensal E. coli bacteria, the caudovirales are able to bind to the commensal E. coli and insert a genome polynuceotide into the E. coli. The Caudovirales is comprised of three phylogenetically-related families that are discriminated by tail morphology: Myoviridae have long contractile tails, Siphoviridae have long non-contractile tails and Podoviridae have short tails, Examples of E. coli bacteriophages are the coliphages λ (Siphoviridae), T4 (Myoviridae) and T7 (Podoviridae).

In an embodiment, the engineered bacteriophage of the invention targets a commensal bacterium on the skin, for example S. epidermidis or P. acnes.

In an embodiment, the engineered bacteriophage contains a gene encoding the heterologous antigen which is under the control of an early promoter or a strong promoter. Examples of a suitable promoter include a promoter controlling the expression of bacteriophage capsid proteins, optionally the promoter controlling the expression to bacteriophage capsid protein from the original bacteriophage. Alternative strong promoters include promoters from the commensal bacterium which drive the expression of high expression level proteins. This can be simply accomplished by replacing the gene encoding capsid proteins with the gene encoding the heterologous antigen in the engineered bacteriophage. Alternatively a capsid promoter or another promoter, such as a strong promoter can be engineered to a position adjacent to the heterologous antigen genes.

In an embodiment, the engineered bacteriophage of the invention is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae. In certain embodiments, myoviridae or a siphoviridae or a podoviridae are selected. In an embodiment the engineered bacteriophage is a engineered lamda coliphage.

In an embodiment, the heterologous antigen gene contains a signal sequence which directs the protein to be released from the bacterium, either by secretion or excretion (Bacterial Secretion Systems—An Overview Erin Green and Joan Mecsas, Microbiol. Spectr. 2016 February; 4 (1): doi: 10.1128/microbiolspec.VMBF-0012-2015). Examples of suitable signal sequences include type I, II, III, IV or V signal sequences of bacteria. Depending on the choice of bacterium and the choice of antigen, the skilled person is able to determine an appropriate signal sequence to use to accomplish efficient release of the heterologous antigen.

Typical examples include co-translational secretion using a DsbA signal sequence, post-translational secretion using a PelB, OmpA or OmpX signal sequence, Tata secretion pathway using a YdcG, SufI or Yack signal sequence.

In an embodiment, the heterologous antigen is a bacterial protein originating from a Gram positive or Gram negative bacterium, for example a protein capable of generating an effective immune response against a pathogenic bacterium.

In an embodiment wherein the engineered bacteriophage targets a commensal or pathogenic bacterium located in the intestine, the heterologous antigen protein is a protein capable of generating an immune response against a pathogenic bacterium of the human intestine. For example, the heterologous antigen is optionally a protein from C. difficile, Helicobacter pylori, Shigella or ETEC E. coli.

Examples of Clostridium difficile proteins which can be encoded by the engineered bacteriophage include protein comprising at least part of the repeat domain of Toxin A and/or Toxin B of C. difficile. In an embodiment, the fragments of ToxinA and/or Toxin B are not toxic. In particular, fusion proteins containing part of the repeat domain of Toxin A and Toxin B are disclosed in WO 00/61762, EP2753352, U.S. Pat. No. 9,409,974, WO 12/163817, WO 12/163811 and fusion proteins disclosed in these publications are suitable for use as heterologous antigens of the invention. Further C. difficile antigens that can be used as heterologous antigen are binary toxin (CdtA and/or CdtB WO 15/197737) other C. difficile proteins disclosed in WO 14/045226 or WO 15/61529.

In other embodiments, the engineered bacteriophage of the invention contains a gene encoding a heterologous antigen protein that is a viral protein or a fungal protein.

In an embodiment, the heterologous antigen is a staphylococcal, streptococcal, Shigella, Pseudomonas, Propionibacterium, Acinetobacter or meningococcal antigen or an antigen from E. coli, H. pylori, P. aeruginosa, C. difficile, P. acnes, K. pneumoniae, N. gonorrhoea.

In an embodiment, the engineered bacteriophage of the invention comprises a phage genome polynucleotide including at least 2, 3, 4, 5, 6, 7, 8, 9 or 10 genes encoding at least 2, 3, 4, 5, 6, 7, 8, 9 or 10 heterologous antigens. These genes are optionally added to the genome polynucleotide at the same position as the deletion of other genes from the genome polynucleotide. Since the capsid proteins are under the control of a strong promoter, one option is to replace the capsid protein gene(s) with one or more heterologous protein antigens, whose expression is driven by the strong promoter. Further or alternatively, genes encoding lytic cycle or lysogeny genes can be replaced by one or more heterologous antigen encoding genes. In an embodiment, the phage genome polynucleotide contains at least one packaging signal and at least, 1, 2, 3, 4, 5, 6, 7, 8, 9 or 10 heterologous antigens under the control of a promoter, for example a strong promoter.

In an embodiment, the engineered bacteriophage does not lyse the commensal bacterium, so that antigen production and release from the commensal bacterium can continue. In such embodiments, the engineered bacteriophage does not contain genes encoding proteins which would either lyse the bacterium or act as a antibacterial toxin, for example non-lytic antimicrobial peptides (AMPs) are not encoded by the genome polynucleotide.

A further aspect of the invention is the genetic material of the engineered bacteriophage. The invention therefore provides, an engineered bacteriophage genome polynucleotide comprising a heterologous antigen gene encoding a heterologous antigen under the control of a promoter wherein the heterologous antigen gene optionally comprises a signal sequence capable of driving the release of the heterologous antigen from a commensal bacterium, wherein at least one gene encoding a capsid protein is deleted, at least one gene encoding a lytic machinery protein is deleted and optionally at least one gene encoding a lysogeny machinery protein is deleted.

The invention also provides, an engineered bacteriophage genome polynucleotide comprising a packaging signal sequence and a heterologous antigen gene encoding a heterologous antigen under the control of a promoter wherein the heterologous antigen gene optionally comprises a signal sequence capable of driving the release of the heterologous antigen from a commensal bacterium, wherein no gene encoding a capsid protein is present, no gene encoding a lytic machinery protein is present and no gene encoding a lysogeny machinery protein is present.

In an embodiment, the invention discloses an engineered bacteriophage genome polynucleotide comprising a packaging signal sequence, a phage origin of replication and a gene encoding a heterologous antigen under the control of a promoter which comprises a signal sequence capable of releasing the heterologous antigen from a bacterium.

In an embodiment, the invention discloses an engineered bacteriophage genome polynucleotide comprising a packaging signal sequence, a phage origin of replication, a bacterial origin of replication and a gene encoding a heterologous antigen under the control of a promoter which comprises a signal sequence capable of releasing the heterologous antigen from a bacterium.

In an embodiment, the invention discloses an engineered bacteriophage genome polynucleotide comprising a packaging signal sequence, a phage origin of replication, a bacterial origin of replication, a selection marker and a gene encoding a heterologous antigen under the control of a promoter which comprises a signal sequence capable of releasing the heterologous antigen from a bacterium.

In an embodiment, the invention discloses an engineered bacteriophage genome polynucleotide comprising a packaging signal sequence, a phage origin of replication, a bacterial origin of replication and a gene encoding a heterologous antigen under the control of a promoter which comprises a signal sequence capable of releasing the heterologous antigen from a bacterium and does not contain a gene encoding a phage capsid protein.

In an embodiment, the invention discloses an engineered bacteriophage genome polynucleotide comprising a packaging signal sequence, a phage origin of replication, a bacterial origin of replication and a gene encoding a heterologous antigen under the control of a promoter which comprises a signal sequence capable of releasing the heterologous antigen from a bacterium and does not contain a gene encoding a phage capsid protein nor a gene encoding a lytic machinery protein.

In an embodiment, the invention discloses an engineered bacteriophage genome polynucleotide comprising a packaging signal sequence, a phage origin of replication, a bacterial origin of replication and a gene encoding a heterologous antigen under the control of a promoter which comprises a signal sequence capable of releasing the heterologous antigen from a commensal bacterium and does not contain a gene encoding a phage capsid protein nor a gene encoding a phage lytic machinery protein nor a gene encoding a phage lysogeny machinery protein.

In an embodiment, the engineered bacteriophage or the engineered bacteriophage genome polynucleotide does not contain a gene encoding a phage capsid protein and/or a phage lytic machinery protein and/or a phage lysogeny machinery protein. For example, the engineered bacteriophage or the engineered bacteriophage genome does not contain a gene encoding a phage capside protein; the engineered bacteriophage or the engineered bacteriophage genome does not contain a gene encoding a phage lytic machinery protein; the engineered bacteriophage or the engineered bacteriophage genome does not contain a gene encoding a phage lysogeny machinery protein; the engineered bacteriophage or the engineered bacteriophage genome does not contain a gene encoding a phage lytic machinery protein or a phage lysogeny machinery protein; the engineered bacteriophage or the engineered bacteriophage genome does not contain a gene encoding a phage capsid protein or a phage lytic machinery protein; the engineered bacteriophage or the engineered bacteriophage genome does not contain a gene encoding a phage capsid protein or a phage lysogeny machinery protein; or the engineered bacteriophage or the engineered bacteriophage genome does not contain any of a gene encoding a phage capsid protein or a phage lytic machinery protein or a phage lysogeny machinery protein.

In an embodiment, an engineered bacteriophage incapable of producing progeny has a genome polynucleotide that does not encode a bacteriophage capsid protein.

In an embodiment, an engineered bacteriophage that is incapable of carrying out a lysogenic cycle has a genome polynucleotide that does not encode a phage lysogeny machinery protein.

In an embodiment, an engineered bacteriophage that is incapable of carrying out a lytic cycle has a genome polynucleotide that does not encode a phage lytic machinery protein.

In an embodiment, an engineered bacteriophage that is adapted to bind to bacterium is adapted through a tail fibre/plate which is modified to bind to a selected bacterium, for example a commensal bacterium or a pathogenic bacterium. Optionally, the tail fibre/plate has a mutated sequence and optionally, the mutated sequence increases binding of the engineered bacteriophage to a selected bacterium.

### Genome Polynucleotides

Further provided is an engineered bacteriophage genome polynucleotide comprising a bacteriophage packaging sequence and a gene encoding a heterologous (or non-bacteriophage) protein under the control of a repressible promoter.

In an embodiment, the genome polynucleotide of the invention encodes at least 1 (for example 1, 2, 3, 4, 5, 6, 7, 8, 9, or 10) heterologous protein(s) and its/their expression is driven by at least one promoter such that the commensal bacterium produces sufficient heterologous antigen protein for an immune response to be elicited against the heterologous antigen. The gene encoding the/each heterologous antigen contains a signal peptide that drives the release of the heterologous antigen from the commensal bacterium, allowing the heterologous antigen to encounter the immune system of the host (for example a human) at a distance from the commensal bacterium so that the commensal bacterium is not targeted by the host immune response. The absence of genes encoding capsid protein, lytic machinery and lysogeny machinery proteins leads to the engineered bacteriophage being unable to carry out the production of progeny, a lytic cycle or a lysogenic cycle within the commensal bacterium. The commensal bacterium therefore continues to release heterologous antigen so that a robust immune response is elicited against the heterologous antigen. In an embodiment, the heterologous antigen elicits an immune response which is effective against a pathogen found in the vicinity of the commensal bacterium.

In an embodiment, the genome polynucleotide retains an origin of replication so that the genome polynucleotide can be replicated within the commensal bacterium. For example, in a high-copy number example 500-700 copies of the genome polynucleotide can be found in a commensal cell, in a medium-copy number genome polynucleotide 20-100 copies of the genome polynucleotide are found in a commensal bacterium whereas for a low-copy number bacterial polynucleotide 5-20 copies are found in a commensal or pathogenic bacterium. An increased number of copies allows higher levels of transcription/translation of a heterologous antigen and the release of higher levels of heterologous antigen from the commensal or pathogenic bacterium.

In an embodiment, the gene encoding the heterologous antigen is under the control of a strong promoter. For example, the heterologous antigen is under the control of the promoter associated with the production of capsid protein. This can be achieved by substituting the capsid protein gene for a heterologous antigen gene. Other strong promoters are readily available to the skilled person, for example promoters driving the production of proteins having a high expression level in the commensal or pathogenic bacterium.

In an embodiment, the engineered bacteriophage genome polynucleotide of the invention is engineered from a bacteriophage genome polynucleotide from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae. For example, from a myoviridae or a siphoviridae or a podoviridae genome polynucleotide.

In an embodiment where E. coli is used as the commensal bacterium, the engineered bacteriophage genome polynucleotide is suitably engineered from a T4, T7 or lamda coliphage genome polynucleotide.

In an embodiment, the genome polynucleotide is engineered to delete all genes encoding a capsid protein. In an embodiment, the engineered bacteriophage genome polynucleotide is engineered to delete all genes encoding lytic machinery proteins. In an embodiment, the engineered bacteriophage genome polynucleotide is engineered to delete all genes encoding lysogeny machinery proteins. When deciding how many genes to delete, it should be borne in mind that the size of the genome polynucleotide should be kept at about the same size before and after engineering. Therefore where more or larger heterologous antigens are to be expressed, more no-essential genes should be deleted from the bacteriophage genome.

In an embodiment, the engineered bacteriophage genome polynucleotide retains the origin of replication so that replication of the bacteriophage genome polynucleotide can occur, leading to potentially higher levels of heterologous antigen expression.

In an embodiment, the origin of replication is deleted from the bacteriophage genome polynucleotide, leading to no replication of the genome polynucleotide. This may be appropriate where lower levels of heterologous antigen are required or where more control of the level of expression of the heterologous antigen is required.

In an embodiment, the engineered bacteriophage genome polynucleotide retains the origin a replication and all genes encoding proteins required to allow replication of the bacteriophage genome polynucleotide within a commensal bacterium.

Any heterologous antigen can be expressed in a commensal bacterium using the present invention. In an embodiment the heterologous antigen is associated with an infectious disease or with cancer. In an embodiment, the heterologous antigen protein is a viral protein or a fungal protein or a bacterial protein originating from a Gram positive or Gram negative bacterium. Where more than 1 (for example 2, 3, 4, 5, 6, 7, 8, 9 or 10) heterologous antigens are encoded, it is possible to express both viral and bacterial, or viral and fungal or bacterial and fungal or bacterial, viral and fungal heterologous antigen from the same commensal bacterium. This is particularly suitable to tackle infections like otitis media which contain both bacterial and viral elements.

In an embodiment, the heterologous antigen protein is a protein capable of generating an immune response against a pathogenic bacterium in the human gut. For example a Clostridium difficile, H. pylori or Shigella infection.

In an embodiment, the heterologous antigen is capable of generating an immune response against C. difficile infection. For example, the genome polynucleotide suitably comprises a gene encoding a protein capable of generating an immune response against C. difficile, for example C. difficile toxin A, C. dificile Toxin B, fragments of C. difficile Toxin A and/or C. difficile Toxin B or fusion proteins comprising fragments of C. difficile Toxin A and C. difficile Toxin B. In an embodiment, the genome polynucleotide comprises genes encoding one or more of the proteins disclosed in WO 12/163817, WO 12/163811, WO 14/96393, WO 15/197737 or WO 14/45226.

In an embodiment, the heterologous antigen is capable of generating an immune response against Shigella, Salmonella, H. pylori or pathogenic E. coli. In this case, the commensal bacterium is suitably a E. coli from the human gut.

In an embodiment, the heterologous antigen is a staphylococcal, streptococcal, Shigella, Pseudomonas, Propionibacterium. acnes, Acinetobacter or meningococcal protein or a protein from E. Coli, P. aeruginosa, C. difficile, K. pneumoniae, or N. gonorrhoea antigen.

In an embodiment, the heterologous antigen gene comprises a signal sequence which is capable of driving release of the heterologous antigen from the commensal bacterium by secretion or by excretion. The release of the heterologous antigen from the commensal bacterium after expression ensures that an immune response may be elicited against the heterologous antigen without the immune response being directed at the commensal bacterium. For example, a class I, II, III, IV or V signal sequence is attached to the heterologous antigen. The skilled person is aware of many signal sequences which would be suitable for use in a particular commensal bacterium or for a particular heterologous antigen.

In an embodiment, the engineered bacteriophage genome polynucleotide of the invention comprises a gene encoding an adjuvant. In an embodiment, the adjuvant is a TLR5 agonist; for example flagellin or a fragment thereof comprising at least 7, 10, 20, 30, 40, 50, 70, 100 or 200 continuous amino acids. In an embodiment, the more immunogenic epitopes (in the hypervariable region) of flagellin are removed to result in a fragment of flagellin which retains adjuvant activity but is less immunogenic (Nempont C et al J. Imunol. (2008) 181 (3) 2036-2043). Flagellin's hypervariable central region is not required for TLR5 agonist activity so deletion of this region allows adjuvant activity to be retained. For example for FliC (accession number AAL20871) the deletion of amino acids 204-292, 191-352 or 174-400 did not reduce TLR5 activity (Nempont C et al J. Imunol. (2008) 181 (3) 2036-2043).

In an embodiment, expression of the adjuvant is under the control of a strong promoter, for example the capsid protein promoter in the bacteriophage. In an embodiment, the adjuvant is expressed as a fusion protein with the heterologous antigen. For example, the gene encoding the heterologous antigen is fused to a gene encoding the adjuvant, optionally flagellin (or fragment thereof) so that a single fusion protein is released from the commensal bacterium due to the signal peptide which forms part of the fusion protein gene.

In an embodiment, the engineered bacteriophage genome polynucleotide comprises a gene encoding a receptor for a commensal bacterium, which is optionally a tail fibre or a plate. Alternatively, the receptor for a commensal bacterium is not encoded by the engineered bacteriophage genome polynucleotide, in which case, the receptor for a commensal polynucleotide is encoded in a packaging cell line which is used in the production of the engineered bacteriophage.

In an embodiment, the heterologous antigen protein is not naturally expressed in a commensal bacterium that is infectable by the engineered bacteriophage.

In a preferred embodiment, engineered bacteriophage genome polynucleotide comprises a packaging signal.

In an embodiment, the engineered bacteriophage comprises a capsid, a genome polynucleotide, and a means of inserting its genome polynucleotide into a commensal bacterium wherein the genome polynucleotide contains at least one gene encoding at least one heterologous antigen under the control of a promoter, a packaging signal and a phage origin of replication.

In an embodiment, the engineered bacteriophage comprises a capsid, a genome polynucleotide, and a means of inserting its genome polynucleotide into a commensal bacterium wherein the genome polynucleotide contains at least one gene encoding at least one heterologous antigen under the control of a promoter, a packaging signal, a phage origin of replication and a bacterial origin of replication.

In an embodiment, the engineered bacteriophage comprises a capsid, a genome polynucleotide, and a means of inserting its genome polynucleotide into a commensal bacterium wherein the genome polynucleotide contains at least one gene encoding at least one heterologous antigen under the control of a promoter, a packaging signal, a phage origin of replication, a bacterial origin of replication and a selection marker.

### Bacteriophage Composition

In an embodiment, the bacteriophage is an engineered bacteriophage, for example, a bacteriophage where parts of the bacteriophage genome have been deleted. In an embodiment, deleted bacteriophage genetic material is replaced with further genes encoding at least one heterologous protein (for example a protein allowing binding to a mammalian receptor or an enzyme capable of degrading biofilm).

In an embodiment, the engineered bacteriophage of the invention is incapable of carrying out a lysogenic cycle or a lytic cycle or both a lysogenic and a lytic cycle. This is optionally achieved by the deletion of genes encoding bacteriophage capsid proteins being absent from the bacteriophage genome and/or genes encoding proteins essential for the lysogenic cycle being absent from the bacteriophage genome. In an embodiment, the engineered bacteriophage is adapted to degrade biofilm.

As used herein “carrying out a lytic cycle” means that the bacteriophage is not only capable of lysing a host cell but also produces progeny bacteriophage which are released as part of a lytic cycle. Therefore a recombinant bacteriophage is incapable of carrying out a lytic cycle if it cannot produce bacteriophage progeny, even if it expresses a bacteriophage lysin or other means of lysing a host cell.

In an further aspect, the invention discloses a recombinant bacteriophage genome polynucleotide comprising a heterologous gene, for example a killing gene encoding a protein that is capable of killing a host bacterium, for example a bacteriophage lysin or a nuclease associated with CRISPR, optionally Cas9/CRISPR.

In an embodiment, the bacteriophage genome comprises at least one bacteriophage packaging sequence, for example a cos sequence. In an embodiment, the bacteriophage genome comprises a bacteriophage or bacterial origin of replication.

In an embodiment, the bacteriophage genome contains a killing gene under the control of a promoter, optionally a weak or late promoter.

In an embodiment, the engineered bacteriophage is from a bacteriophage family selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae, for example from a myoviridae or a siphoviridae.

In an embodiment, the engineered bacteriophage genome is missing at least one gene associated with a lysogenic cycle which is for example inactivated or deleted. In an embodiment at least one gene encoding a bacteriophage structural protein, for example a capsid protein gene, is deleted. In an embodiment, the recombinant bacteriophage genomic polynucleotide comprises a gene encoding a protein capable of degrading biofilm.

### Engineered Bacteriophage Production Processes

In an embodiment, there is provided a process for producing an engineered bacteriophage preparation containing a step of producing the engineered bacteriophage of the invention under conditions where expression of the heterologous protein is repressed. The presence of a repressible promoter driving the expression of a heterologous protein has been shown by the inventors to result in greater stability of the genetic cargo (e.g. a nucleic acid encoding a heterologous protein) and optionally an increased yield of bacteriophage. The repression is optionally achieved by using a production strain of bacteria which expresses a repressor which is capable of binding to the repressible promoter.

In an embodiment, there is provided a process for producing a engineered bacteriophage comprising a polynucleotide containing a gene encoding a heterologous (or non-bacteriophage) protein under the control of a repressible promoter comprising the steps of i) amplifying the engineered bacteriophage in a bacterial production strain that expresses a repressor of the repressible promoter and ii) isolating the engineered bacteriophage. In an embodiment, the isolated engineered bacteriophage is as described above.

In an embodiment, the process provides an increased yield of engineered bacteriophage, for example wherein the yield of engineered bacteriophage is at least 2, 3, 4, 5, 6, 7, 8, 9, 10, 100 or 1,000 times higher than in a similar production process where a gene encoding a heterologous (or non-bacteriophage) protein is under the control of a constitutive promoter.

In an embodiment, the gene encoding the heterologous (or non-bacteriophage) protein is retained more stably than in a similar production process where a gene encoding a heterologous (or non-bacteriophage) protein is under the control of a constitutive promoter. For example, at least 80%, 90%, 95%, 97%, 98%, 99% or 100% of isolated engineered bacteriophage retain the correct sequence of the gene encoding the heterologous (or non-bacteriophage) protein.

### Pharmaceutical Compositions

A further aspect of the invention is a pharmaceutical composition comprising the engineered bacteriophage or the engineered bacteriophage genome polynucleotide of the invention. The pharmaceutical composition is optionally formulated as a topical treatment, or as a capsule or microcapsule for oral administration, wherein the engineered bacteriophage or the engineered bacteriophage genome polynucleotide is released in the intestine or is formulated as a mouth wash. A capsule or microcapsule formulation optionally contains chitosan-alginate, polymethacrylate copolymer or fatty acid.

A further embodiment of the invention is a pharmaceutical composition comprising the engineered bacteriophage or the engineered bacteriophage genome polynucleotide described above. In an embodiment, the pharmaceutical composition further comprises a pharmaceutically acceptable excipient, for example excipients to allow administration as a topical cream or ointment.

A further aspect of the invention is a vaccine comprising the engineered bacteriophage or the engineered bacteriophage genome polynucleotide of the invention.

The bacteriophage of the invention may be delivered as a pharmaceutical preparation, formulated for effective delivery to the required microbiome.

Where the engineered bacteriophage is required to reach the gut microbiome, microencapsulation is optionally used (WO 06/47871, CA2463827, Yongsheng Ma et al Applied and Environmental Microbiology 74; 4799-4805 (2008)). Microencapsulation allows the engineered bacteriophage to be delivered orally and to be protected from the low pH associated with the stomach. Release occurs in the intestinal fluid where a pH of 6-7, for example 6.8 is achieved. In an embodiment, microencapsulation is microcapsules comprising chitosan-alginate, alginate, polymethacrylate copolymer, alginate-polymethacrylate or using skin milk and fatty acid. Suitable copolymers include anionic copolymers based on methacrylic acid and methyl methacrylate, for example the commercial product Eudragit S100. A lipoidal delivery system is optionally used, in which the engineered bacteriophage are entrapped in cationic lipic vesicles or liposomes.

Where the engineered bacteriophage is to be applied to the skin, the engineered bacteriophage is optionally formulated as a composition for topical treatment. In an embodiment, the engineered phage are combined with a pharmaceutically acceptable carrier, such as an excipient or stabiliser. Examples of pharmaceutically acceptable carrier, excipients and stabilisers include but are not limited to, buffers such as phosphate, citrate and other organic acids; antioxidants including ascorbic acid; low molecular weight polypeptides; protein such as serum albumin and gelatin; hydrophilic polymers such as polyvinylpyrrolidone; amino acids such as glycine, glutamine, asparagine, arginine or lysine; monosaccharides, disaccharides and other carbohydrates including glucose, mannose or dextrins; sugar alcohols such as mannitol or sorbitol; salt-forming counterions such as sodium.

The engineered bacteriophage may be formulated in a variety of product forms, for example a lotion, cream, spray, aerosol, aqueous buffer solution, gel, mask, foam and the like, for topical administration. In one embodiment, the phage are formulated as an aqueous solution of gel. The composition may include water, esters, isopropyl myristate amd isopropyl palmitate; ethers such as dicapryl ether and dimethyl isosorbide; alcohols such as ethanol and isopropanol; fatty acids such as cetyl alcohol, cetearyl alcohol, stearyl alcohol and biphenyl alcohol; isoparaffins such as isooctane, isododecane and isohexadecane; silicone oils such as cyclomethicone, dimethicone, dimethicone cross-polymer, polysiloxanes and their derivatives e.g. organomodified derivatives; polyols such as propylene glycol, glycerine, butyene glycol, pentylene glycol, hexylene glycol; or any combinations of mixtures thereof. Aqueous vehicles may include one or more solvents miscible with water including lower alcohols, such as ethanol, isopropanol, and the like.

In an embodiment, the engineered bacteriophage composition is applied to a biocompatible substrate, for example a substrate comprising a natural fibre such as cotton, eucalyptus, bamboo filter or biocellulose or a combination thereof.

In an embodiment where, the heterologous protein is an antigen, it may be adjuvanted by the presence of a TLR5 agonist. The polynucleotide encodes a TLR5 agonist as well as an antigen. The TLR5 agonist is optionally encoded as part of a fusion protein, for example a fusion protein of an antigen and a TLR5 agonist. In an embodiment, the TLR5 agonist is flagellin. In an embodiment, the heterologous protein is a fusion protein comprising an antigen and flagellin.

### Medical Uses and Methods of Treatment

The invention also discloses a pharmaceutical for use in therapy as well as a vaccine comprising the engineered bacteriophage of the invention or the engineered bacteriophage genome polynucleotide of the invention.

In an embodiment there is provided an engineered bacteriophage according to the invention or a engineered bacteriophage genomic polynucleotide according to the invention, for use in the prophylactic prevention of disease, optionally infectious disease or cancer, for example, a bacterial infection described above. In an embodiment the use comprises the release of a heterologous antigen from a bacterium followed by the priming of an immune response against the heterologous antigen. In an embodiment, the bacterium is a commensal bacterium. In an embodiment, the bacterium is a pathogenic bacterium. In an embodiment, the disease comprises a bacterial infection, for example a S. aureus or C. difficile infection.

Also provided is a method of treatment of a disease comprising the steps of: a) administering the engineered bacteriophage according to the invention or the engineered bacteriophage genomic polynucleotide according to the invention, to a patient in need thereof such that the engineered bacteriophage or engineered bacteriophage genomic polynucleotide contacts a bacterium; b) entry of the genome polynucleotide into the bacterium, and c) expression and release of the heterologous antigen at a sufficient level for an immune response to be elicited against the heterologous antigen. In an embodiment the bacterium is a commensal bacterium. In an embodiment, the bacterium is a pathogenic bacterium. In an embodiment, the disease comprises a bacterial infection, for example a S. aureus infection or a C. difficile infection.

The invention is further disclosed in the paragraphs below:
1. An engineered bacteriophage comprising a polynucleotide encoding a heterologous protein under the control of a repressible promoter.
2. The engineered bacteriophage of paragraph 1 wherein the heterologous protein is a therapeutic or prophylactic agent.
3. The engineered bacteriophage of paragraph 1 or 2 wherein the heterologous protein is an antigen and/or immunomodulatory agent.
4. The engineered bacteriophage of any one of paragraphs 1-3 wherein the heterologous protein is capable of killing a bacterium, for example a bacteriophage lysin or a CRISPR nuclease.
5. The engineered bacteriophage of paragraph 3 wherein the antigen is a protein from a bacterium, for example a Gram positive or Gram negative bacterial polypeptide antigen or a Mycobacterium antigen.
6. The engineered bacteriophage of paragraph 5, wherein the antigen is naturally present in a bacterium that is capable of being infected by the bacteriophage.
7. The engineered bacteriophage of paragraph 5 or 6 wherein the antigen is a Staphylococcus aureus, Streptococcus e.g. Streptococcus pneumoniae, Shigella, Pseudomonas e.g. Pseudomonas aeruginosa, Cutibacterium (Propionibacterium), Acinetobacter, Neisseria meningitidis, E. coli, C. difficile, C. acnes (P. acnes), K. pneumoniae, Neisseria gonorrhoea, Fusobacterium, for example Fusobacterium nucleatum antigen, P. gingivalis or MAP Mycobacterium avium paratuberculosis (MAP) antigen.
8. The engineered bacteriophage of any one of paragraphs 1-7 wherein expression of the antigen in a bacterial host cell leads to metabolic strain in a bacterial host cell.
9. The engineered bacteriophage of any one of paragraphs 1-8 wherein the heterologous protein has a molecular weight of at least 10 kDa, 20 kDa, 30 kDa, 40 kDa, 50 kDa, 60 kDa, 70 kDa, 75 kDa or 80 kDa.
10. The engineered bacteriophage of any one of paragraph 1-9 wherein the promoter is a repressible promoter selected from the group consisting of pLtetO1promoter, pLtetO-1 promoter variants (W, S, E, R, JJ, P and II), tac promoter, pVanCC promoter, PhIF promoter, CymRC promoter, BetI promoter, TIg promoter and 3B5C promoter.
11. The engineered bacteriophage of any one of paragraph 1-10 wherein the engineered bacteriophage is capable of binding to and entering a commensal bacterium.
12. The engineered bacteriophage of paragraph 11 wherein the commensal bacterium is selected from the group consisting of E. coli, S. epidermidis, C. acnes, F. nucleatum, and P. gingivalis.
13. The engineered bacteriophage of any one of paragraphs 1-12 which is capable of binding to and entering a pathogenic bacterium selected from the group consisting of Staphylococcus aureus, Streptococcus pneumoniae, Shigella flexneri, Shigella sonnei, Pseudomonas aeruginosa, Propionibacterium, Acinetobacter, Neisseria meningitidis, E. coli, P. aeruginosa, C. difficile, P. acnes, K. pneumoniae, N. gonorrhoea.
14. The engineered bacteriophage of paragraph 13 wherein the pathogenic bacterium is a Staphylococcus aureus.
15. The engineered bacteriophage of any one of paragraphs 1-14 wherein the engineered bacteriophage is adapted to bind to a bacterium through modification of a gene encoding a bacteriophage tail fibre/plate.
16. The engineered bacteriophage of any one of paragraphs 1-15 wherein bacteriophage is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae.
17. The engineered bacteriophage of paragraph 16, wherein the bacteriophage is a myoviridae or a siphoviridae.
18. The engineered bacteriophage of any one of paragraphs 1-17 wherein the polynucleotide encoding a heterologous antigen is more stable in the engineered bacteriophage than the corresponding polynucleotide encoding a heterologous protein under the control of a constitutive promoter.
19. The engineered bacteriophage of any one of paragraphs 1-18 wherein more than 80%, 90%, 95, 97%, 98% or 99% or 100% of transductants retain the correct nucleotide sequence encoding the heterologous protein.
20. The engineered bacteriophage of any one of paragraphs 1-19 which is manufactured at a higher yield of PFU than that of a corresponding engineered bacteriophage comprising a polynucleotide encoding a heterologous protein under the control of a constitutive promoter.
21. The engineered bacteriophage of paragraph 20 wherein the yield of PFU is at least 10-fold, 100-fold, 1,000-fold or 10,000-fold higher than that of a corresponding engineered bacteriophage comprising a polynucleotide encoding a heterologous protein under the control of a constitutive promoter.
22. The engineered bacteriophage of any one of paragraph 1-21 wherein the polynucleotide comprises a gene encoding a prophylactic agent and a killing gene encoding a protein that is capable of killing a host bacterium.
23. The engineered bacteriophage of paragraph 22 wherein the gene encoding a prophylactic agent and the killing gene are under the control of two promoters having the same strength.
24. The engineered bacteriophage of any one of paragraphs 1-23 wherein the gene encoding the prophylactic agent and the killing gene are under the control of two strong promoters.
25. The engineered bacteriophage of any one of paragraphs 1-23 wherein the gene encoding a prophylactic agent and the killing gene are under the control of two weak promoters.
26. The engineered bacteriophage of any one of paragraphs 1-25 wherein the heterologous protein, after expression, is directed to the surface of a bacterium infected by the bacteriophage.
27. The engineered bacteriophage of any one of paragraphs 1-26 wherein the heterologous protein, after expression, is released from the surface of a bacterium infected by the bacteriophage.
28. The engineered bacteriophage of any one of paragraph 1-27 wherein the heterologous protein, after expression is released into the cytoplasm of a bacterium infected by the bacteriophage.
29. The engineered bacteriophage of paragraph 28 wherein the heterologous protein is released from the cytoplasm of the bacterium on death of the bacterium.
30. The engineered bacteriophage of any one of paragraphs 1-29 wherein the heterologous protein(s) is not expressed as part of a phage coat/capsid protein and optionally, the engineered bacteriophage comprises a receptor for a host bacterium.
31. The engineered bacteriophage of any one of paragraphs 1-30 comprising a polynucleotide including at least 2, 3, 4, 5, 6, 7, 8, 9 or 10 genes encoding at least 2, 3, 4, 5, 6, 7, 8, 9 or 10 heterologous proteins.
32. The engineered bacteriophage of any one of paragraphs 1-31, wherein the engineered bacteriophage is incapable of carrying out a lysogenic cycle and/or a lytic cycle.
33. The engineered bacteriophage of any one of paragraph 1-32, wherein the engineered bacteriophage is adapted to degrade biofilm.
34. The engineered bacteriophage of any one of paragraphs 1-33 wherein the heterologous gene encodes a staphylococcal protein selected from the group consisting of SitC/MntC/saliva binding protein, EbhA, EbhB, Elastin binding protein (EbpS), EFB (FIB), SBI, ClfA, SdrC, SdrG, SdrH, Lipase GehD, SasA, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP, IsdA, IsdB, HarA, MntC, alpha toxin (Hla), detoxified alpha toxin point mutation, optionally with a point mutation at H35, RNA III activating protein (RAP), protein A, a variant of protein A.
35. A process for producing a bacteriophage preparation containing a step of producing the engineered bacteriophage of any one of paragraphs 1-34 under conditions where expression of the heterologous protein is repressed.
36. A process for producing a engineered bacteriophage comprising a polynucleotide containing a gene encoding a heterologous protein under the control of a repressible promoter comprising the steps of i) amplifying the engineered bacteriophage in bacteria in the presence of a repressor of the repressible promoter and ii) isolating the engineered bacteriophage.
37. The process of paragraph 36 wherein the engineered bacteriophage is the engineered bacteriophage of any one of paragraphs 1-34.
38. The process of paragraph 37 wherein the yield of engineered bacteriophage is at least 2, 3, 4, 5, 6, 7, 8, 9, 10, 100 or 1,000 times higher than in the absence of the repressor.
39. The process of paragraph 37 or 38 wherein the gene encoding the heterologous protein is retained more stably than in the absence of the repressor.
40. The process of paragraph 39 wherein at least 80%, 90%, 95%, 97%, 98% or 99% or 100% of transductants retain the correct sequence of the gene encoding the heterologous protein.
41. An engineered bacteriophage genome polynucleotide comprising a bacteriophage packaging sequence and a gene encoding a heterologous protein under the control of a repressible promoter.
42. The engineered bacteriophage genome polynucleotide of paragraph 41 wherein at least one gene encoding a capsid protein is deleted, at least one gene encoding a lytic machinery protein is deleted and optionally at least one gene encoding a lysogeny machinery protein is deleted.
43. The engineered bacteriophage genome polynucleotide of paragraph 41 or 42 wherein the gene encoding the heterologous protein is under the control of a strong promoter.
44. The engineered bacteriophage genome polynucleotide of any one of paragraphs 41-43 which is engineered from bacteriophage genome polynucleotide from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae.
45. The engineered bacteriophage genome polynucleotide of paragraph 44, engineered from a myoviridae or a siphoviridae or a podoviridae genome polynucleotide.
46. The engineered bacteriophage genome polynucleotide of paragraph 45, engineered from a T4, T7 or lamda coliphage genome polynucleotide.
47. The engineered bacteriophage genome polynucleotide of any one of paragraphs 41-46 engineered to delete all genes encoding a capsid protein.
48. The engineered bacteriophage genome polynucleotide of any one of paragraphs 41-47 engineered to delete all genes encoding lytic machinery proteins.
49. The engineered bacteriophage genome polynucleotide of any one of paragraphs 41-48 engineered to delete all genes encoding lysogeny machinery proteins.
50. The engineered bacteriophage genome polynucleotide of any one of paragraphs 41-49 which retains the origin of replication.
51. The engineered bacteriophage genome polynucleotide of any one of paragraphs 41-50 wherein the genome polynucleotide retains a phage origin of replication.
52. The engineered bacteriophage genome polynucleotide of any one of paragraphs 41-51 wherein the genome polynucleotide contains a bacterial origin of replication.
53. The engineered bacteriophage genome polynucleotide of any one of paragraphs 41-52 wherein the genome polynucleotide contains a selection marker.
54. The engineered bacteriophage genome polynucleotide of paragraph 41-53 wherein the bacteriophage genome polynucleotide retains the origin of replication and all genes encoding proteins required to allow replication of the bacteriophage genome polynucleotide within a bacterium.
55. The engineered bacteriophage genome polynucleotide of any one of paragraphs 41-54 wherein the heterologous protein is a bacterial protein originating from a Gram positive or Gram negative bacterium.
56. The engineered bacteriophage genome polynucleotide of paragraphs 41-55 wherein the heterologous protein is a protein capable of generating an immune response against a pathogenic bacterium in the human gut.
57. The engineered bacteriophage genome polynucleotide of paragraph 55 or 56, wherein the heterologous protein is capable of generating an immune response against C. difficile, optionally C. difficile Toxin A or C. difficile Toxin B.
58. The engineered bacteriophage genome polynucleotide of paragraph 55 or 56 wherein the heterologous protein is a protein from C. difficile, H. pylori, Shigella, Salmonella or pathogenic E. coli.
59. The engineered bacteriophage genome polynucleotide of any one of paragraphs 55-58 wherein the heterologous protein contains sequence from C. difficile Toxin A or Toxin B, optionally a fusion protein or C. difficile Toxin A and Toxin B.
60. The engineered bacteriophage genome polynucleotide of paragraph 55 wherein the heterologous protein is a staphylococcal, streptococcal, Shigella, Pseudomonas, Propionibacterium. acnes, Acinetobacter or meningococcal protein or a protein from E. Coli, P. aeruginosa, C. difficile, H. pylori, K. pneumoniae, or N. gonorrhoea antigen.
61. The engineered bacteriophage genome polynucleotide of any one of paragraphs 41-60 including at least 2, 3, 4, 5, 6, 7, 8, 9 or 10 genes encoding at least 2, 3, 4, 5, 6, 7, 8, 9 or 10 heterologous antigens.
62. The engineered bacteriophage genome polynucleotide of paragraph 61 including at least 3 genes encoding at least 3 heterologous antigens.
63. The engineered bacteriophage genome polynucleotide of any one of paragraphs 41-62 wherein the heterologous antigen gene comprises a signal sequence capable of driving the release of the heterologous antigen from a commensal bacterium.
64. The engineered bacteriophage genome polynucleotide of paragraph 63 wherein the signal sequence is capable of driving release of the heterologous antigen from the commensal bacterium by secretion.
65. The engineered bacteriophage genome polynucleotide of paragraph 63 wherein the signal sequence of capable of driving release of the heterologous antigen from the commensal bacterium by excretion.
66. The engineered bacteriophage genome polynucleotide of any one of paragraphs 63-65 wherein the signal sequence is selected from the list consisting of type I, II, III, IV or V signal sequences.
67. The engineered bacteriophage genome polynucleotide of any one of paragraphs 41-66 comprising a gene encoding an adjuvant under the control of a promoter.
68. The engineered bacteriophage of paragraph 67 wherein the adjuvant is a TLR5 agonist 69. The engineered bacteriophage genome polynucleotide of paragraph 67 or 68 wherein the adjuvant is flagellin or a variant thereof which contains a deletion of the hypervariable region.
70. The engineered bacteriophage genome polynucleotide of any one of paragraphs 67-69 wherein expression of the adjuvant is under the control of a strong promoter.
71. The engineered bacteriophage genome polynucleotide of any one of paragraphs 67-70 wherein the adjuvant is expressed as a fusion protein with the heterologous antigen.
72. The engineered bacteriophage genome polynucleotide of any one of paragraphs 41-71 comprising a gene encoding a receptor for a commensal bacterium, optionally a gene encoding a tail fibre/plate.
73. The engineered bacteriophage genome polynucleotide of any one of paragraphs 41-72 wherein the heterologous protein is not naturally expressed in a bacterium that is infectable by the engineered bacteriophage.
74. A use of the engineered bacteriophage of any one of paragraphs 1-34 or the engineered bacteriophage genome polynucleotide of any one of paragraphs 41-73 in the expression and release of the heterologous protein from a bacterium, optionally a commensal bacterium.
75. A pharmaceutical composition comprising the engineered bacteriophage any one of paragraphs 1-34 or the engineered bacteriophage genome polynucleotide of any one of paragraphs 41-73.
76. The pharmaceutical composition of paragraph 75, formulated as a topical treatment.
77. The pharmaceutical composition of paragraph 75, formulated as a capsule for oral administration, wherein the engineered bacteriophage or the engineered bacteriophage genome polynucleotide is released in the intestine.
78. The pharmaceutical composition of paragraph 77 wherein the capsule is a microcapsule.
79. The pharmaceutical composition of paragraph 77 or 78 wherein the capsule contains alginate, chitosan-alginate, polymethacrylate copolymer, alginate-polymethacrylate or fatty acid.
80. The pharmaceutical composition of paragraph 75 formulated as a mouth wash.
81. A vaccine comprising the engineered bacteriophage of any one of paragraphs 1-34 or the engineered bacteriophage genome polynucleotide of any one of paragraphs 41-73.
82. An engineered bacteriophage according to any one of paragraphs 1-34 or a engineered bacteriophage genomic polynucleotide according to any one of paragraphs 41-73, for use in the prevention of disease, optionally infectious disease or cancer.
83. The engineered bacteriophage or engineered bacteriophage genomic polynucleotide for use of paragraph 82, wherein the use comprises the release of a heterologous antigen from a commensal bacterium followed by the priming of an immune response against the heterologous antigen.
84. The engineered bacteriophage or engineered bacteriophage genomic polynucleotide for use of paragraph 82 or 83, wherein the disease comprises a bacterial infection.
85. The engineered bacteriophage or engineered bacteriophage genomic polynucleotide for use of paragraph wherein 84 the disease comprises a bacterial infection, for example a C. difficile infection.
86. A method of treatment of a disease comprising the steps of: a) administering the engineered bacteriophage according to any one of paragraphs 1-34 or the engineered bacteriophage genomic polynucleotide according to any one of paragraphs 41-73, to a patient in need thereof such that the engineered bacteriophage or engineered bacteriophage genomic polynucleotide contacts a commensal bacterium; b) entry of the genome polynucleotide into the commensal bacterium, and c) expression and release of the heterologous antigen at a sufficient level for an immune response to be elicited against the heterologous antigen.
87. The method of treatment of paragraph 86 wherein the disease is a bacterial infection.
88. The method of treatment of paragraph 87, wherein the disease is a C. difficile infection.
89. An engineered bacteriophage comprising a polynucleotide comprises a packaging sequence and at least one gene encoding a heterologous protein under the control of a promoter wherein a headsize regulator gene is inactivated.
90. The engineered bacteriophage of paragraph 89 wherein the headsize regulator gene is a sid gene.
91. The engineered bacteriophage of paragraph 89 or 90 wherein the polynucleotide comprises at least 2, 3, 4, 5, 6, 7, 8, 9 or 10 genes encoding a heterologous protein.
92. The engineered bacteriophage of any one of paragraphs 89-91 wherein the at least one gene encoding a heterologous protein is at least 2 kb, 3 kb, 4 kb, 5 kb, 6 kb, 8 kb, 10 kb, 15 kb or 20 kb in size.
93. The engineered bacteriophage of any one of paragraphs 89-92 which is a satellite bacteriophage, for example P4.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1 Production of a Bacteriophage

### Strains and Plasmids

Escherichia coli C-5545Δcos-Marionette was produced as detailed in Tridgett et al. 2021 [26]. The Escherichia coli EMG2 ATCC 23716 was purchased from the American Type Culture Collection. The backbone vector for all cloning experiments pP4-c2, is described in Ababi et al. 2022. pP4-LacIq-p72 was cloned by two consecutive Gibson Assembly steps. First, a fragment between positions 902 to 2871 was deleted to make space for the insertion 10 of new cargos, then the synthetic p72 gene was introduced between position 349 to 571 on the new construct (Table 1). The remaining plasmids (pP4-pLtetO1-p72, pP4-LacIq-p65, pP4-pLtetO1-p65, pP4-LacIq-Large, pP4-pLtetO1-Large) (Table 1).

TABLE 1
P4 phasmids with cargo sequences.

| Label ID | Sequence 5′-3′ | Backbone |
|---|---|---|
| pP4-c2 | ggcgaggggggaaagcaccgcgcgcaaaccgacaagttagttaattatttgtgtagt | pP4-c1 |
| | caaagtgccttcagtacatacctcgttaatacattggagcataatgaagaaaatctatgg | |
| | cctatggtCCAAAACTGTCTTTTTTGATGGCACTATCCTG | |
| | AAAAATATGCAAAAAATAGATTGATGTAAGGTGGTT | |
| | CTTGTCAGTGTCGCAAGATCCTTAAGAATTCGTGGC | |
| | ATGAGAGAGTTAAAGGGCGTGCGTTCCTGTATTTCC | |
| | GGAGGGCCCTAATACTCGAATGGTGCAAAACCTTTC | |
| | GCGGTATGGCATGATAGCGCCGAATTCGAAAGAGG | |
| | AGAAAGGAGATnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnntaaCCCGGGCTCGGTACCAAATTCCAGAAAA | |
| | GAGGCCTCCCGAAAGGGGGGCCTTTTTTCGTTTTGGT | |
| | CCGAGCTCCAATTCCGACGTCTAAGAAACCATTATT | |
| | ATCATGACATTAACCTATAAAAATAGGCGTATCACG | |
| | AGGCCCTTTCGTCTTCACCTCGAGTCCCTATCAGTGA | |
| | TAGAGATTGACATCCCTATCAGTGATAGAGATACTG | |
| | AGCACATCAGCAGGACGCACTGACCCCATGGTCTAG | |
| | AGAAAGAGGAGAAAGGAGATATGGAgACCCGATTC | |
| | CCTCAGCAATCGCAGCAAACTCCGGCATCTACCAACA | |
| | GACGCCGGCCATTCAAgCATGAGGATTACCCATGTC | |
| | GAAGACAACAAAGAAGTTCAACTCTTTATGTATTGA | |
| | TCTTCCTCGCGATCTTTCTCTCGAAATTTACCAATCA | |
| | ATTGCTTCTGTCGCTACTGGAAGCGGTGATCCGCAC | |
| | AGTGACGACTTTACAGCAATTGCTTACTTAAggTAAA | |
| | GAGGgGAAAGGAGATATGGTACGCTGGACTTTGTGG | |
| | GATACCCTCGCTTTCCTcCTgtTGctcAGTTTATTGCTGC | |
| | CGTCATTGCTgATcATGTTCATCCCGTCAACATTCAA | |
| | ACGGCCTGTCTCATCATGGAAGGCGCTGAATTTACG | |
| | GAAAACActgTTAATGGCGTCGAGCGTCCGGctgAAGC | |
| | CGCTGAATTGTTCGCGTTTACCTTGCGTGTACGCGCA | |
| | GGAAACACTGACGTTCTTACTGACGCAGAAGAAAAC | |
| | GTGCGTCAAAAATTACGTGCGGAAGGAGTGAtgtaAT | |
| | GAAGATGCCAGAAAAACATGACCTGTTGGCCGCCAT | |
| | TCTCGCGGCAAAGGAACAAGGCATCGGGGCAATCCT | |
| | TGCGTTTGCAATGGCGTACCTTCGCGGCAGATATAA | |
| | TGGCGGTGCGTTTACAAAAACAGTAATCGACGCAAC | |
| | GATGTGCGCCATTATCGCCTGGTTCATTCGTGACCTT | |
| | CTCGACTTCGCCGGACTAAGTAGCAATCTCGCTTAT | |
| | ATAACGAGCGTGTTTATCGGCTACATCGGTACTGAC | |
| | TCGATTGGTTCGCTTATCAAACGCTTCGCTGCTAAAA | |
| | AAGCCGGAGTAGAAGATGGTAGAAATCAATAATCA | |
| | ACGTAAGGCGTTCCTCGATATGCTGGCGTGGTCGGA | |
| | GGGAACTGATAACGGACGTCAGAAAACCAGAAATC | |
| | ATGGTTATGACGTCATTGTAGGCGGAGAGCTATTTA | |
| | CTGATTACTCCGATCACCCTCGCAAACTTGTCACGCT | |
| | AAACCCAAAACTCAAATCAACAGGCGCCGGACGCT | |
| | ACCAGCTTCTTTCCCGTTGGTGGGATGCCTACCGCAA | |
| | GCAGCTTGGCCTGAAAGACTTCTCTCCGAAAAGTCA | |
| | GGACGCTGTGGCATTGCAGCAGATTAAGGAGCGTGG | |
| | CGCTTTACCTATGATTGATCGTGGTGATATCCGTCAG | |
| | GCAATCGACCGTTGCAGCAATATCTGGGCTTCACTG | |
| | CCGGGCGCTGGTTATGGTCAGTTCGAGCATAAGGCT | |
| | GACAGCCTGATTGCAAAATTCAAAGAAGCGGGCGG | |
| | AACGGTCAGAGAGATTGATGTATGAGCAGAGTCACC | |
| | GCGATTATCTCCGCTCTGGTTATCTGCATCATCGTCT | |
| | GCCTGTCATGGGCTGTTAATCATTACCGTGATAACG | |
| | CCATTACCTACAAAGCCCAGCGCGACAAAAATGCCA | |
| | GAGAACTGAAGCTGGCGAACGCGGCAATTACTGAC | |
| | ATGCAGATGCGTCAGCGTGATGTTGCTGCGCTCGAT | |
| | GCAAAATACACGAAGGAGTTAGCTGATGCTAAAGCT | |
| | GAAAATGATGCTCTGCGTGATGATGTTGCCGCTGGT | |
| | CGTCGTCGGTTGCACATCAAAGCAGTCTGTCAGTCA | |
| | GTGCGTGAAGCCACCACCGCCTCCGGCGTGGATAAT | |
| | GCAGCCTCCCCCCGACTGGCAGACACCGCTGAACGG | |
| | GATTATTTCACCCTCAGAGAGAGGCTGATCACTATG | |
| | CAAAAACAACTGGAAGGAACCCAGAAGTATATTAA | |
| | TGAGCAGTGCAGATAActagcataaccccttggggcctctaaacgggt | |
| | cttgaggggttttttgagacaaacaaaagaatggaatcaaagttaaTGCTAGCA | |
| | GCcGCCGctgcaggcatgcaagcttgcggccgcgtcgtgactgggaaaaccct | |
| | ggcgactagtcttggactcctgttgatagatccagtaatgacctcagaactccatctgga | |
| | tttgttcagaacgctcggttgccgccgggcgttttttattggtgagaatccaggggtccc | |
| | caataattacgatttaaatttgtgtctcaaaatctctgatgttacattgcacaagataaaaat | |
| | atatcatcatgaacaataaaactgtctgcttacataaacagtaatacaaggggtgttatga | |
| | gccatattcagcgtgaaacgagctgtagccgtccgcgtctgaacagcaacatggatgc | |
| | ggatctgtatggctataaatgggcgcgtgataacgtgggtcagagcggcgcgaccatt | |
| | tatcgtctgtatggcaaaccggatgcgccggaactgtttctgaaacatggcaaaggca | |
| | gcgtggcgaacgatgtgaccgatgaaatggtgcgtctgaactggctgaccgaatttat | |
| | gccgctgccgaccattaaacattttattcgcaccccggatgatgcgtggctgctgacca | |
| | ccgcgattccgggcaaaaccgcgtttcaggtgctggaagaatatccggatagcggcg | |
| | aaaacattgtggatgcgctggccgtgtttctgcgtcgtctgcatagcattccggtgtgca | |
| | actgcccgtttaacagcgatcgtgtgtttcgtctggcccaggcgcagagccgtatgaac | |
| | aacggcctggtggatgcgagcgattttgatgatgaacgtaacggctggccggtggaa | |
| | caggtgtggaaagaaatgcataaactgctgccgtttagcccggatagcgtggtgaccc | |
| | acggcgattttagcctggataacctgattttcgatgaaggcaaactgattggctgcattg | |
| | atgtgggccgtgtgggcattgcggatcgttatcaggatctggccattctgtggaactgc | |
| | ctgggcgaatttagcccgagcctgcaaaaacgtctgtttcagaaatatggcattgataat | |
| | ccggatatgaacaaactgcaatttcatctgatgctggatgaatttttctaacttggaagtaa | |
| | gaatggtgccgaaggccggactcaaacatcaaaataagttaatgataaaaaacaaata | |
| | ataaaacacaacaatgaaatatgcccccttttgtgcccccactgtttttctgaccaatctat | |
| | tttcagcccatcaataaatcggaaagttaaatcatttttaatcagtaagtttggatccgtag | |
| | ctcggatccaaaccagtgcatcttttatccacataaaaaattttttttcgaaagaactgttca | |
| | cactgttcacctttctgttttctccttttatttcagagtgataggtggtgaataatgggtgaag | |
| | ggtgaacattcgattcttcacctccggcattctgccgatgtgactcataccggtgattaat | |
| | cctccgcactgaaatcactcaggaagaaaaaagttttttttgatttgattgttcacactgttc | |
| | acctttcgtttttctcttttaatttcagtgtgataacgggtgaatatacggtgaagggtgaac | |
| | agtggattgttcaccttcgggggatatcgggataaaaaaagaccggcagatgccggtc | |
| | aggtgggtcaggctgttgtagggtcgtcacattttggcagccagtcgccgtagctttcct | |
| | ctttcagcgtcaggttggtctgtatcccctgtttggtatggcgtttctcgtaattcagtccgt | |
| | attccttcagcatcaccggcagccccagcccgaacattttcagactgagtacattccggt | |
| | agccgtttgcctccatgtaggccagataggcgtgatagaggtatttacggtaattgcgc | |
| | gggatgatactggcgttccccatatacatgccgctggtctgcggcagggtttccagata | |
| | gccgataaaatcaaacgtcgggtcggcatcccgtttgatgttcagtgcctcgtctgagtt | |
| | ctgctgggactgaagcagtgaccgggcgagcatcgggtcgctgaacttctgcatcag | |
| | gtgacgcacgatgaccgccagctcgcgggtgattttgtccttaagctgcgggtcgcgc | |
| | tcctgcggggctatctgttccgggaagtgaataatcacccgtcggcgtgacacgccgc | |
| | cgctgcggtcggtgaagcgcatcgggttattgttcacggccagaatcaccgccgggat | |
| | gtgcgtggagtacgcatcccggtatttcgggtcaacggacaccgcatcgccgccggt | |
| | gatggccttgagtccggcaccgtcgccgctccatttttcctggtccggcaggcgtatca | |
| | gtgagaagccagttaacgcggcacgttcacgcggggattccagcgtctcgatggtgg | |
| | ccgacgtggcgttatcctccccggccagcagggtggctatttcggccatgatacttttgc | |
| | cgctgccgccgggaccggtcacctccagaaagagctgccagtcgtagcggtttgcca | |
| | gcaccataaacagtgcagccagaatcacgtcgcgtttttccgcacggccaccggcgg | |
| | cacggtcaagccagcgccagaaggcgggggcgtgggtttccagcgtttcaccgtcca | |
| | ccggcggggtgaaatccacatcgcacagggtgcgcatccagtgtgacggactgtgc | |
| | gggtggaacgtgccgttctgcgtgtcgagcacgccgttacgaaagccaatcaggcgg | |
| | cgggagggggcttcctgctgcggaataatcagcttcagggtatccaccacggaggcc | |
| | accttcccggaggagaacggcgcacgcagacgctgaaacagcccggccacatccc | |
| | gggcaaagtcctgtggcggcagcaccttccagacaccattttcatagcgggacagaa | |
| | gctggccgttggcatcgaccgcgagcgcctcgccgtaatgctcatagatacgcatggc | |
| | cttttcgctggtactcatggcggaaaactccgcttcgctcatggtgtcgaacgggctttc | |
| | agccggtggccggatggcatcgtaaatggccttacgggtggcctccccgccgtactg | |
| | cgtgaaggcatcattccagtcaccgaagaccggcggcagggcaacaacaccttcaca | |
| | cgcatctgcggctgcggcggcttttttctggccgtcaccactgaggtcacggtcagcg | |
| | gcaaggacaatctgacaggcggggtgcttctgccgggcaaggctggccagagaaag | |
| | gaggttcacggaagaaagcgccaccatcaccgtttcaccggtcaggtgatgtacggta | |
| | agtgcggtcgcgtatccctccgctatccacagacgttttccggcctgattctgtccttcaa | |
| | gggtgtgacaggtgcccctgacctgtccgcctttcagggtgcgcttacggccgtcagc | |
| | actgattaactgaaggttaaccagttcgccgctgtcgtcatacagtggcaccacaaggt | |
| | caccggcgcgccagctcacgccaccggctctgtgtgtgccggtcagcatccggcatt | |
| | cccggccgggaaagcccttgcgggtcaggtaggcgttaccggttccggtacgggtttt | |
| | cgccatcagggtttgtgccagtgcggcggcgttcttccgggcagcgtctgtttcatcaa | |
| | cggcggcggtcgtcactgccgggtcagccggtggcaggctgccggtcacggcagc | |
| | cacctttgcggccgcgtcggacggggaaacaccaaaaaccttttcaaccagtttcagg | |
| | ccgtcaccggcaccacactgattgcagtaccaggtgccgcgcccctccctgtcatcaa | |
| | aacggaagcggtcactcccgccacagaccggacagggctgatgacggttcttcagca | |
| | cctgaatccccagcgccgggagaatacgcggccagtggccgagcgcatggctgac | |
| | ggtggcggttacgttcattttcatggtgttgttctccttcagtgcagtaccggcgcttttatg | |
| | tgacgggcacagagttcatccatcacaaccagcccgagaaaggacagcgacggcgc | |
| | ggccttcagggggccggattccattaaatcttccagcagggcacaggctatctgacgc | |
| | cctttttcctcaccgtgctggcgcagataaaagccttccagctcagcggcgatggccgc | |
| | ctccagtgactcaagggtgagatgcgggtagcggtgctgacgttcgcacacggtcag | |
| | ccaggcacaggcgacagcgcgacggtaaagggcagcgcgtaagacgggcggtaa | |
| | gggtgttttcatttgcttttctccctgtgacagatgactgcattccgtgccggttgcattaac | |
| | tgataaggcatatctgcgtctcctgaagacgtgcgtatccctgcgcgaatacgcacattt | |
| | aatttttcgggggtcgttttttaattacagataattgcggtaactgttatccggggtggtttc | |
| | cgggtcaggctccgtgcggggaatttcccgccattcccgcgccaccggtgctgcccg | |
| | gctgaccggaacagtgtcctgcgggtaaatatccagatatttttcccgccatttctgtaatt | |
| | ccgggtctccggccatttctttcagtaccgcatgccggtttacggggctgcgtttaaaca | |
| | ggtcaggacggtcacaggtaaattcccgcagaaaacgccccagcgggatgtctgtgg | |
| | tgcgtccgtcagcgaggatacgcacaaggatactgaatttacggcggtacgggttcca | |
| | gacaatgtccgggcagcggtacggcatttcccacggaataccgtcttccagaatgccg | |
| | accacggccacatcgggaaaaccggcagaacggtaaatctcaccgggctggggaa | |
| | aatcaaacatgcgtcctgtctccccggtctttctgctgggcgagaaaatcgcggcacag | |
| | gcctttggctttcagctcattcagcacaaaatcaatatcttcattcaggtagctgaaaatat | |
| | gtggaatgtagagctgatgcaggccggagagttcacggtgaatcaaatcacccccaa | |
| | caaaccgggatacggcgctggcgcggttgagcttatggtaagcctcaatgctgaggtg | |
| | ttcacgggcgtcatgacgcgctgagacggtctgaggggcttttttattacgcacgggac | |
| | acctccaccaccggcagacgggcagcaagggagagcacatagtcacggacaaggg | |
| | aacggcgggcactgcgttcatcaccggcgacggtgcgaagcatacagatacgggg | |
| | tgacggtctgcgcgacggacagccgcaaacacaaagacaaattcagggtgtgaggg | |
| | ggtaagggttgtagccatgatggcagcctcctgtgaatagcaaataacgctatcgccg | |
| | gagttctcacgctcgatggcgatagcccagacgggggtgagaataccggcttcacag | |
| | gataccggccagcccggaggctgccccgcctgagctaccattgactctgcggcataa | |
| | tgagcggacgcgggcaggatgcacggaatgccatctgcacgactgaccacacacca | |
| | caccataatctggcgctctgtggcattgattgcgacacaaaaaaagacgcgtggcgcg | |
| | tcatatgtcgcctgtgaattgctcgggttctcacgcccggctgccgattttgcggcaggc | |
| | gaaaaactatatccgcaaatgccggaaaaaggcaagccagaaaaagggagtttttgc | |
| | agagcgggcatcatcatgcgtcgtacccccgtttgcgtccggcaatgcgtccggccat | |
| | ccatgcggtgacttcagagtgcagccaggccacatttttaccgccaagactcacctgc | |
| | ggcggaaattcccccttacggatgagttcgtagatggtcgagcgtgacaggccgcac | |
| | aggtgcatcacttccggcacacgtaaaaaacgctcctgcgtgatgtccggcagcggc | |
| | atcagtggcgtcactggggcgggagacggggaagaaaaaacagcttgcatcgggct | |
| | acctcgttaatgtccatacagcaccggataagtccgtccggcttcgggtagcgctttattt | |
| | tgtgaatattttcagcagacgcaacaggggggatttgttcaggctgtcttacaatggctgt | |
| | gtgttttttgttcatctccacttaaagtcatttaaagccacttaaagcaatttgtaatttttatag | |
| | tgaaatacaaatcgtttcttcttattcattcccggcgaattaataaaaacaaacagtagtaa | |
| | acagcacaaaaagcccatcaacgggtgaacagtggtgaacagacggtgaacagtca | |
| | ttactgcgattgttcaccctttaacttactgtattacttatcttttttattaaggtgaacagagg | |
| | tgaacagtaaaatataaaaaaacaaacagtaagccggtttttcctgcgaccttttcctgg | |
| | cttgccggtctgaggatgagtctcctgtgtcagggctggcacatctgcaatgcgtcgtgt | |
| | tgttgtccggtgtacgtcacaattttcttaacctgaagtgacgaggagccggaaaatgtc | |
| | tgaccacactatccctgaatatctgcaacccgcactggcacaactggaaaaggccaga | |
| | gccgcccatcttgagaacgcccgcctgatggatgagaccgtcacggccattgaacgg | |
| | gcagagcaggaaaaaaatgcgctggcgcaggccgacggaaacgacgctgacgact | |
| | ggcgcacggcctttcgtgcagccggtggtgtcctgagcgacgagctgaaacagcgc | |
| | cacattgagcgcgtggcacgccgggagctggtacaggaatatgacaatctggccgtg | |
| | gtgctgaatttcgaacgtgaacgcctgaaaggggcgtgtgacagcacggccaccgcc | |
| | taccggaaggcacatcatcaccttctgagtctgtatgcagagcatgagctggaacacg | |
| | ccctgaatgaaacctgtgaggcgcttgtccgggcaatgcatctgagcattctggtacag | |
| | gaaaatccgctcgccaacaccaccggccatcagggctacgtcgcaccggaaaaggc | |
| | tgtcatgcagcaggtgaaatcatcgctggaacagaaaattaaacagatgcaaatcagc | |
| | ctcaccggcgagccggttctccggctgaccggactgtcagcggcaacactcccgcac | |
| | atggattatgaggtggcaggcacaccggcacagcgcaaggtgtggcaggacaaaat | |
| | agaccagcagggagcagagcttaaggccagagggctgctgtcatgatttactgtccgt | |
| | cgtgtggacatgttgctcacacccgtcgcgcacatttcatggacgatggcaccaagata | |
| | atgattgcacagtgccggaatatttattgctctgcgacatttgaagcgagtgaaagcttttt | |
| | ctctgacagtaaagattcaggaatggaatacatttcaggcaaacagagataccgcgatt | |
| | cactgacgtcagcctcctgcggtatgaaacgcccgaaaagaatgcttgttaccggatat | |
| | tgttgtcggagatgtaaaggccttgcactgtcaagaacatcgcggcgtctgtctcagga | |
| | agtcaccgagcgtttttatgtgtgcacggatccgggctgtggtctggtgtttaaaacgctt | |
| | cagaccatcaaccgcttcattgtccgcccggtcacgccggacgaactggcagaacgc | |
| | ctgcatgaaaaacaggaactgccgccagtacggttaaaaacacaatcatattcgctgc | |
| | gtctggaatgagggctgccggttaacaccggccgtcgccgcacaccgtatttttattctt | |
| | cagcatgatgagaaagagataacgatggaaagcacagccttacagcaggcctttgac | |
| | acctgtcagaataacaaagcagcatggctgcaacgcaaaaatgagctggcagcggc | |
| | cgaacaggaatatctgcggcttctgtcaggagaaggcagaaacgtcagtcgcctgga | |
| | cgaattacgcaatattatcgaagtcagaaaatggcaggtgaatcaggccgccggtcgtt | |
| | atattcgttcgcatgaagccgttcagcacatcagcatccgcgaccggctgaatgatttta | |
| | tgcagcagcacggcacagcactggcggccgcactggcaccggagctgatgggcta | |
| | cagtgagctgacggccattgcccgaaactgtgccatacagcgtgccacagatgccct | |
| | gcgtgaagcccttctgtcctggcttgcgaagggtgaaaaaattaattattccgcacagg | |
| | atagcgacattttaacgaccatcggattcaggcctgacgtggcttcggtggatgacagc | |
| | cgtgaaaaattcacccctgcgcagaacatgattttttcgcgtaaaagtgcgcaactggc | |
| | atcacgtcagtcagtgtaaaattccccgaaaatccgcccgtttttactgaaaaaagccat | |
| | gcatcgataaggtgcatggctttgcatgcgttttcctgcctcattttctgcaaaccgcgcc | |
| | attcccggcgcggtctgagcgtgtcagtgcaactgcattaaaaccgccccgcaaagc | |
| | gggcg | |
| pP4- | ggcgaggcggggaaagcaccgcgcgcaaaccgacaagttagttaattatttgtgtagt | pP4-c2 |
| c2Δlys | caaagtgccttcagtacatacctcgttaatacattggagcataatgaagaaaatctatgg | |
| | cctatggtCCAAAACTGTCTTTTTTGATGGCACTATCCTG | |
| | AAAAATATGCAAAAAATAGATTGATGTAAGGTGGTT | |
| | CTTGTCAGTGTCGCAAGATCCTTAAGAATTCGTGGC | |
| | ATGAGAGAGTTAAAGGGCGTGCGTTCCTGTATTTCC | |
| | GGAGGGCCCTAATACTCGAATGGTGCAAAACCTTTC | |
| | GCGGTATGGCATGATAGCGCCGAATTCGAAAGAGG | |
| | AGAAAGGAGATnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnntaaCCCGGGCTCGGTACCAAATTC | |
| | CAGAAAAGAGGCCTCCCGAAAGGGGGGCCTTTTTTC | |
| | GTTTTGGTCCctagcataaccccttggggcctctaaacgggtcttgaggggttt | |
| | tttgagacaaacaaaagaatggaatcaaagttaaTGCTAGCAGCcGCCGc | |
| | tgcaggcatgcaagcttgcggccgcgtcgtgactgggaaaaccctggcgactagtctt | |
| | ggactcctgttgatagatccagtaatgacctcagaactccatctggatttgttcagaacg | |
| | ctcggttgccgccgggcgttttttattggtgagaatccaggggtccccaataattacgatt | |
| | taaatttgtgtctcaaaatctctgatgttacattgcacaagataaaaatatatcatcatgaac | |
| | aataaaactgtctgcttacataaacagtaatacaaggggtgttatgagccatattcagcgt | |
| | gaaacgagctgtagccgtccgcgtctgaacagcaacatggatgcggatctgtatggct | |
| | ataaatgggcgcgtgataacgtgggtcagagcggcgcgaccatttatcgtctgtatgg | |
| | caaaccggatgcgccggaactgtttctgaaacatggcaaaggcagcgtggcgaacg | |
| | atgtgaccgatgaaatggtgcgtctgaactggctgaccgaatttatgccgctgccgacc | |
| | attaaacattttattcgcaccccggatgatgcgtggctgctgaccaccgcgattccggg | |
| | caaaaccgcgtttcaggtgctggaagaatatccggatagcggcgaaaacattgtggat | |
| | gcgctggccgtgtttctgcgtcgtctgcatagcattccggtgtgcaactgcccgtttaac | |
| | agcgatcgtgtgtttcgtctggcccaggcgcagagccgtatgaacaacggcctggtgg | |
| | atgcgagcgattttgatgatgaacgtaacggctggccggtggaacaggtgtggaaag | |
| | aaatgcataaactgctgccgtttagcccggatagcgtggtgacccacggcgattttagc | |
| | ctggataacctgattttcgatgaaggcaaactgattggctgcattgatgtgggccgtgtg | |
| | ggcattgcggatcgttatcaggatctggccattctgtggaactgcctgggcgaatttagc | |
| | ccgagcctgcaaaaacgtctgtttcagaaatatggcattgataatccggatatgaacaa | |
| | actgcaatttcatctgatgctggatgaatttttctaacttggaagtaagaatggtgccgaa | |
| | ggccggactcaaacatcaaaataagttaatgataaaaaacaaataataaaacacaaca | |
| | atgaaatatgcccccttttgtgcccccactgtttttctgaccaatctattttcagcccatcaa | |
| | taaatcggaaagttaaatcatttttaatcagtaagtttggatccgtagctcggatccaaac | |
| | cagtgcatcttttatccacataaaaaattttttttcgaaagaactgttcacactgttcaccttt | |
| | ctgttttctccttttatttcagagtgataggtggtgaataatgggtgaagggtgaacattcg | |
| | attcttcacctccggcattctgccgatgtgactcataccggtgattaatcctccgcactga | |
| | aatcactcaggaagaaaaaagttttttttgatttgattgttcacactgttcacctttcgtttttc | |
| | tcttttaatttcagtgtgataacgggtgaatatacggtgaagggtgaacagtggattgttc | |
| | accttcgggggatatcgggataaaaaaagaccggcagatgccggtcaggtgggtca | |
| | ggctgttgtagggtcgtcacattttggcagccagtcgccgtagctttcctctttcagcgtc | |
| | aggttggtctgtatcccctgtttggtatggcgtttctcgtaattcagtccgtattccttcagc | |
| | atcaccggcagccccagcccgaacattttcagactgagtacattccggtagccgtttgc | |
| | ctccatgtaggccagataggcgtgatagaggtatttacggtaattgcgcgggatgatac | |
| | tggcgttccccatatacatgccgctggtctgcggcagggtttccagatagccgataaaa | |
| | tcaaacgtcgggtcggcatcccgtttgatgttcagtgcctcgtctgagttctgctgggact | |
| | gaagcagtgaccgggcgagcatcgggtcgctgaacttctgcatcaggtgacgcacga | |
| | tgaccgccagctcgcgggtgattttgtccttaagctgcgggtcgcgctcctgcggggct | |
| | atctgttccgggaagtgaataatcacccgtcggcgtgacacgccgccgctgcggtcg | |
| | gtgaagcgcatcgggttattgttcacggccagaatcaccgccgggatgtgcgtggagt | |
| | acgcatcccggtatttcgggtcaacggacaccgcatcgccgccggtgatggccttgag | |
| | tccggcaccgtcgccgctccatttttcctggtccggcaggcgtatcagtgagaagcca | |
| | gttaacgcggcacgttcacgcggggattccagcgtctcgatggtggccgacgtggcg | |
| | ttatcctccccggccagcagggtggctatttcggccatgatacttttgccgctgccgccg | |
| | ggaccggtcacctccagaaagagctgccagtcgtagcggtttgccagcaccataaac | |
| | agtgcagccagaatcacgtcgcgtttttccgcacggccaccggcggcacggtcaagc | |
| | cagcgccagaaggcgggggcgtgggtttccagcgtttcaccgtccaccggcggggt | |
| | gaaatccacatcgcacagggtgcgcatccagtgtgacggactgtgcgggtggaacgt | |
| | gccgttctgcgtgtcgagcacgccgttacgaaagccaatcaggcggcgggaggggg | |
| | cttcctgctgcggaataatcagcttcagggtatccaccacggaggccaccttcccgga | |
| | ggagaacggcgcacgcagacgctgaaacagcccggccacatcccgggcaaagtcc | |
| | tgtggcggcagcaccttccagacaccattttcatagcgggacagaagctggccgttgg | |
| | catcgaccgcgagcgcctcgccgtaatgctcatagatacgcatggccttttcgctggta | |
| | ctcatggcggaaaactccgcttcgctcatggtgtcgaacgggctttcagccggtggcc | |
| | ggatggcatcgtaaatggccttacgggtggcctccccgccgtactgcgtgaaggcatc | |
| | attccagtcaccgaagaccggcggcagggcaacaacaccttcacacgcatctgcggc | |
| | tgcggcggcttttttctggccgtcaccactgaggtcacggtcagcggcaaggacaatct | |
| | gacaggcggggtgcttctgccgggcaaggctggccagagaaaggaggttcacgga | |
| | agaaagcgccaccatcaccgtttcaccggtcaggtgatgtacggtaagtgcggtcgcg | |
| | tatccctccgctatccacagacgttttccggcctgattctgtccttcaagggtgtgacagg | |
| | tgcccctgacctgtccgcctttcagggtgcgcttacggccgtcagcactgattaactga | |
| | aggttaaccagttcgccgctgtcgtcatacagtggcaccacaaggtcaccggcgcgc | |
| | cagctcacgccaccggctctgtgtgtgccggtcagcatccggcattcccggccggga | |
| | aagcccttgcgggtcaggtaggcgttaccggttccggtacgggttttcgccatcagggt | |
| | ttgtgccagtgcggcggcgttcttccgggcagcgtctgtttcatcaacggcggcggtcg | |
| | tcactgccgggtcagccggtggcaggctgccggtcacggcagccacctttgcggcc | |
| | gcgtcggacggggaaacaccaaaaaccttttcaaccagtttcaggccgtcaccggca | |
| | ccacactgattgcagtaccaggtgccgcgcccctccctgtcatcaaaacggaagcggt | |
| | cactcccgccacagaccggacagggctgatgacggttcttcagcacctgaatcccca | |
| | gcgccgggagaatacgcggccagtggccgagcgcatggctgacggtggcggttac | |
| | gttcattttcatggtgttgttctccttcagtgcagtaccggcgcttttatgtgacgggcaca | |
| | gagttcatccatcacaaccagcccgagaaaggacagcgacggcgcggccttcaggg | |
| | ggccggattccattaaatcttccagcagggcacaggctatctgacgccctttttcctcac | |
| | cgtgctggcgcagataaaagccttccagctcagcggcgatggccgcctccagtgact | |
| | caagggtgagatgcgggtagcggtgctgacgttcgcacacggtcagccaggcacag | |
| | gcgacagcgcgacggtaaagggcagcgcgtaagacgggcggtaagggtgttttcatt | |
| | tgcttttctccctgtgacagatgactgcattccgtgccggttgcattaactgataaggcat | |
| | atctgcgtctcctgaagacgtgcgtatccctgcgcgaatacgcacatttaatttttcgggg | |
| | gtcgttttttaattacagataattgcggtaactgttatccggggtggtttccgggtcaggct | |
| | ccgtgcggggaatttcccgccattcccgcgccaccggtgctgcccggctgaccggaa | |
| | cagtgtcctgcgggtaaatatccagatatttttcccgccatttctgtaattccgggtctccg | |
| | gccatttctttcagtaccgcatgccggtttacggggctgcgtttaaacaggtcaggacg | |
| | gtcacaggtaaattcccgcagaaaacgccccagcgggatgtctgtggtgcgtccgtca | |
| | gcgaggatacgcacaaggatactgaatttacggcggtacgggttccagacaatgtccg | |
| | ggcagcggtacggcatttcccacggaataccgtcttccagaatgccgaccacggcca | |
| | catcgggaaaaccggcagaacggtaaatctcaccgggctggggaaaatcaaacatg | |
| | cgtcctgtctccccggtctttctgctgggcgagaaaatcgcggcacaggcctttggcttt | |
| | cagctcattcagcacaaaatcaatatcttcattcaggtagctgaaaatatgtggaatgtag | |
| | agctgatgcaggccggagagttcacggtgaatcaaatcacccccaacaaaccgggat | |
| | acggcgctggcgcggttgagcttatggtaagcctcaatgctgaggtgttcacgggcgt | |
| | catgacgcgctgagacggtctgaggggcttttttattacgcacgggacacctccaccac | |
| | cggcagacgggcagcaagggagagcacatagtcacggacaagggaacggcgggc | |
| | actgcgttcatcaccggcgacggtgcgaagcatacagatacggggatgacggtctgc | |
| | gcgacggacagccgcaaacacaaagacaaattcagggtgtgagggggtaagggttg | |
| | tagccatgatggcagcctcctgtgaatagcaaataacgctatcgccggagttctcacgc | |
| | tcgatggcgatagcccagacgggggtgagaataccggcttcacaggataccggcca | |
| | gcccggaggctgccccgcctgagctaccattgactctgcggcataatgagcggacgc | |
| | gggcaggatgcacggaatgccatctgcacgactgaccacacaccacaccataatctg | |
| | gcgctctgtggcattgattgcgacacaaaaaaagacgcgtggcgcgtcatatgtcgcc | |
| | tgtgaattgctcgggttctcacgcccggctgccgattttgcggcaggcgaaaaactatat | |
| | ccgcaaatgccggaaaaaggcaagccagaaaaagggagtttttgcagagcgggcat | |
| | catcatgcgtcgtacccccgtttgcgtccggcaatgcgtccggccatccatgcggtgac | |
| | ttcagagtgcagccaggccacatttttaccgccaagactcacctgcggcggaaattccc | |
| | ccttacggatgagttcgtagatggtcgagcgtgacaggccgcacaggtgcatcacttc | |
| | cggcacacgtaaaaaacgctcctgcgtgatgtccggcagcggcatcagtggcgtcac | |
| | tggggcgggagacggggaagaaaaaacagcttgcatcgggctacctcgttaatgtcc | |
| | atacagcaccggataagtccgtccggcttcgggtagcgctttattttgtgaatattttcag | |
| | cagacgcaacaggggggatttgttcaggctgtcttacaatggctgtgtgttttttgttcatc | |
| | tccacttaaagtcatttaaagccacttaaagcaatttgtaatttttatagtgaaatacaaatc | |
| | gtttcttcttattcattcccggcgaattaataaaaacaaacagtagtaaacagcacaaaaa | |
| | gcccatcaacgggtgaacagtggtgaacagacggtgaacagtcattactgcgattgtt | |
| | caccctttaacttactgtattacttatcttttttattaaggtgaacagaggtgaacagtaaaat | |
| | ataaaaaaacaaacagtaagccggtttttcctgcgaccttttcctggcttgccggtctga | |
| | ggatgagtctcctgtgtcagggctggcacatctgcaatgcgtcgtgttgttgtccggtgt | |
| | acgtcacaattttcttaacctgaagtgacgaggagccggaaaatgtctgaccacactat | |
| | ccctgaatatctgcaacccgcactggcacaactggaaaaggccagagccgcccatctt | |
| | gagaacgcccgcctgatggatgagaccgtcacggccattgaacgggcagagcagg | |
| | aaaaaaatgcgctggcgcaggccgacggaaacgacgctgacgactggcgcacggc | |
| | ctttcgtgcagccggtggtgtcctgagcgacgagctgaaacagcgccacattgagcg | |
| | cgtggcacgccgggagctggtacaggaatatgacaatctggccgtggtgctgaatttc | |
| | gaacgtgaacgcctgaaaggggcgtgtgacagcacggccaccgcctaccggaagg | |
| | cacatcatcaccttctgagtctgtatgcagagcatgagctggaacacgccctgaatgaa | |
| | acctgtgaggcgcttgtccgggcaatgcatctgagcattctggtacaggaaaatccgct | |
| | cgccaacaccaccggccatcagggctacgtcgcaccggaaaaggctgtcatgcagc | |
| | aggtgaaatcatcgctggaacagaaaattaaacagatgcaaatcagcctcaccggcg | |
| | agccggttctccggctgaccggactgtcagcggcaacactcccgcacatggattatga | |
| | ggtggcaggcacaccggcacagcgcaaggtgtggcaggacaaaatagaccagcag | |
| | ggagcagagcttaaggccagagggctgctgtcatgatttactgtccgtcgtgtggacat | |
| | gttgctcacacccgtcgcgcacatttcatggacgatggcaccaagataatgattgcaca | |
| | gtgccggaatatttattgctctgcgacatttgaagcgagtgaaagctttttctctgacagta | |
| | aagattcaggaatggaatacatttcaggcaaacagagataccgcgattcactgacgtca | |
| | gcctcctgcggtatgaaacgcccgaaaagaatgcttgttaccggatattgttgtcggag | |
| | atgtaaaggccttgcactgtcaagaacatcgcggcgtctgtctcaggaagtcaccgag | |
| | cgtttttatgtgtgcacggatccgggctgtggtctggtgtttaaaacgcttcagaccatca | |
| | accgcttcattgtccgcccggtcacgccggacgaactggcagaacgcctgcatgaaa | |
| | aacaggaactgccgccagtacggttaaaaacacaatcatattcgctgcgtctggaatga | |
| | gggctgccggttaacaccggccgtcgccgcacaccgtatttttattcttcagcatgatga | |
| | gaaagagataacgatggaaagcacagccttacagcaggcctttgacacctgtcagaat | |
| | aacaaagcagcatggctgcaacgcaaaaatgagctggcagcggccgaacaggaata | |
| | tctgcggcttctgtcaggagaaggcagaaacgtcagtcgcctggacgaattacgcaat | |
| | attatcgaagtcagaaaatggcaggtgaatcaggccgccggtcgttatattcgttcgcat | |
| | gaagccgttcagcacatcagcatccgcgaccggctgaatgattttatgcagcagcacg | |
| | gcacagcactggcggccgcactggcaccggagctgatgggctacagtgagctgacg | |
| | gccattgcccgaaactgtgccatacagcgtgccacagatgccctgcgtgaagcccttc | |
| | tgtcctggcttgcgaagggtgaaaaaattaattattccgcacaggatagcgacattttaa | |
| | cgaccatcggattcaggcctgacgtggcttcggtggatgacagccgtgaaaaattcac | |
| | ccctgcgcagaacatgattttttcgcgtaaaagtgcgcaactggcatcacgtcagtcagt | |
| | gtaaaattccccgaaaatccgcccgtttttactgaaaaaagccatgcatcgataaggtgc | |
| | atggctttgcatgcgttttcctgcctcattttctgcaaaccgcgccattcccggcgcggtc | |
| | tgagcgtgtcagtgcaactgcattaaaaccgccccgcaaagcgggcg | |
| pP4- | ggcgaggcggggaaagcaccgcgcgcaaaccgacaagttagttaattatttgtgtagt | pP4-c2Δlys |
| LacIq- | caaagtgccttcagtacatacctcgttaatacattggagcataatgaagaaaatctatgg | |
| p72 | cctatggtCCAAAACTGTCTTTTTTGATGGCACTATCCTG | |
| | AAAAATATGCAAAAAATAGATTGATGTAAGGTGGTT | |
| | CTTGTCAGTGTCGCAAGATCCTTAAGAATTCGTGGC | |
| | ATGAGAGAGTTAAAGGGCGTGCGTTCCTGTATTTCC | |
| | GGAGGGCCCTAATACTCGAATGGTGCAAAACCTTTC | |
| | GCGGTATGGCATGATAGCGCCGAATTCGAAAGAGG | |
| | AGAAAGGAGATcaatgaaaaaggccctggcaacactgattgcactggcact | |
| | gccggcagcagcactggcagaaggtaccgaaaacgtgcagtatcgccacgttgaact | |
| | ggcacgcgttggccaattagttgaggtggacaccctggaacacgtgcagcatattattg | |
| | gcggtgccggcaatgacagcattacaggcaacgcccacgacaactttctggcaggcg | |
| | gcagcggcgatgatcgtctggatggtggcgccggtaatgataccctggtgggtggcg | |
| | agggtcaaaataccgttattggcggtgccggcgacgatgttttcctgcaggatctgggc | |
| | gtgtggagcaaccagctggacggtggtgcaggtgtggacaccgtgaaatacaacgtt | |
| | catcagccgagcgaggaacgtctggaacgcatgggtgacaccggtattcatgccgat | |
| | ctgcagaaaggcaccgtggaaaaatggccggccctgaacctgtttagcgtggatcatg | |
| | ttaaaaatattgaaaatctgcatggcagccgtctgaacgaccgcattgccggcgatgac | |
| | caagataacgaactgtggggccatgacggtaatgacaccattcgcggccgtggtggc | |
| | gacgacattctgcgtggtggtctgggtctggatacactgtacggtgaggatggcaatga | |
| | tatctttctgcaggatgacgaaacagttagcgacgacattgacggtggcgcaggtctgg | |
| | acacagtggactacagcgccatgattcatccgggccgtattgtggccccgcacgaata | |
| | tggttttggtatcgaagccgatctgagccgtgaatgggtgcgcaaagcaagtgccctg | |
| | ggtgtggactattacgacaacgtgcgcaatgtggaaaacgtgattggcaccagcatga | |
| | aagacgtgctgattggcgacgcccaggccaacaccctgatgggccagggtggtgac | |
| | gatacagtgcgcggtggtgatggtgatgacctgctgttcggcggcgatggtaacgaca | |
| | tgctgtatggcgatgccggtaacgacaccctgtacggcggtctgggtgacgataccct | |
| | ggagggcggtgccggcaacgattggtttggccaaacccaggcccgtgaacatgatgt | |
| | gctgcgcggtggcgatggcgtggataccgttgactacagtcagacaggcgcccatgc | |
| | aggtatcgcagccggtcgcattggtctgggtattctggcagacctgggtgccggtcgt | |
| | gtggataaactgggtgaggccggcagtagtgcctatgacacagtgagcggcatcgaa | |
| | aacgtggtgggcacagagctggcagaccgtatcacaggcgatgcccaagccaatgtt | |
| | ctgcgcggcgcaggtggtgccgatgtgttagccggtggtgagggtgatgacgttctgc | |
| | tgggcggtgacggcgatgatcagctgagtggtgatgcaggtcgcgaccgcctgtatg | |
| | gcgaagcaggcgatgattggttctttcaggacgcagccaatgccggtaatctgttagac | |
| | ggtggcgacggtcgtgatacagtggattttagcggtcctggtcgtggcctggatgccg | |
| | gcgcaaagggtgtgtttctgagcctgggcaaaggctttgccagcttaatggacgaacc | |
| | ggaaaccagcaacgtgttacgcaacattgagaacgccgtgggtagtgcccgcgatga | |
| | cgtgctgatcggtgatgccggtgcaaatgttctgaacggcctggccggcaacgacgtg | |
| | ctgagtggtggtgcaggtgatgatgttctgctgggtgatgagggcagtgacttactgag | |
| | cggtgacgccggcaatgacgatttattcggcggccagggtgatgacacctacctgtttg | |
| | gcgtgggttatggccacgacaccatttatgagagcggtggtggtcacgacacaatccg | |
| | catcaatgcaggcgccgatcagctgtggtttgcccgccagggtaacgatctggagatt | |
| | cgtatcctgggcacagacgatgccttaacagtgcatgattggtaccgcgatgccgatca | |
| | tcgcgtggagattatccatgcagcaaaccaggcagtggatcaggccggcatcgagaa | |
| | gctggttgaggccatggcccagtatcctgatcctggtggtcatcatcaccaccatcatta | |
| | ataactcgagnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnntaaCCCGGGCTCGGTACCAA | |
| | ATTCCAGAAAAGAGGCCTCCCGAAAGGGGGGCCTTT | |
| | TTTCGTTTTGGTCCctagcataaccccttggggcctctaaacgggtcttga | |
| | ggggttttttgagacaaacaaaagaatggaatcaaagttaaTGCTAGCAGCc | |
| | GCCGctgcaggcatgcaagcttgcggccgcgtcgtgactgggaaaaccctggcg | |
| | actagtcttggactcctgttgatagatccagtaatgacctcagaactccatctggatttgtt | |
| | cagaacgctcggttgccgccgggcgttttttattggtgagaatccaggggtccccaata | |
| | attacgatttaaatttgtgtctcaaaatctctgatgttacattgcacaagataaaaatatatc | |
| | atcatgaacaataaaactgtctgcttacataaacagtaatacaaggggtgttatgagcca | |
| | tattcagcgtgaaacgagctgtagccgtccgcgtctgaacagcaacatggatgcggat | |
| | ctgtatggctataaatgggcgcgtgataacgtgggtcagagcggcgcgaccatttatc | |
| | gtctgtatggcaaaccggatgcgccggaactgtttctgaaacatggcaaaggcagcgt | |
| | ggcgaacgatgtgaccgatgaaatggtgcgtctgaactggctgaccgaatttatgccg | |
| | ctgccgaccattaaacattttattcgcaccccggatgatgcgtggctgctgaccaccgc | |
| | gattccgggcaaaaccgcgtttcaggtgctggaagaatatccggatagcggcgaaaa | |
| | cattgtggatgcgctggccgtgtttctgcgtcgtctgcatagcattccggtgtgcaactg | |
| | cccgtttaacagcgatcgtgtgtttcgtctggcccaggcgcagagccgtatgaacaac | |
| | ggcctggtggatgcgagcgattttgatgatgaacgtaacggctggccggtggaacag | |
| | gtgtggaaagaaatgcataaactgctgccgtttagcccggatagcgtggtgacccacg | |
| | gcgattttagcctggataacctgattttcgatgaaggcaaactgattggctgcattgatgt | |
| | gggccgtgtgggcattgcggatcgttatcaggatctggccattctgtggaactgcctgg | |
| | gcgaatttagcccgagcctgcaaaaacgtctgtttcagaaatatggcattgataatccgg | |
| | atatgaacaaactgcaatttcatctgatgctggatgaatttttctaacttggaagtaagaat | |
| | ggtgccgaaggccggactcaaacatcaaaataagttaatgataaaaaacaaataataa | |
| | aacacaacaatgaaatatgcccccttttgtgcccccactgtttttctgaccaatctattttca | |
| | gcccatcaataaatcggaaagttaaatcatttttaatcagtaagtttggatccgtagctcg | |
| | gatccaaaccagtgcatcttttatccacataaaaaattttttttcgaaagaactgttcacact | |
| | gttcacctttctgttttctccttttatttcagagtgataggtggtgaataatgggtgaagggt | |
| | gaacattcgattcttcacctccggcattctgccgatgtgactcataccggtgattaatcct | |
| | ccgcactgaaatcactcaggaagaaaaaagttttttttgatttgattgttcacactgttcac | |
| | ctttcgtttttctcttttaatttcagtgtgataacgggtgaatatacggtgaagggtgaacag | |
| | tggattgttcaccttcgggggatatcgggataaaaaaagaccggcagatgccggtcag | |
| | gtgggtcaggctgttgtagggtcgtcacattttggcagccagtcgccgtagctttcctctt | |
| | tcagcgtcaggttggtctgtatcccctgtttggtatggcgtttctcgtaattcagtccgtatt | |
| | ccttcagcatcaccggcagccccagcccgaacattttcagactgagtacattccggtag | |
| | ccgtttgcctccatgtaggccagataggcgtgatagaggtatttacggtaattgcgcgg | |
| | gatgatactggcgttccccatatacatgccgctggtctgcggcagggtttccagatagc | |
| | cgataaaatcaaacgtcgggtcggcatcccgtttgatgttcagtgcctcgtctgagttct | |
| | gctgggactgaagcagtgaccgggcgagcatcgggtcgctgaacttctgcatcaggt | |
| | gacgcacgatgaccgccagctcgcgggtgattttgtccttaagctgcgggtcgcgctc | |
| | ctgcggggctatctgttccgggaagtgaataatcacccgtcggcgtgacacgccgcc | |
| | gctgcggtcggtgaagcgcatcgggttattgttcacggccagaatcaccgccgggatg | |
| | tgcgtggagtacgcatcccggtatttcgggtcaacggacaccgcatcgccgccggtg | |
| | atggccttgagtccggcaccgtcgccgctccatttttcctggtccggcaggcgtatcagt | |
| | gagaagccagttaacgcggcacgttcacgcggggattccagcgtctcgatggtggcc | |
| | gacgtggcgttatcctccccggccagcagggtggctatttcggccatgatacttttgcc | |
| | gctgccgccgggaccggtcacctccagaaagagctgccagtcgtagcggtttgccag | |
| | caccataaacagtgcagccagaatcacgtcgcgtttttccgcacggccaccggcggc | |
| | acggtcaagccagcgccagaaggcgggggcgtgggtttccagcgtttcaccgtccac | |
| | cggcggggtgaaatccacatcgcacagggtgcgcatccagtgtgacggactgtgcg | |
| | ggtggaacgtgccgttctgcgtgtcgagcacgccgttacgaaagccaatcaggcggc | |
| | gggagggggcttcctgctgcggaataatcagcttcagggtatccaccacggaggcca | |
| | ccttcccggaggagaacggcgcacgcagacgctgaaacagcccggccacatcccg | |
| | ggcaaagtcctgtggcggcagcaccttccagacaccattttcatagcgggacagaag | |
| | ctggccgttggcatcgaccgcgagcgcctcgccgtaatgctcatagatacgcatggcc | |
| | ttttcgctggtactcatggcggaaaactccgcttcgctcatggtgtcgaacgggctttca | |
| | gccggtggccggatggcatcgtaaatggccttacgggtggcctccccgccgtactgc | |
| | gtgaaggcatcattccagtcaccgaagaccggcggcagggcaacaacaccttcacac | |
| | gcatctgcggctgcggcggcttttttctggccgtcaccactgaggtcacggtcagcgg | |
| | caaggacaatctgacaggcggggtgcttctgccgggcaaggctggccagagaaagg | |
| | aggttcacggaagaaagcgccaccatcaccgtttcaccggtcaggtgatgtacggtaa | |
| | gtgcggtcgcgtatccctccgctatccacagacgttttccggcctgattctgtccttcaag | |
| | ggtgtgacaggtgcccctgacctgtccgcctttcagggtgcgcttacggccgtcagca | |
| | ctgattaactgaaggttaaccagttcgccgctgtcgtcatacagtggcaccacaaggtc | |
| | accggcgcgccagctcacgccaccggctctgtgtgtgccggtcagcatccggcattc | |
| | ccggccgggaaagcccttgcgggtcaggtaggcgttaccggttccggtacgggttttc | |
| | gccatcagggtttgtgccagtgcggcggcgttcttccgggcagcgtctgtttcatcaac | |
| | ggcggcggtcgtcactgccgggtcagccggtggcaggctgccggtcacggcagcc | |
| | acctttgcggccgcgtcggacggggaaacaccaaaaaccttttcaaccagtttcaggc | |
| | cgtcaccggcaccacactgattgcagtaccaggtgccgcgcccctccctgtcatcaaa | |
| | acggaagcggtcactcccgccacagaccggacagggctgatgacggttcttcagcac | |
| | ctgaatccccagcgccgggagaatacgcggccagtggccgagcgcatggctgacg | |
| | gtggcggttacgttcattttcatggtgttgttctccttcagtgcagtaccggcgcttttatgt | |
| | gacgggcacagagttcatccatcacaaccagcccgagaaaggacagcgacggcgc | |
| | ggccttcagggggccggattccattaaatcttccagcagggcacaggctatctgacgc | |
| | cctttttcctcaccgtgctggcgcagataaaagccttccagctcagcggcgatggccgc | |
| | ctccagtgactcaagggtgagatgcgggtagcggtgctgacgttcgcacacggtcag | |
| | ccaggcacaggcgacagcgcgacggtaaagggcagcgcgtaagacgggggtaa | |
| | gggtgttttcatttgcttttctccctgtgacagatgactgcattccgtgccggttgcattaac | |
| | tgataaggcatatctgcgtctcctgaagacgtgcgtatccctgcgcgaatacgcacattt | |
| | aatttttcgggggtcgttttttaattacagataattgcggtaactgttatccggggtggtttc | |
| | cgggtcaggctccgtgcggggaatttcccgccattcccgcgccaccggtgctgcccg | |
| | gctgaccggaacagtgtcctgcgggtaaatatccagatatttttcccgccatttctgtaatt | |
| | ccgggtctccggccatttctttcagtaccgcatgccggtttacggggctgcgtttaaaca | |
| | ggtcaggacggtcacaggtaaattcccgcagaaaacgccccagcgggatgtctgtgg | |
| | tgcgtccgtcagcgaggatacgcacaaggatactgaatttacggcggtacgggttcca | |
| | gacaatgtccgggcagcggtacggcatttcccacggaataccgtcttccagaatgccg | |
| | accacggccacatcgggaaaaccggcagaacggtaaatctcaccgggctggggaa | |
| | aatcaaacatgcgtcctgtctccccggtctttctgctgggcgagaaaatcgcggcacag | |
| | gcctttggctttcagctcattcagcacaaaatcaatatcttcattcaggtagctgaaaatat | |
| | gtggaatgtagagctgatgcaggccggagagttcacggtgaatcaaatcacccccaa | |
| | caaaccgggatacggcgctggcgcggttgagcttatggtaagcctcaatgctgaggtg | |
| | ttcacgggcgtcatgacgcgctgagacggtctgaggggcttttttattacgcacgggac | |
| | acctccaccaccggcagacgggcagcaagggagagcacatagtcacggacaaggg | |
| | aacggcgggcactgcgttcatcaccggcgacggtgcgaagcatacagatacgggga | |
| | tgacggtctgcgcgacggacagccgcaaacacaaagacaaattcagggtgtgaggg | |
| | ggtaagggttgtagccatgatggcagcctcctgtgaatagcaaataacgctatcgccg | |
| | gagttctcacgctcgatggcgatagcccagacgggggtgagaataccggcttcacag | |
| | gataccggccagcccggaggctgccccgcctgagctaccattgactctgcggcataa | |
| | tgagcggacgcgggcaggatgcacggaatgccatctgcacgactgaccacacacca | |
| | caccataatctggcgctctgtggcattgattgcgacacaaaaaaagacgcgtggcgcg | |
| | tcatatgtcgcctgtgaattgctcgggttctcacgcccggctgccgattttgcggcaggc | |
| | gaaaaactatatccgcaaatgccggaaaaaggcaagccagaaaaagggagtttttgc | |
| | agagcgggcatcatcatgcgtcgtacccccgtttgcgtccggcaatgcgtccggccat | |
| | ccatgcggtgacttcagagtgcagccaggccacatttttaccgccaagactcacctgc | |
| | ggcggaaattcccccttacggatgagttcgtagatggtcgagcgtgacaggccgcac | |
| | aggtgcatcacttccggcacacgtaaaaaacgctcctgcgtgatgtccggcagcggc | |
| | atcagtggcgtcactggggcgggagacggggaagaaaaaacagcttgcatcgggct | |
| | acctcgttaatgtccatacagcaccggataagtccgtccggcttcgggtagcgctttattt | |
| | tgtgaatattttcagcagacgcaacaggggggatttgttcaggctgtcttacaatggctgt | |
| | gtgttttttgttcatctccacttaaagtcatttaaagccacttaaagcaatttgtaatttttatag | |
| | tgaaatacaaatcgtttcttcttattcattcccggcgaattaataaaaacaaacagtagtaa | |
| | acagcacaaaaagcccatcaacgggtgaacagtggtgaacagacggtgaacagtca | |
| | ttactgcgattgttcaccctttaacttactgtattacttatcttttttattaaggtgaacagagg | |
| | tgaacagtaaaatataaaaaaacaaacagtaagccggtttttcctgcgaccttttcctgg | |
| | cttgccggtctgaggatgagtctcctgtgtcagggctggcacatctgcaatgcgtcgtgt | |
| | tgttgtccggtgtacgtcacaattttcttaacctgaagtgacgaggagccggaaaatgtc | |
| | tgaccacactatccctgaatatctgcaacccgcactggcacaactggaaaaggccaga | |
| | gccgcccatcttgagaacgcccgcctgatggatgagaccgtcacggccattgaacgg | |
| | gcagagcaggaaaaaaatgcgctggcgcaggccgacggaaacgacgctgacgact | |
| | ggcgcacggcctttcgtgcagccggtggtgtcctgagcgacgagctgaaacagcgc | |
| | cacattgagcgcgtggcacgccgggagctggtacaggaatatgacaatctggccgtg | |
| | gtgctgaatttcgaacgtgaacgcctgaaaggggcgtgtgacagcacggccaccgcc | |
| | taccggaaggcacatcatcaccttctgagtctgtatgcagagcatgagctggaacacg | |
| | ccctgaatgaaacctgtgaggcgcttgtccgggcaatgcatctgagcattctggtacag | |
| | gaaaatccgctcgccaacaccaccggccatcagggctacgtcgcaccggaaaaggc | |
| | tgtcatgcagcaggtgaaatcatcgctggaacagaaaattaaacagatgcaaatcagc | |
| | ctcaccggcgagccggttctccggctgaccggactgtcagcggcaacactcccgcac | |
| | atggattatgaggtggcaggcacaccggcacagcgcaaggtgtggcaggacaaaat | |
| | agaccagcagggagcagagcttaaggccagagggctgctgtcatgatttactgtccgt | |
| | cgtgtggacatgttgctcacacccgtcgcgcacatttcatggacgatggcaccaagata | |
| | atgattgcacagtgccggaatatttattgctctgcgacatttgaagcgagtgaaagcttttt | |
| | ctctgacagtaaagattcaggaatggaatacatttcaggcaaacagagataccgcgatt | |
| | cactgacgtcagcctcctgcggtatgaaacgcccgaaaagaatgcttgttaccggatat | |
| | tgttgtcggagatgtaaaggccttgcactgtcaagaacatcgcggcgtctgtctcagga | |
| | agtcaccgagcgtttttatgtgtgcacggatccgggctgtggtctggtgtttaaaacgctt | |
| | cagaccatcaaccgcttcattgtccgcccggtcacgccggacgaactggcagaacgc | |
| | ctgcatgaaaaacaggaactgccgccagtacggttaaaaacacaatcatattcgctgc | |
| | gtctggaatgagggctgccggttaacaccggccgtcgccgcacaccgtatttttattctt | |
| | cagcatgatgagaaagagataacgatggaaagcacagccttacagcaggcctttgac | |
| | acctgtcagaataacaaagcagcatggctgcaacgcaaaaatgagctggcagcggc | |
| | cgaacaggaatatctgcggcttctgtcaggagaaggcagaaacgtcagtcgcctgga | |
| | cgaattacgcaatattatcgaagtcagaaaatggcaggtgaatcaggccgccggtcgtt | |
| | atattcgttcgcatgaagccgttcagcacatcagcatccgcgaccggctgaatgatttta | |
| | tgcagcagcacggcacagcactggcggccgcactggcaccggagctgatgggcta | |
| | cagtgagctgacggccattgcccgaaactgtgccatacagcgtgccacagatgccct | |
| | gcgtgaagcccttctgtcctggcttgcgaagggtgaaaaaattaattattccgcacagg | |
| | atagcgacattttaacgaccatcggattcaggcctgacgtggcttcggtggatgacagc | |
| | cgtgaaaaattcacccctgcgcagaacatgattttttcgcgtaaaagtgcgcaactggc | |
| | atcacgtcagtcagtgtaaaattccccgaaaatccgcccgtttttactgaaaaaagccat | |
| | gcatcgataaggtgcatggctttgcatgcgttttcctgcctcattttctgcaaaccgcgcc | |
| | attcccggcgcggtctgagcgtgtcagtgcaactgcattaaaaccgccccgcaaagc | |
| | gggcg | |
| pP4- | gcatgcgttttcctgcctcattttctgcaaaccgcgccattcccggcgcggtctgagcgt | pP4-c2 |
| pLtetO1- | gtcagtgcaactgcattaaaaccgccccgcaaagcgggcgggcgaggcggggaaa | |
| p72 | gcaccgcgcgcaaaccgacaagttagttaattatttgtgtagtcaaagtgccttcagtac | |
| | atacctcgttaatacattggagcataatgaagaaaatctatggcctatggtCCAAAA | |
| | CTGTCTTTTTTGATGGCACTATCCTGAAAAATATGCA | |
| | AAAAATAGATTGATGTAAGGTGGTTCTTGTCAGTGT | |
| | CGCAAGATCCTTAAGAATTCGTGGCATGAGAGAGTT | |
| | AAAGGGCGTGCGTTCCTGTATTTCCGGAGGGCCCTA | |
| | ATACCAATTCCGACGTCTAAGAAACCATTATTATCA | |
| | TGACATTAACCTATAAAAATAGGCGTATCACGAGGC | |
| | CCTTTCGTCTTCACCTCGAGTCCCTATCAGTGATAGA | |
| | GATTGACATCCCTATCAGTGATAGAGATACTGAGCA | |
| | CATCAGCAGGACGCACTGACCGAATTCGAAAGAGG | |
| | AGAAAGGAGATcatatgaaaaaggccctggcaacactgattgcactggcac | |
| | tgccggcagcagcactggcagaaggtaccgaaaacgtgcagtatcgccacgttgaac | |
| | tggcacgcgttggccaattagttgaggtggacaccctggaacacgtgcagcatattatt | |
| | ggcggtgccggcaatgacagcattacaggcaacgcccacgacaactttctggcaggc | |
| | ggcagcggcgatgatcgtctggatggtggcgccggtaatgataccctggtgggtggc | |
| | gagggtcaaaataccgttattggcggtgccggcgacgatgttttcctgcaggatctggg | |
| | cgtgtggagcaaccagctggacggtggtgcaggtgtggacaccgtgaaatacaacgt | |
| | tcatcagccgagcgaggaacgtctggaacgcatgggtgacaccggtattcatgccgat | |
| | ctgcagaaaggcaccgtggaaaaatggccggccctgaacctgtttagcgtggatcatg | |
| | ttaaaaatattgaaaatctgcatggcagccgtctgaacgaccgcattgccggcgatgac | |
| | caagataacgaactgtggggccatgacggtaatgacaccattcgcggccgtggtggc | |
| | gacgacattctgcgtggtggtctgggtctggatacactgtacggtgaggatggcaatga | |
| | tatctttctgcaggatgacgaaacagttagcgacgacattgacggtggcgcaggtctgg | |
| | acacagtggactacagcgccatgattcatccgggccgtattgtggccccgcacgaata | |
| | tggttttggtatcgaagccgatctgagccgtgaatgggtgcgcaaagcaagtgccctg | |
| | ggtgtggactattacgacaacgtgcgcaatgtggaaaacgtgattggcaccagcatga | |
| | aagacgtgctgattggcgacgcccaggccaacaccctgatgggccagggtggtgac | |
| | gatacagtgcgcggtggtgatggtgatgacctgctgttcggcggcgatggtaacgaca | |
| | tgctgtatggcgatgccggtaacgacaccctgtacggcggtctgggtgacgataccct | |
| | ggagggcggtgccggcaacgattggtttggccaaacccaggcccgtgaacatgatgt | |
| | gctgcgcggtggcgatggcgtggataccgttgactacagtcagacaggcgcccatgc | |
| | aggtatcgcagccggtcgcattggtctgggtattctggcagacctgggtgccggtcgt | |
| | gtggataaactgggtgaggccggcagtagtgcctatgacacagtgagcggcatcgaa | |
| | aacgtggtgggcacagagctggcagaccgtatcacaggcgatgcccaagccaatgtt | |
| | ctgcgcggcgcaggtggtgccgatgtgttagccggtggtgagggtgatgacgttctgc | |
| | tgggcggtgacggcgatgatcagctgagtggtgatgcaggtcgcgaccgcctgtatg | |
| | gcgaagcaggcgatgattggttctttcaggacgcagccaatgccggtaatctgttagac | |
| | ggtggcgacggtcgtgatacagtggattttagcggtcctggtcgtggcctggatgccg | |
| | gcgcaaagggtgtgtttctgagcctgggcaaaggctttgccagcttaatggacgaacc | |
| | ggaaaccagcaacgtgttacgcaacattgagaacgccgtgggtagtgcccgcgatga | |
| | cgtgctgatcggtgatgccggtgcaaatgttctgaacggcctggccggcaacgacgtg | |
| | ctgagtggtggtgcaggtgatgatgttctgctgggtgatgagggcagtgacttactgag | |
| | cggtgacgccggcaatgacgatttattcggcggccagggtgatgacacctacctgtttg | |
| | gcgtgggttatggccacgacaccatttatgagagcggtggtggtcacgacacaatccg | |
| | catcaatgcaggcgccgatcagctgtggtttgcccgccagggtaacgatctggagatt | |
| | cgtatcctgggcacagacgatgccttaacagtgcatgattggtaccgcgatgccgatca | |
| | tcgcgtggagattatccatgcagcaaaccaggcagtggatcaggccggcatcgagaa | |
| | gctggttgaggccatggcccagtatcctgatcctggtggtcatcatcaccaccatcatta | |
| | ataactcgagtaaCCCGGGCTCGGTACCAAATTCCAGAAAA | |
| | GAGGCCTCCCGAAAGGGGGGCCTTTTTTCGTTTTGGT | |
| | CCGAGCTCTGGTAAATAGAAACGGAACTTTACATAT | |
| | TGAATACCGGAGATATAATTCGTTTATGCCACATAA | |
| | TCATCAATACATCCGTAACCCGCCCGAAACGGAAAA | |
| | ACTGAAGAAAAACCCCATTATCTTAGCCTAActagcataa | |
| | ccccttggggcctctaaacgggtcttgaggggttttttgagacaaacaaaagaatggaa | |
| | tcaaagttaaTGCTAGCAGCcGCCGctgcaggcatgcaagcttgcggcc | |
| | gcgtcgtgactgggaaaaccctggcgactagtcttggactcctgttgatagatccagta | |
| | atgacctcagaactccatctggatttgttcagaacgctcggttgccgccgggcgtttttta | |
| | ttggtgagaatccaggggtccccaataattacgatttaaatttgtgtctcaaaatctctgat | |
| | gttacattgcacaagataaaaatatatcatcatgaacaataaaactgtctgcttacataaa | |
| | cagtaatacaaggggtgttatgagccatattcagcgtgaaacgagctgtagccgtccg | |
| | cgtctgaacagcaacatggatgcggatctgtatggctataaatgggcgcgtgataacgt | |
| | gggtcagagcggcgcgaccatttatcgtctgtatggcaaaccggatgcgccggaact | |
| | gtttctgaaacatggcaaaggcagcgtggcgaacgatgtgaccgatgaaatggtgcgt | |
| | ctgaactggctgaccgaatttatgccgctgccgaccattaaacattttattcgcaccccg | |
| | gatgatgcgtggctgctgaccaccgcgattccgggcaaaaccgcgtttcaggtgctgg | |
| | aagaatatccggatagcggcgaaaacattgtggatgcgctggccgtgtttctgcgtcgt | |
| | ctgcatagcattccggtgtgcaactgcccgtttaacagcgatcgtgtgtttcgtctggcc | |
| | caggcgcagagccgtatgaacaacggcctggtggatgcgagcgattttgatgatgaa | |
| | cgtaacggctggccggtggaacaggtgtggaaagaaatgcataaactgctgccgttta | |
| | gcccggatagcgtggtgacccacggcgattttagcctggataacctgattttcgatgaa | |
| | ggcaaactgattggctgcattgatgtgggccgtgtgggcattgcggatcgttatcagga | |
| | tctggccattctgtggaactgcctgggcgaatttagcccgagcctgcaaaaacgtctgtt | |
| | tcagaaatatggcattgataatccggatatgaacaaactgcaatttcatctgatgctggat | |
| | gaatttttctaacttggaagtaagaatggtgccgaaggccggactcaaacatcaaaata | |
| | agttaatgataaaaaacaaataataaaacacaacaatgaaatatgcccccttttgtgccc | |
| | ccactgtttttctgaccaatctattttcagcccatcaataaatcggaaagttaaatcattttta | |
| | atcagtaagtttggatccgtagctcggatccaaaccagtgcatcttttatccacataaaaa | |
| | attttttttcgaaagaactgttcacactgttcacctttctgttttctccttttatttcagagtgata | |
| | ggtggtgaataatgggtgaagggtgaacattcgattcttcacctccggcattctgccgat | |
| | gtgactcataccggtgattaatcctccgcactgaaatcactcaggaagaaaaaagtttttt | |
| | ttgatttgattgttcacactgttcacctttcgtttttctcttttaatttcagtgtgataacgggtg | |
| | aatatacggtgaagggtgaacagtggattgttcaccttcgggggatatcgggataaaaa | |
| | aagaccggcagatgccggtcaggtgggtcaggctgttgtagggtcgtcacattttggc | |
| | agccagtcgccgtagctttcctctttcagcgtcaggttggtctgtatcccctgtttggtatg | |
| | gcgtttctcgtaattcagtccgtattccttcagcatcaccggcagccccagcccgaacat | |
| | tttcagactgagtacattccggtagccgtttgcctccatgtaggccagataggcgtgata | |
| | gaggtatttacggtaattgcgcgggatgatactggcgttccccatatacatgccgctggt | |
| | ctgcggcagggtttccagatagccgataaaatcaaacgtcgggtcggcatcccgtttg | |
| | atgttcagtgcctcgtctgagttctgctgggactgaagcagtgaccgggcgagcatcg | |
| | ggtcgctgaacttctgcatcaggtgacgcacgatgaccgccagctcgcgggtgattttg | |
| | tccttaagctgcgggtcgcgctcctgcggggctatctgttccgggaagtgaataatcac | |
| | ccgtcggcgtgacacgccgccgctgcggtcggtgaagcgcatcgggttattgttcacg | |
| | gccagaatcaccgccgggatgtgcgtggagtacgcatcccggtatttcgggtcaacg | |
| | gacaccgcatcgccgccggtgatggccttgagtccggcaccgtcgccgctccatttttc | |
| | ctggtccggcaggcgtatcagtgagaagccagttaacgcggcacgttcacgcgggg | |
| | attccagcgtctcgatggtggccgacgtggcgttatcctccccggccagcagggtggc | |
| | tatttcggccatgatacttttgccgctgccgccgggaccggtcacctccagaaagagct | |
| | gccagtcgtagcggtttgccagcaccataaacagtgcagccagaatcacgtcgcgtttt | |
| | tccgcacggccaccggcggcacggtcaagccagcgccagaaggcgggggcgtgg | |
| | gtttccagcgtttcaccgtccaccggggggtgaaatccacatcgcacagggtgcgca | |
| | tccagtgtgacggactgtgcgggtggaacgtgccgttctgcgtgtcgagcacgccgtt | |
| | acgaaagccaatcaggcggcgggagggggcttcctgctgcggaataatcagcttcag | |
| | ggtatccaccacggaggccaccttcccggaggagaacggcgcacgcagacgctga | |
| | aacagcccggccacatcccgggcaaagtcctgtggcggcagcaccttccagacacc | |
| | attttcatagcgggacagaagctggccgttggcatcgaccgcgagcgcctcgccgtaa | |
| | tgctcatagatacgcatggccttttcgctggtactcatggcggaaaactccgcttcgctc | |
| | atggtgtcgaacgggctttcagccggtggccggatggcatcgtaaatggccttacggg | |
| | tggcctccccgccgtactgcgtgaaggcatcattccagtcaccgaagaccggcggca | |
| | gggcaacaacaccttcacacgcatctgcggctgcggcggcttttttctggccgtcacca | |
| | ctgaggtcacggtcagcggcaaggacaatctgacaggcggggtgcttctgccgggc | |
| | aaggctggccagagaaaggaggttcacggaagaaagcgccaccatcaccgtttcac | |
| | cggtcaggtgatgtacggtaagtgcggtcgcgtatccctccgctatccacagacgttttc | |
| | cggcctgattctgtccttcaagggtgtgacaggtgcccctgacctgtccgcctttcagg | |
| | gtgcgcttacggccgtcagcactgattaactgaaggttaaccagttcgccgctgtcgtc | |
| | atacagtggcaccacaaggtcaccggcgcgccagctcacgccaccggctctgtgtgt | |
| | gccggtcagcatccggcattcccggccgggaaagcccttgcgggtcaggtaggcgtt | |
| | accggttccggtacgggttttcgccatcagggtttgtgccagtgcggcggcgttcttcc | |
| | gggcagcgtctgtttcatcaacggcggcggtcgtcactgccgggtcagccggtggca | |
| | ggctgccggtcacggcagccacctttgcggccgcgtcggacggggaaacaccaaaa | |
| | accttttcaaccagtttcaggccgtcaccggcaccacactgattgcagtaccaggtgcc | |
| | gcgcccctccctgtcatcaaaacggaagcggtcactcccgccacagaccggacagg | |
| | gctgatgacggttcttcagcacctgaatccccagcgccgggagaatacgcggccagt | |
| | ggccgagcgcatggctgacggtggcggttacgttcattttcatggtgttgttctccttcag | |
| | tgcagtaccggcgcttttatgtgacgggcacagagttcatccatcacaaccagcccga | |
| | gaaaggacagcgacggcgcggccttcagggggccggattccattaaatcttccagca | |
| | gggcacaggctatctgacgccctttttcctcaccgtgctggcgcagataaaagccttcc | |
| | agctcagcggcgatggccgcctccagtgactcaagggtgagatgcgggtagcggtg | |
| | ctgacgttcgcacacggtcagccaggcacaggcgacagcgcgacggtaaagggca | |
| | gcgcgtaagacgggcggtaagggtgttttcatttgcttttctccctgtgacagatgactg | |
| | cattccgtgccggttgcattaactgataaggcatatctgcgtctcctgaagacgtgcgtat | |
| | ccctgcgcgaatacgcacatttaatttttcgggggtcgttttttaattacagataattgcggt | |
| | aactgttatccggggtggtttccgggtcaggctccgtgcggggaatttcccgccattcc | |
| | cgcgccaccggtgctgcccggctgaccggaacagtgtcctgcgggtaaatatccaga | |
| | tatttttcccgccatttctgtaattccgggtctccggccatttctttcagtaccgcatgccgg | |
| | tttacggggctgcgtttaaacaggtcaggacggtcacaggtaaattcccgcagaaaac | |
| | gccccagcgggatgtctgtggtgcgtccgtcagcgaggatacgcacaaggatactga | |
| | atttacggcggtacgggttccagacaatgtccgggcagcggtacggcatttcccacgg | |
| | aataccgtcttccagaatgccgaccacggccacatcgggaaaaccggcagaacggta | |
| | aatctcaccgggctggggaaaatcaaacatgcgtcctgtctccccggtctttctgctgg | |
| | gcgagaaaatcgcggcacaggcctttggctttcagctcattcagcacaaaatcaatatc | |
| | ttcattcaggtagctgaaaatatgtggaatgtagagctgatgcaggccggagagttcac | |
| | ggtgaatcaaatcacccccaacaaaccgggatacggcgctggcgcggttgagcttat | |
| | ggtaagcctcaatgctgaggtgttcacgggcgtcatgacgcgctgagacggtctgag | |
| | gggcttttttattacgcacgggacacctccaccaccggcagacgggcagcaagggag | |
| | agcacatagtcacggacaagggaacgggggcactgcgttcatcaccggcgacggt | |
| | gcgaagcatacagatacggggatgacggtctgcgcgacggacagccgcaaacaca | |
| | aagacaaattcagggtgtgagggggtaagggttgtagccatgatggcagcctcctgtg | |
| | aatagcaaataacgctatcgccggagttctcacgctcgatggcgatagcccagacgg | |
| | gggtgagaataccggcttcacaggataccggccagcccggaggctgccccgcctga | |
| | gctaccattgactctgcggcataatgagcggacgcgggcaggatgcacggaatgcca | |
| | tctgcacgactgaccacacaccacaccataatctggcgctctgtggcattgattgcgac | |
| | acaaaaaaagacgcgtggcgcgtcatatgtcgcctgtgaattgctcgggttctcacgcc | |
| | cggctgccgattttgcggcaggcgaaaaactatatccgcaaatgccggaaaaaggca | |
| | agccagaaaaagggagtttttgcagagcgggcatcatcatgcgtcgtacccccgtttg | |
| | cgtccggcaatgcgtccggccatccatgcggtgacttcagagtgcagccaggccaca | |
| | tttttaccgccaagactcacctgcggcggaaattcccccttacggatgagttcgtagatg | |
| | gtcgagcgtgacaggccgcacaggtgcatcacttccggcacacgtaaaaaacgctcc | |
| | tgcgtgatgtccggcagcggcatcagtggcgtcactggggcgggagacggggaag | |
| | aaaaaacagcttgcatcgggctacctcgttaatgtccatacagcaccggataagtccgt | |
| | ccggcttcgggtagcgctttattttgtgaatattttcagcagacgcaacaggggggattt | |
| | gttcaggctgtcttacaatggctgtgtgttttttgttcatctccacttaaagtcatttaaagcc | |
| | acttaaagcaatttgtaatttttatagtgaaatacaaatcgtttcttcttattcattcccggcg | |
| | aattaataaaaacaaacagtagtaaacagcacaaaaagcccatcaacgggtgaacagt | |
| | ggtgaacagacggtgaacagtcattactgcgattgttcaccctttaacttactgtattactt | |
| | atcttttttattaaggtgaacagaggtgaacagtaaaatataaaaaaacaaacagtaagc | |
| | cggtttttcctgcgaccttttcctggcttgccggtctgaggatgagtctcctgtgtcaggg | |
| | ctggcacatctgcaatgcgtcgtgttgttgtccggtgtacgtcacaattttcttaacctgaa | |
| | gtgacgaggagccggaaaatgtctgaccacactatccctgaatatctgcaacccgcac | |
| | tggcacaactggaaaaggccagagccgcccatcttgagaacgcccgcctgatggatg | |
| | agaccgtcacggccattgaacgggcagagcaggaaaaaaatgcgctggcgcaggc | |
| | cgacggaaacgacgctgacgactggcgcacggcctttcgtgcagccggtggtgtcct | |
| | gagcgacgagctgaaacagcgccacattgagcgcgtggcacgccgggagctggta | |
| | caggaatatgacaatctggccgtggtgctgaatttcgaacgtgaacgcctgaaagggg | |
| | cgtgtgacagcacggccaccgcctaccggaaggcacatcatcaccttctgagtctgta | |
| | tgcagagcatgagctggaacacgccctgaatgaaacctgtgaggcgcttgtccgggc | |
| | aatgcatctgagcattctggtacaggaaaatccgctcgccaacaccaccggccatcag | |
| | ggctacgtcgcaccggaaaaggctgtcatgcagcaggtgaaatcatcgctggaacag | |
| | aaaattaaacagatgcaaatcagcctcaccggcgagccggttctccggctgaccgga | |
| | ctgtcagcggcaacactcccgcacatggattatgaggtggcaggcacaccggcacag | |
| | cgcaaggtgtggcaggacaaaatagaccagcagggagcagagcttaaggccagag | |
| | ggctgctgtcatgatttactgtccgtcgtgtggacatgttgctcacacccgtcgcgcaca | |
| | tttcatggacgatggcaccaagataatgattgcacagtgccggaatatttattgctctgcg | |
| | acatttgaagcgagtgaaagctttttctctgacagtaaagattcaggaatggaatacattt | |
| | caggcaaacagagataccgcgattcactgacgtcagcctcctgcggtatgaaacgcc | |
| | cgaaaagaatgcttgttaccggatattgttgtcggagatgtaaaggccttgcactgtcaa | |
| | gaacatcgcggcgtctgtctcaggaagtcaccgagcgtttttatgtgtgcacggatccg | |
| | ggctgtggtctggtgtttaaaacgcttcagaccatcaaccgcttcattgtccgcccggtc | |
| | acgccggacgaactggcagaacgcctgcatgaaaaacaggaactgccgccagtacg | |
| | gttaaaaacacaatcatattcgctgcgtctggaatgagggctgccggttaacaccggcc | |
| | gtcgccgcacaccgtatttttattcttcagcatgatgagaaagagataacgatggaaagc | |
| | acagccttacagcaggcctttgacacctgtcagaataacaaagcagcatggctgcaac | |
| | gcaaaaatgagctggcagcggccgaacaggaatatctgcggcttctgtcaggagaag | |
| | gcagaaacgtcagtcgcctggacgaattacgcaatattatcgaagtcagaaaatggca | |
| | ggtgaatcaggccgccggtcgttatattcgttcgcatgaagccgttcagcacatcagca | |
| | tccgcgaccggctgaatgattttatgcagcagcacggcacagcactggcggccgcac | |
| | tggcaccggagctgatgggctacagtgagctgacggccattgcccgaaactgtgcca | |
| | tacagcgtgccacagatgccctgcgtgaagcccttctgtcctggcttgcgaagggtga | |
| | aaaaattaattattccgcacaggatagcgacattttaacgaccatcggattcaggcctga | |
| | cgtggcttcggtggatgacagccgtgaaaaattcacccctgcgcagaacatgattttttc | |
| | gcgtaaaagtgcgcaactggcatcacgtcagtcagtgtaaaattccccgaaaatccgc | |
| | ccgtttttactgaaaaaagccatgcatcgataaggtgcatggcttt | |
| pP4- | gcatgcgttttcctgcctcattttctgcaaaccgcgccattcccggcgcggtctgagcgt | pP4-c2 |
| LacIq- | gtcagtgcaactgcattaaaaccgccccgcaaagcgggcgggcgaggcggggaaa | |
| p65 | gcaccgcgcgcaaaccgacaagttagttaattatttgtgtagtcaaagtgccttcagtac | |
| | atacctcgttaatacattggagcataatgaagaaaatctatggcctatggtCCAAAA | |
| | CTGTCTTTTTTGATGGCACTATCCTGAAAAATATGCA | |
| | AAAAATAGATTGATGTAAGGTGGTTCTTGTCAGTGT | |
| | CGCAAGATCCTTAAGAATTCGTGGCATGAGAGAGTT | |
| | AAAGGGCGTGCGTTCCTGTATTTCCGGAGGGCCCTA | |
| | ATACTCGAATGGTGCAAAACCTTTCGCGGTATGGCA | |
| | TGATAGCGCCGAATTCGAAAGAGGAGAAAGGAGATc | |
| | atatgctctccacactccgccgcactctatttgcgctgctggcttgtgcgtcttttatcgtc | |
| | catgccgatccgccggccaccgtgtataaatatgatagccgcccgccggaagatgtgt | |
| | ttcagaatggcttcaccgcctggggcaacaatgataacgtgctggaccacctgaccgg | |
| | tcgtagcggtcaggtgggtagcagcaacagtgcctttgtgagcaccagcagcagccg | |
| | tcgctataccgaagtgtacctggaacatcgcatgcaggaagcagtggaagcagaacg | |
| | tgcaggccgtggcaccggccattttattggctacatttatgaggtgcgcgccgataataa | |
| | cttttacggcgccgccagcagctactttgaatacgtggacacctacggcgacaatgcc | |
| | ggtcgtatcctggcaggtgccctggcaacctatcagagcggttatctggcccatcgcc | |
| | gcattccgccggaaaatattcgccgtgtgacccgcgtgtatcataacggcattaccggc | |
| | gaaaccaccaccaccgagtatagcaatgcccgctatgttagccagcagacccgcgcc | |
| | aatccgaatccgtataccagcggcggtcatcatcaccaccaccattaataactcCTT | |
| | TAGCGTGGATAAAAAATAAtaaCCCGGGCTCGGTACC | |
| | AAATTCCAGAAAAGAGGCCTCCCGAAAGGGGGGCC | |
| | TTTTTTCGTTTTGGTCCTGGTAAATAGAAACGGAACT | |
| | TTACATATTGAATACCGGAGATATAATTCGTTTATGC | |
| | CACATAATCATCAATACATCCGTAACCCGCCCGAAA | |
| | CGGAAAAACTGAAGAAAAACCCCATTATCTTAGCCT | |
| | AAAAAAACCTTCTCTTTGGAACTTTCAGTAATACGCT | |
| | TAACTGCTCATTGCTATATTGAAGTACGGATTAGAA | |
| | GCCGCCGAGCGGGTGACAGCCCTCCGAAGGAAGAC | |
| | TCTCCTCCGTGCGTCCTCGTCTTCACCGGTCGCGTTC | |
| | CTGAAACGCAGATGTGCCTCGCGCCGCACTGCTCCG | |
| | AACAATAAAGATTCTACAATACTAGCTTTTATGGTT | |
| | ATGAAGAGGAAAAATTGGCAGTAACCTGGCCCCAC | |
| | AAACCTTCAAATGAACGAATCAAATTAACAACCATA | |
| | GGATGATAATGCGATTAGTTTTTTAGCCTTATTTCTG | |
| | GGGTAATTAATCAGCGAAGCGATGATTTTTGATCTA | |
| | TTAACAGATATATAAATGCAAAAACTGCATAACCAC | |
| | TTTAACTAATACTTTCAACATTTTCGGTTTGTATTAC | |
| | TTCTTATTCAAATGTAATAAAAGTATCAACAAAAAA | |
| | TTGTTAATATACCTCTATACTTTAACGTCAAGGAGGA | |
| | AACTAGACCCGCCGCCACCATGGAGATGAGTGACGC | |
| | GGCACCGAGCTTGTCTAACCTTTTCTATGATCCGACC | |
| | TACAACCCTGGGCAAAGTACGATTGATTACAAAGAC | |
| | GATGACGACAAGGCCGAGATCTATAATAAAGACGG | |
| | AAATAAATTGGATTTGTTCGGTAAAGTCGACGGACT | |
| | TCACTACTTCTCTGATGACAAGGGAAGTGACGGAGA | |
| | CCAGACTTACATGAGAATCGGATTCAAGGGTGAGAC | |
| | CCAAGTTAACGACCAGCTTACCGGATACGGTCAGTG | |
| | GGAGTACCAGATCCAAGGAAACCAGACCGAGGGTA | |
| | GTAACGACTCTTGGACCAGAGTCGCTTTCGCCGGTTT | |
| | GAAGTTCGCTGACGCTGGTAGTTTCGACTACGGAAG | |
| | GAATTACGGAGTCACCTACGACGTTACCTCTTGGAC | |
| | CGACGTCCTTCCAGAATTCGGTGGAGATACCTACGG | |
| | TGCCGACAACTTCATGCAGCAAAGGGGTAACGGTTA | |
| | TGCCACCTATAGAAACACCGACTTCTTCGGTTTGGTC | |
| | GACGGTTTGGACTTCGCCTTGCAGTACCAAGGTAAG | |
| | AATGGTAGTGTTTCTGGTGAAAATACCAATGGTAGA | |
| | TCTTTGCTTAATCAGAACGGAGACGGTTACGGTGGT | |
| | TCTTTGACCTACGCCATCGGTGAAGGTTTCTCTGTCG | |
| | GAGGTGCCATTACCACCAGTAAGAGAACCGCCGACC | |
| | AGAACAATACCGCCAACGCCAGATTGTACGGAAAC | |
| | GGTGATAGAGCCACCGTCTACACCGGTGGTTTGAAA | |
| | TACGACGCTAACAACATCTACCTTGCTGCCCAGTAC | |
| | AGTCAGACCTACAATGCCACTAGATTCGGAACCAGT | |
| | AACGGTAGTAACCCAAGTACCTCTTACGGATTTGCC | |
| | AACAAGGCCCAGAACTTTGAGGTCGTTGCCCAGTAC | |
| | CAGTTCGACTTCGGTCTTAGGCCAAGTGTCGCTTATC | |
| | TTCAGTCTAAGGGTAAGGACATCTCTAACGGTTACG | |
| | GAGCCTCTTACGGTGACCAAGATATCGTCAAGTACG | |
| | TCGACGTCGGAGCTACCTACTACTTTAATAAAAACA | |
| | TGAGTACTTACGTCGATTATAAGATCAATTTGTTGGA | |
| | CAAAAACGACTTCACTAGAGATGCCGGTATCAATcta | |
| | gcataaccccttggggcctctaaacgggtcttgaggggttttttgagacaaacaaaaga | |
| | atggaatcaaagttaaTGCTAGCAGCcGCCGctgcaggcatgcaagcttg | |
| | cggccgcgtcgtgactgggaaaaccctggcgactagtcttggactcctgttgatagatc | |
| | cagtaatgacctcagaactccatctggatttgttcagaacgctcggttgccgccgggcg | |
| | ttttttattggtgagaatccaggggtccccaataattacgatttaaatttgtgtctcaaaatct | |
| | ctgatgttacattgcacaagataaaaatatatcatcatgaacaataaaactgtctgcttaca | |
| | taaacagtaatacaaggggtgttatgagccatattcagcgtgaaacgagctgtagccgt | |
| | ccgcgtctgaacagcaacatggatgcggatctgtatggctataaatgggcgcgtgata | |
| | acgtgggtcagagcggcgcgaccatttatcgtctgtatggcaaaccggatgcgccgg | |
| | aactgtttctgaaacatggcaaaggcagcgtggcgaacgatgtgaccgatgaaatggt | |
| | gcgtctgaactggctgaccgaatttatgccgctgccgaccattaaacattttattcgcacc | |
| | ccggatgatgcgtggctgctgaccaccgcgattccgggcaaaaccgcgtttcaggtg | |
| | ctggaagaatatccggatagcggcgaaaacattgtggatgcgctggccgtgtttctgc | |
| | gtcgtctgcatagcattccggtgtgcaactgcccgtttaacagcgatcgtgtgtttcgtct | |
| | ggcccaggcgcagagccgtatgaacaacggcctggtggatgcgagcgattttgatga | |
| | tgaacgtaacggctggccggtggaacaggtgtggaaagaaatgcataaactgctgcc | |
| | gtttagcccggatagcgtggtgacccacggcgattttagcctggataacctgattttcga | |
| | tgaaggcaaactgattggctgcattgatgtgggccgtgtgggcattgcggatcgttatc | |
| | aggatctggccattctgtggaactgcctgggcgaatttagcccgagcctgcaaaaacg | |
| | tctgtttcagaaatatggcattgataatccggatatgaacaaactgcaatttcatctgatgc | |
| | tggatgaatttttctaacttggaagtaagaatggtgccgaaggccggactcaaacatca | |
| | aaataagttaatgataaaaaacaaataataaaacacaacaatgaaatatgcccccttttgt | |
| | gcccccactgtttttctgaccaatctattttcagcccatcaataaatcggaaagttaaatca | |
| | tttttaatcagtaagtttggatccgtagctcggatccaaaccagtgcatcttttatccacata | |
| | aaaaattttttttcgaaagaactgttcacactgttcacctttctgttttctccttttatttcagag | |
| | tgataggtggtgaataatgggtgaagggtgaacattcgattcttcacctccggcattctg | |
| | ccgatgtgactcataccggtgattaatcctccgcactgaaatcactcaggaagaaaaaa | |
| | gttttttttgatttgattgttcacactgttcacctttcgtttttctcttttaatttcagtgtgataac | |
| | gggtgaatatacggtgaagggtgaacagtggattgttcaccttcgggggatatcggga | |
| | taaaaaaagaccggcagatgccggtcaggtgggtcaggctgttgtagggtcgtcacat | |
| | tttggcagccagtcgccgtagctttcctctttcagcgtcaggttggtctgtatcccctgttt | |
| | ggtatggcgtttctcgtaattcagtccgtattccttcagcatcaccggcagccccagccc | |
| | gaacattttcagactgagtacattccggtagccgtttgcctccatgtaggccagataggc | |
| | gtgatagaggtatttacggtaattgcgcgggatgatactggcgttccccatatacatgcc | |
| | gctggtctgcggcagggtttccagatagccgataaaatcaaacgtcgggtcggcatcc | |
| | cgtttgatgttcagtgcctcgtctgagttctgctgggactgaagcagtgaccgggcgag | |
| | catcgggtcgctgaacttctgcatcaggtgacgcacgatgaccgccagctcgcgggt | |
| | gattttgtccttaagctgcgggtcgcgctcctgcggggctatctgttccgggaagtgaat | |
| | aatcacccgtcggcgtgacacgccgccgctgcggtcggtgaagcgcatcgggttatt | |
| | gttcacggccagaatcaccgccgggatgtgcgtggagtacgcatcccggtatttcggg | |
| | tcaacggacaccgcatcgccgccggtgatggccttgagtccggcaccgtcgccgctc | |
| | catttttcctggtccggcaggcgtatcagtgagaagccagttaacgcggcacgttcacg | |
| | cggggattccagcgtctcgatggtggccgacgtggcgttatcctccccggccagcag | |
| | ggtggctatttcggccatgatacttttgccgctgccgccgggaccggtcacctccagaa | |
| | agagctgccagtcgtagcggtttgccagcaccataaacagtgcagccagaatcacgtc | |
| | gcgtttttccgcacggccaccggcggcacggtcaagccagcgccagaaggcgggg | |
| | gcgtgggtttccagcgtttcaccgtccaccggcggggtgaaatccacatcgcacagg | |
| | gtgcgcatccagtgtgacggactgtgcgggtggaacgtgccgttctgcgtgtcgagca | |
| | cgccgttacgaaagccaatcaggcggcgggagggggcttcctgctgcggaataatca | |
| | gcttcagggtatccaccacggaggccaccttcccggaggagaacggcgcacgcaga | |
| | cgctgaaacagcccggccacatcccgggcaaagtcctgtggcggcagcaccttcca | |
| | gacaccattttcatagcgggacagaagctggccgttggcatcgaccgcgagcgcctc | |
| | gccgtaatgctcatagatacgcatggccttttcgctggtactcatggcggaaaactccg | |
| | cttcgctcatggtgtcgaacgggctttcagccggtggccggatggcatcgtaaatggc | |
| | cttacgggtggcctccccgccgtactgcgtgaaggcatcattccagtcaccgaagacc | |
| | ggcggcagggcaacaacaccttcacacgcatctgcggctgcggcggcttttttctggc | |
| | cgtcaccactgaggtcacggtcagcggcaaggacaatctgacaggcggggtgcttct | |
| | gccgggcaaggctggccagagaaaggaggttcacggaagaaagcgccaccatcac | |
| | cgtttcaccggtcaggtgatgtacggtaagtgcggtcgcgtatccctccgctatccaca | |
| | gacgttttccggcctgattctgtccttcaagggtgtgacaggtgcccctgacctgtccgc | |
| | ctttcagggtgcgcttacggccgtcagcactgattaactgaaggttaaccagttcgccg | |
| | ctgtcgtcatacagtggcaccacaaggtcaccggcgcgccagctcacgccaccggct | |
| | ctgtgtgtgccggtcagcatccggcattcccggccgggaaagcccttgcgggtcaggt | |
| | aggcgttaccggttccggtacgggttttcgccatcagggtttgtgccagtgcggcggc | |
| | gttcttccgggcagcgtctgtttcatcaacggcggcggtcgtcactgccgggtcagcc | |
| | ggtggcaggctgccggtcacggcagccacctttgcggccgcgtcggacggggaaa | |
| | caccaaaaaccttttcaaccagtttcaggccgtcaccggcaccacactgattgcagtac | |
| | caggtgccgcgcccctccctgtcatcaaaacggaagcggtcactcccgccacagacc | |
| | ggacagggctgatgacggttcttcagcacctgaatccccagcgccgggagaatacgc | |
| | ggccagtggccgagcgcatggctgacggtggcggttacgttcattttcatggtgttgttc | |
| | tccttcagtgcagtaccggcgcttttatgtgacgggcacagagttcatccatcacaacca | |
| | gcccgagaaaggacagcgacggcgcggccttcagggggccggattccattaaatctt | |
| | ccagcagggcacaggctatctgacgccctttttcctcaccgtgctggcgcagataaaa | |
| | gccttccagctcagcggcgatggccgcctccagtgactcaagggtgagatgcgggta | |
| | gcggtgctgacgttcgcacacggtcagccaggcacaggcgacagcgcgacggtaa | |
| | agggcagcgcgtaagacgggcggtaagggtgttttcatttgcttttctccctgtgacaga | |
| | tgactgcattccgtgccggttgcattaactgataaggcatatctgcgtctcctgaagacgt | |
| | gcgtatccctgcgcgaatacgcacatttaatttttcgggggtcgttttttaattacagataat | |
| | tgcggtaactgttatccggggtggtttccgggtcaggctccgtgcggggaatttcccgc | |
| | cattcccgcgccaccggtgctgcccggctgaccggaacagtgtcctgcgggtaaatat | |
| | ccagatatttttcccgccatttctgtaattccgggtctccggccatttctttcagtaccgcat | |
| | gccggtttacggggctgcgtttaaacaggtcaggacggtcacaggtaaattcccgcag | |
| | aaaacgccccagcgggatgtctgtggtgcgtccgtcagcgaggatacgcacaaggat | |
| | actgaatttacggcggtacgggttccagacaatgtccgggcagcggtacggcatttcc | |
| | cacggaataccgtcttccagaatgccgaccacggccacatcgggaaaaccggcaga | |
| | acggtaaatctcaccgggctggggaaaatcaaacatgcgtcctgtctccccggtctttct | |
| | gctgggcgagaaaatcgcggcacaggcctttggctttcagctcattcagcacaaaatc | |
| | aatatcttcattcaggtagctgaaaatatgtggaatgtagagctgatgcaggccggaga | |
| | gttcacggtgaatcaaatcacccccaacaaaccgggatacggcgctggcgcggttga | |
| | gcttatggtaagcctcaatgctgaggtgttcacgggcgtcatgacgcgctgagacggt | |
| | ctgaggggcttttttattacgcacgggacacctccaccaccggcagacgggcagcaa | |
| | gggagagcacatagtcacggacaagggaacggcgggcactgcgttcatcaccggc | |
| | gacggtgcgaagcatacagatacggggatgacggtctgcgcgacggacagccgca | |
| | aacacaaagacaaattcagggtgtgagggggtaagggttgtagccatgatggcagcc | |
| | tcctgtgaatagcaaataacgctatcgccggagttctcacgctcgatggcgatagccca | |
| | gacgggggtgagaataccggcttcacaggataccggccagcccggaggctgcccc | |
| | gcctgagctaccattgactctgcggcataatgagcggacgcgggcaggatgcacgga | |
| | atgccatctgcacgactgaccacacaccacaccataatctggcgctctgtggcattgatt | |
| | gcgacacaaaaaaagacgcgtggcgcgtcatatgtcgcctgtgaattgctcgggttct | |
| | cacgcccggctgccgattttgcggcaggcgaaaaactatatccgcaaatgccggaaa | |
| | aaggcaagccagaaaaagggagtttttgcagagcgggcatcatcatgcgtcgtaccc | |
| | ccgtttgcgtccggcaatgcgtccggccatccatgcggtgacttcagagtgcagccag | |
| | gccacatttttaccgccaagactcacctgcggcggaaattcccccttacggatgagttc | |
| | gtagatggtcgagcgtgacaggccgcacaggtgcatcacttccggcacacgtaaaaa | |
| | acgctcctgcgtgatgtccggcagcggcatcagtggcgtcactggggcgggagacg | |
| | gggaagaaaaaacagcttgcatcgggctacctcgttaatgtccatacagcaccggata | |
| | agtccgtccggcttcgggtagcgctttattttgtgaatattttcagcagacgcaacaggg | |
| | gggatttgttcaggctgtcttacaatggctgtgtgttttttgttcatctccacttaaagtcattt | |
| | aaagccacttaaagcaatttgtaatttttatagtgaaatacaaatcgtttcttcttattcattcc | |
| | cggcgaattaataaaaacaaacagtagtaaacagcacaaaaagcccatcaacgggtg | |
| | aacagtggtgaacagacggtgaacagtcattactgcgattgttcaccctttaacttactgt | |
| | attacttatcttttttattaaggtgaacagaggtgaacagtaaaatataaaaaaacaaaca | |
| | gtaagccggtttttcctgcgaccttttcctggcttgccggtctgaggatgagtctcctgtgt | |
| | cagggctggcacatctgcaatgcgtcgtgttgttgtccggtgtacgtcacaattttcttaa | |
| | cctgaagtgacgaggagccggaaaatgtctgaccacactatccctgaatatctgcaac | |
| | ccgcactggcacaactggaaaaggccagagccgcccatcttgagaacgcccgcctg | |
| | atggatgagaccgtcacggccattgaacgggcagagcaggaaaaaaatgcgctggc | |
| | gcaggccgacggaaacgacgctgacgactggcgcacggcctttcgtgcagccggtg | |
| | gtgtcctgagcgacgagctgaaacagcgccacattgagcgcgtggcacgccgggag | |
| | ctggtacaggaatatgacaatctggccgtggtgctgaatttcgaacgtgaacgcctgaa | |
| | aggggcgtgtgacagcacggccaccgcctaccggaaggcacatcatcaccttctgag | |
| | tctgtatgcagagcatgagctggaacacgccctgaatgaaacctgtgaggcgcttgtc | |
| | cgggcaatgcatctgagcattctggtacaggaaaatccgctcgccaacaccaccggc | |
| | catcagggctacgtcgcaccggaaaaggctgtcatgcagcaggtgaaatcatcgctg | |
| | gaacagaaaattaaacagatgcaaatcagcctcaccggcgagccggttctccggctg | |
| | accggactgtcagcggcaacactcccgcacatggattatgaggtggcaggcacaccg | |
| | gcacagcgcaaggtgtggcaggacaaaatagaccagcagggagcagagcttaagg | |
| | ccagagggctgctgtcatgatttactgtccgtcgtgtggacatgttgctcacacccgtcg | |
| | cgcacatttcatggacgatggcaccaagataatgattgcacagtgccggaatatttattg | |
| | ctctgcgacatttgaagcgagtgaaagctttttctctgacagtaaagattcaggaatgga | |
| | atacatttcaggcaaacagagataccgcgattcactgacgtcagcctcctgcggtatga | |
| | aacgcccgaaaagaatgcttgttaccggatattgttgtcggagatgtaaaggccttgca | |
| | ctgtcaagaacatcgcggcgtctgtctcaggaagtcaccgagcgtttttatgtgtgcacg | |
| | gatccgggctgtggtctggtgtttaaaacgcttcagaccatcaaccgcttcattgtccgc | |
| | ccggtcacgccggacgaactggcagaacgcctgcatgaaaaacaggaactgccgcc | |
| | agtacggttaaaaacacaatcatattcgctgcgtctggaatgagggctgccggttaaca | |
| | ccggccgtcgccgcacaccgtatttttattcttcagcatgatgagaaagagataacgatg | |
| | gaaagcacagccttacagcaggcctttgacacctgtcagaataacaaagcagcatgg | |
| | ctgcaacgcaaaaatgagctggcagcggccgaacaggaatatctgcggcttctgtca | |
| | ggagaaggcagaaacgtcagtcgcctggacgaattacgcaatattatcgaagtcaga | |
| | aaatggcaggtgaatcaggccgccggtcgttatattcgttcgcatgaagccgttcagca | |
| | catcagcatccgcgaccggctgaatgattttatgcagcagcacggcacagcactggc | |
| | ggccgcactggcaccggagctgatgggctacagtgagctgacggccattgcccgaa | |
| | actgtgccatacagcgtgccacagatgccctgcgtgaagcccttctgtcctggcttgcg | |
| | aagggtgaaaaaattaattattccgcacaggatagcgacattttaacgaccatcggattc | |
| | aggcctgacgtggcttcggtggatgacagccgtgaaaaattcacccctgcgcagaac | |
| | atgattttttcgcgtaaaagtgcgcaactggcatcacgtcagtcagtgtaaaattccccg | |
| | aaaatccgcccgtttttactgaaaaaagccatgcatcgataaggtgcatggcttt | |
| pP4- | gcatgcgttttcctgcctcattttctgcaaaccgcgccattcccggcgcggtctgagcgt | pP4-c2 |
| LacIq- | gtcagtgcaactgcattaaaaccgccccgcaaagcgggcgggcgaggcggggaaa | |
| Large | gcaccgcgcgcaaaccgacaagttagttaattatttgtgtagtcaaagtgccttcagtac | |
| | atacctcgttaatacattggagcataatgaagaaaatctatggcctatggtCCAAAA | |
| | CTGTCTTTTTTGATGGCACTATCCTGAAAAATATGCA | |
| | AAAAATAGATTGATGTAAGGTGGTTCTTGTCAGTGT | |
| | CGCAAGATCCTTAAGAATTCGTGGCATGAGAGAGTT | |
| | AAAGGGCGTCCCTATCAGTGATAGAGATTGACATCC | |
| | CTATCAGTGATAGAGATACTGAGCACATCAGCAGGA | |
| | CGCACTGACCGAATTCGAAAGAGGAGAAAGGAGATc | |
| | atatgctctccacactccgccgcactctatttgcgctgctggcttgtgcgtcttttatcgtc | |
| | catgccgatccgccggccaccgtgtataaatatgatagccgcccgccggaagatgtgt | |
| | ttcagaatggcttcaccgcctggggcaacaatgataacgtgctggaccacctgaccgg | |
| | tcgtagcggtcaggtgggtagcagcaacagtgcctttgtgagcaccagcagcagccg | |
| | tcgctataccgaagtgtacctggaacatcgcatgcaggaagcagtggaagcagaacg | |
| | tgcaggccgtggcaccggccattttattggctacatttatgaggtgcgcgccgataataa | |
| | cttttacggcgccgccagcagctactttgaatacgtggacacctacggcgacaatgcc | |
| | ggtcgtatcctggcaggtgccctggcaacctatcagagcggttatctggcccatcgcc | |
| | gcattccgccggaaaatattcgccgtgtgacccgcgtgtatcataacggcattaccggc | |
| | gaaaccaccaccaccgagtatagcaatgcccgctatgttagccagcagacccgcgcc | |
| | aatccgaatccgtataccagcggcggtcatcatcaccaccaccattaataactcCTT | |
| | TAGCGTGGATAAAAAATAAtaaCCCGGGCTCGGTACC | |
| | AAATTCCAGAAAAGAGGCCTCCCGAAAGGGGGGCC | |
| | TTTTTTCGTTTTGGTCCTGGTAAATAGAAACGGAACT | |
| | TTACATATTGAATACCGGAGATATAATTCGTTTATGC | |
| | CACATAATCATCAATACATCCGTAACCCGCCCGAAA | |
| | CGGAAAAACTGAAGAAAAACCCCATTATCTTAGCCT | |
| | AAAAAAACCTTCTCTTTGGAACTTTCAGTAATACGCT | |
| | TAACTGCTCATTGCTATATTGAAGTACGGATTAGAA | |
| | GCCGCCGAGCGGGTGACAGCCCTCCGAAGGAAGAC | |
| | TCTCCTCCGTGCGTCCTCGTCTTCACCGGTCGCGTTC | |
| | CTGAAACGCAGATGTGCCTCGCGCCGCACTGCTCCG | |
| | AACAATAAAGATTCTACAATACTAGCTTTTATGGTT | |
| | ATGAAGAGGAAAAATTGGCAGTAACCTGGCCCCAC | |
| | AAACCTTCAAATGAACGAATCAAATTAACAACCATA | |
| | GGATGATAATGCGATTAGTTTTTTAGCCTTATTTCTG | |
| | GGGTAATTAATCAGCGAAGCGATGATTTTTGATCTA | |
| | TTAACAGATATATAAATGCAAAAACTGCATAACCAC | |
| | TTTAACTAATACTTTCAACATTTTCGGTTTGTATTAC | |
| | TTCTTATTCAAATGTAATAAAAGTATCAACAAAAAA | |
| | TTGTTAATATACCTCTATACTTTAACGTCAAGGAGGA | |
| | AACTAGACCCGCCGCCACCATGGAGATGAGTGACGC | |
| | GGCACCGAGCTTGTCTAACCTTTTCTATGATCCGACC | |
| | TACAACCCTGGGCAAAGTACGATTGATTACAAAGAC | |
| | GATGACGACAAGGCCGAGATCTATAATAAAGACGG | |
| | AAATAAATTGGATTTGTTCGGTAAAGTCGACGGACT | |
| | TCACTACTTCTCTGATGACAAGGGAAGTGACGGAGA | |
| | CCAGACTTACATGAGAATCGGATTCAAGGGTGAGAC | |
| | CCAAGTTAACGACCAGCTTACCGGATACGGTCAGTG | |
| | GGAGTACCAGATCCAAGGAAACCAGACCGAGGGTA | |
| | GTAACGACTCTTGGACCAGAGTCGCTTTCGCCGGTTT | |
| | GAAGTTCGCTGACGCTGGTAGTTTCGACTACGGAAG | |
| | GAATTACGGAGTCACCTACGACGTTACCTCTTGGAC | |
| | CGACGTCCTTCCAGAATTCGGTGGAGATACCTACGG | |
| | TGCCGACAACTTCATGCAGCAAAGGGGTAACGGTTA | |
| | TGCCACCTATAGAAACACCGACTTCTTCGGTTTGGTC | |
| | GACGGTTTGGACTTCGCCTTGCAGTACCAAGGTAAG | |
| | AATGGTAGTGTTTCTGGTGAAAATACCAATGGTAGA | |
| | TCTTTGCTTAATCAGAACGGAGACGGTTACGGTGGT | |
| | TCTTTGACCTACGCCATCGGTGAAGGTTTCTCTGTCG | |
| | GAGGTGCCATTACCACCAGTAAGAGAACCGCCGACC | |
| | AGAACAATACCGCCAACGCCAGATTGTACGGAAAC | |
| | GGTGATAGAGCCACCGTCTACACCGGTGGTTTGAAA | |
| | TACGACGCTAACAACATCTACCTTGCTGCCCAGTAC | |
| | AGTCAGACCTACAATGCCACTAGATTCGGAACCAGT | |
| | AACGGTAGTAACCCAAGTACCTCTTACGGATTTGCC | |
| | AACAAGGCCCAGAACTTTGAGGTCGTTGCCCAGTAC | |
| | CAGTTCGACTTCGGTCTTAGGCCAAGTGTCGCTTATC | |
| | TTCAGTCTAAGGGTAAGGACATCTCTAACGGTTACG | |
| | GAGCCTCTTACGGTGACCAAGATATCGTCAAGTACG | |
| | TCGACGTCGGAGCTACCTACTACTTTAATAAAAACA | |
| | TGAGTACTTACGTCGATTATAAGATCAATTTGTTGGA | |
| | CAAAAACGACTTCACTAGAGATGCCGGTATCAATcta | |
| | gcataaccccttggggcctctaaacgggtcttgaggggttttttgagacaaacaaaaga | |
| | atggaatcaaagttaaTGCTAGCAGCcGCCGctgcaggcatgcaagcttg | |
| | cggccgcgtcgtgactgggaaaaccctggcgactagtcttggactcctgttgatagatc | |
| | cagtaatgacctcagaactccatctggatttgttcagaacgctcggttgccgccgggcg | |
| | ttttttattggtgagaatccaggggtccccaataattacgatttaaatttgtgtctcaaaatct | |
| | ctgatgttacattgcacaagataaaaatatatcatcatgaacaataaaactgtctgcttaca | |
| | taaacagtaatacaaggggtgttatgagccatattcagcgtgaaacgagctgtagccgt | |
| | ccgcgtctgaacagcaacatggatgcggatctgtatggctataaatgggcgcgtgata | |
| | acgtgggtcagagcggcgcgaccatttatcgtctgtatggcaaaccggatgcgccgg | |
| | aactgtttctgaaacatggcaaaggcagcgtggcgaacgatgtgaccgatgaaatggt | |
| | gcgtctgaactggctgaccgaatttatgccgctgccgaccattaaacattttattcgcacc | |
| | ccggatgatgcgtggctgctgaccaccgcgattccgggcaaaaccgcgtttcaggtg | |
| | ctggaagaatatccggatagcggcgaaaacattgtggatgcgctggccgtgtttctgc | |
| | gtcgtctgcatagcattccggtgtgcaactgcccgtttaacagcgatcgtgtgtttcgtct | |
| | ggcccaggcgcagagccgtatgaacaacggcctggtggatgcgagcgattttgatga | |
| | tgaacgtaacggctggccggtggaacaggtgtggaaagaaatgcataaactgctgcc | |
| | gtttagcccggatagcgtggtgacccacggcgattttagcctggataacctgattttcga | |
| | tgaaggcaaactgattggctgcattgatgtgggccgtgtgggcattgcggatcgttatc | |
| | aggatctggccattctgtggaactgcctgggcgaatttagcccgagcctgcaaaaacg | |
| | tctgtttcagaaatatggcattgataatccggatatgaacaaactgcaatttcatctgatgc | |
| | tggatgaatttttctaacttggaagtaagaatggtgccgaaggccggactcaaacatca | |
| | aaataagttaatgataaaaaacaaataataaaacacaacaatgaaatatgcccccttttgt | |
| | gcccccactgtttttctgaccaatctattttcagcccatcaataaatcggaaagttaaatca | |
| | tttttaatcagtaagtttggatccgtagctcggatccaaaccagtgcatcttttatccacata | |
| | aaaaattttttttcgaaagaactgttcacactgttcacctttctgttttctccttttatttcagag | |
| | tgataggtggtgaataatgggtgaagggtgaacattcgattcttcacctccggcattctg | |
| | ccgatgtgactcataccggtgattaatcctccgcactgaaatcactcaggaagaaaaaa | |
| | gttttttttgatttgattgttcacactgttcacctttcgtttttctcttttaatttcagtgtgataac | |
| | gggtgaatatacggtgaagggtgaacagtggattgttcaccttcgggggatatcggga | |
| | taaaaaaagaccggcagatgccggtcaggtgggtcaggctgttgtagggtcgtcacat | |
| | tttggcagccagtcgccgtagctttcctctttcagcgtcaggttggtctgtatcccctgttt | |
| | ggtatggcgtttctcgtaattcagtccgtattccttcagcatcaccggcagccccagccc | |
| | gaacattttcagactgagtacattccggtagccgtttgcctccatgtaggccagataggc | |
| | gtgatagaggtatttacggtaattgcgcgggatgatactggcgttccccatatacatgcc | |
| | gctggtctgcggcagggtttccagatagccgataaaatcaaacgtcgggtcggcatcc | |
| | cgtttgatgttcagtgcctcgtctgagttctgctgggactgaagcagtgaccgggcgag | |
| | catcgggtcgctgaacttctgcatcaggtgacgcacgatgaccgccagctcgcgggt | |
| | gattttgtccttaagctgcgggtcgcgctcctgcggggctatctgttccgggaagtgaat | |
| | aatcacccgtcggcgtgacacgccgccgctgcggtcggtgaagcgcatcgggttatt | |
| | gttcacggccagaatcaccgccgggatgtgcgtggagtacgcatcccggtatttcggg | |
| | tcaacggacaccgcatcgccgccggtgatggccttgagtccggcaccgtcgccgctc | |
| | catttttcctggtccggcaggcgtatcagtgagaagccagttaacgcggcacgttcacg | |
| | cggggattccagcgtctcgatggtggccgacgtggcgttatcctccccggccagcag | |
| | ggtggctatttcggccatgatacttttgccgctgccgccgggaccggtcacctccagaa | |
| | agagctgccagtcgtagcggtttgccagcaccataaacagtgcagccagaatcacgtc | |
| | gcgtttttccgcacggccaccggcggcacggtcaagccagcgccagaaggcgggg | |
| | gcgtgggtttccagcgtttcaccgtccaccggcggggtgaaatccacatcgcacagg | |
| | gtgcgcatccagtgtgacggactgtgcgggtggaacgtgccgttctgcgtgtcgagca | |
| | cgccgttacgaaagccaatcaggcggcgggagggggcttcctgctgcggaataatca | |
| | gcttcagggtatccaccacggaggccaccttcccggaggagaacggcgcacgcaga | |
| | cgctgaaacagcccggccacatcccgggcaaagtcctgtggcggcagcaccttcca | |
| | gacaccattttcatagcgggacagaagctggccgttggcatcgaccgcgagcgcctc | |
| | gccgtaatgctcatagatacgcatggccttttcgctggtactcatggcggaaaactccg | |
| | cttcgctcatggtgtcgaacgggctttcagccggtggccggatggcatcgtaaatggc | |
| | cttacgggtggcctccccgccgtactgcgtgaaggcatcattccagtcaccgaagacc | |
| | ggcggcagggcaacaacaccttcacacgcatctgcggctgcggcggcttttttctggc | |
| | cgtcaccactgaggtcacggtcagcggcaaggacaatctgacaggcggggtgcttct | |
| | gccgggcaaggctggccagagaaaggaggttcacggaagaaagcgccaccatcac | |
| | cgtttcaccggtcaggtgatgtacggtaagtgcggtcgcgtatccctccgctatccaca | |
| | gacgttttccggcctgattctgtccttcaagggtgtgacaggtgcccctgacctgtccgc | |
| | ctttcagggtgcgcttacggccgtcagcactgattaactgaaggttaaccagttcgccg | |
| | ctgtcgtcatacagtggcaccacaaggtcaccggcgcgccagctcacgccaccggct | |
| | ctgtgtgtgccggtcagcatccggcattcccggccgggaaagcccttgcgggtcaggt | |
| | aggcgttaccggttccggtacgggttttcgccatcagggtttgtgccagtgcggcggc | |
| | gttcttccgggcagcgtctgtttcatcaacggcggcggtcgtcactgccgggtcagcc | |
| | ggtggcaggctgccggtcacggcagccacctttgcggccgcgtcggacggggaaa | |
| | caccaaaaaccttttcaaccagtttcaggccgtcaccggcaccacactgattgcagtac | |
| | caggtgccgcgcccctccctgtcatcaaaacggaagcggtcactcccgccacagacc | |
| | ggacagggctgatgacggttcttcagcacctgaatccccagcgccgggagaatacgc | |
| | ggccagtggccgagcgcatggctgacggtggcggttacgttcattttcatggtgttgttc | |
| | tccttcagtgcagtaccggcgcttttatgtgacgggcacagagttcatccatcacaacca | |
| | gcccgagaaaggacagcgacggcgcggccttcagggggccggattccattaaatctt | |
| | ccagcagggcacaggctatctgacgccctttttcctcaccgtgctggcgcagataaaa | |
| | gccttccagctcagcggcgatggccgcctccagtgactcaagggtgagatgcgggta | |
| | gcggtgctgacgttcgcacacggtcagccaggcacaggcgacagcgcgacggtaa | |
| | agggcagcgcgtaagacgggcggtaagggtgttttcatttgcttttctccctgtgacaga | |
| | tgactgcattccgtgccggttgcattaactgataaggcatatctgcgtctcctgaagacgt | |
| | gcgtatccctgcgcgaatacgcacatttaatttttcgggggtcgttttttaattacagataat | |
| | tgcggtaactgttatccggggtggtttccgggtcaggctccgtgcggggaatttcccgc | |
| | cattcccgcgccaccggtgctgcccggctgaccggaacagtgtcctgcgggtaaatat | |
| | ccagatatttttcccgccatttctgtaattccgggtctccggccatttctttcagtaccgcat | |
| | gccggtttacggggctgcgtttaaacaggtcaggacggtcacaggtaaattcccgcag | |
| | aaaacgccccagcgggatgtctgtggtgcgtccgtcagcgaggatacgcacaaggat | |
| | actgaatttacggcggtacgggttccagacaatgtccgggcagcggtacggcatttcc | |
| | cacggaataccgtcttccagaatgccgaccacggccacatcgggaaaaccggcaga | |
| | acggtaaatctcaccgggctggggaaaatcaaacatgcgtcctgtctccccggtctttct | |
| | gctgggcgagaaaatcgcggcacaggcctttggctttcagctcattcagcacaaaatc | |
| | aatatcttcattcaggtagctgaaaatatgtggaatgtagagctgatgcaggccggaga | |
| | gttcacggtgaatcaaatcacccccaacaaaccgggatacggcgctggcgcggttga | |
| | gcttatggtaagcctcaatgctgaggtgttcacgggcgtcatgacgcgctgagacggt | |
| | ctgaggggcttttttattacgcacgggacacctccaccaccggcagacgggcagcaa | |
| | gggagagcacatagtcacggacaagggaacggcgggcactgcgttcatcaccggc | |
| | gacggtgcgaagcatacagatacggggatgacggtctgcgcgacggacagccgca | |
| | aacacaaagacaaattcagggtgtgagggggtaagggttgtagccatgatggcagcc | |
| | tcctgtgaatagcaaataacgctatcgccggagttctcacgctcgatggcgatagccca | |
| | gacgggggtgagaataccggcttcacaggataccggccagcccggaggctgcccc | |
| | gcctgagctaccattgactctgcggcataatgagcggacgcgggcaggatgcacgga | |
| | atgccatctgcacgactgaccacacaccacaccataatctggcgctctgtggcattgatt | |
| | gcgacacaaaaaaagacgcgtggcgcgtcatatgtcgcctgtgaattgctcgggttct | |
| | cacgcccggctgccgattttgcggcaggcgaaaaactatatccgcaaatgccggaaa | |
| | aaggcaagccagaaaaagggagtttttgcagagcgggcatcatcatgcgtcgtaccc | |
| | ccgtttgcgtccggcaatgcgtccggccatccatgcggtgacttcagagtgcagccag | |
| | gccacatttttaccgccaagactcacctgcggcggaaattcccccttacggatgagttc | |
| | gtagatggtcgagcgtgacaggccgcacaggtgcatcacttccggcacacgtaaaaa | |
| | acgctcctgcgtgatgtccggcagcggcatcagtggcgtcactggggcgggagacg | |
| | gggaagaaaaaacagcttgcatcgggctacctcgttaatgtccatacagcaccggata | |
| | agtccgtccggcttcgggtagcgctttattttgtgaatattttcagcagacgcaacaggg | |
| | gggatttgttcaggctgtcttacaatggctgtgtgttttttgttcatctccacttaaagtcattt | |
| | aaagccacttaaagcaatttgtaatttttatagtgaaatacaaatcgtttcttcttattcattcc | |
| | cggcgaattaataaaaacaaacagtagtaaacagcacaaaaagcccatcaacgggtg | |
| | aacagtggtgaacagacggtgaacagtcattactgcgattgttcaccctttaacttactgt | |
| | attacttatcttttttattaaggtgaacagaggtgaacagtaaaatataaaaaaacaaaca | |
| | gtaagccggtttttcctgcgaccttttcctggcttgccggtctgaggatgagtctcctgtgt | |
| | cagggctggcacatctgcaatgcgtcgtgttgttgtccggtgtacgtcacaattttcttaa | |
| | cctgaagtgacgaggagccggaaaatgtctgaccacactatccctgaatatctgcaac | |
| | ccgcactggcacaactggaaaaggccagagccgcccatcttgagaacgcccgcctg | |
| | atggatgagaccgtcacggccattgaacgggcagagcaggaaaaaaatgcgctggc | |
| | gcaggccgacggaaacgacgctgacgactggcgcacggcctttcgtgcagccggtg | |
| | gtgtcctgagcgacgagctgaaacagcgccacattgagcgcgtggcacgccgggag | |
| | ctggtacaggaatatgacaatctggccgtggtgctgaatttcgaacgtgaacgcctgaa | |
| | aggggcgtgtgacagcacggccaccgcctaccggaaggcacatcatcaccttctgag | |
| | tctgtatgcagagcatgagctggaacacgccctgaatgaaacctgtgaggcgcttgtc | |
| | cgggcaatgcatctgagcattctggtacaggaaaatccgctcgccaacaccaccggc | |
| | catcagggctacgtcgcaccggaaaaggctgtcatgcagcaggtgaaatcatcgctg | |
| | gaacagaaaattaaacagatgcaaatcagcctcaccggcgagccggttctccggctg | |
| | accggactgtcagcggcaacactcccgcacatggattatgaggtggcaggcacaccg | |
| | gcacagcgcaaggtgtggcaggacaaaatagaccagcagggagcagagcttaagg | |
| | ccagagggctgctgtcatgatttactgtccgtcgtgtggacatgttgctcacacccgtcg | |
| | cgcacatttcatggacgatggcaccaagataatgattgcacagtgccggaatatttattg | |
| | ctctgcgacatttgaagcgagtgaaagctttttctctgacagtaaagattcaggaatgga | |
| | atacatttcaggcaaacagagataccgcgattcactgacgtcagcctcctgcggtatga | |
| | aacgcccgaaaagaatgcttgttaccggatattgttgtcggagatgtaaaggccttgca | |
| | ctgtcaagaacatcgcggcgtctgtctcaggaagtcaccgagcgtttttatgtgtgcacg | |
| | gatccgggctgtggtctggtgtttaaaacgcttcagaccatcaaccgcttcattgtccgc | |
| | ccggtcacgccggacgaactggcagaacgcctgcatgaaaaacaggaactgccgcc | |
| | agtacggttaaaaacacaatcatattcgctgcgtctggaatgagggctgccggttaaca | |
| | ccggccgtcgccgcacaccgtatttttattcttcagcatgatgagaaagagataacgatg | |
| | gaaagcacagccttacagcaggcctttgacacctgtcagaataacaaagcagcatgg | |
| | ctgcaacgcaaaaatgagctggcagcggccgaacaggaatatctgcggcttctgtca | |
| | ggagaaggcagaaacgtcagtcgcctggacgaattacgcaatattatcgaagtcaga | |
| | aaatggcaggtgaatcaggccgccggtcgttatattcgttcgcatgaagccgttcagca | |
| | catcagcatccgcgaccggctgaatgattttatgcagcagcacggcacagcactggc | |
| | ggccgcactggcaccggagctgatgggctacagtgagctgacggccattgcccgaa | |
| | actgtgccatacagcgtgccacagatgccctgcgtgaagcccttctgtcctggcttgcg | |
| | aagggtgaaaaaattaattattccgcacaggatagcgacattttaacgaccatcggattc | |
| | aggcctgacgtggcttcggtggatgacagccgtgaaaaattcacccctgcgcagaac | |
| | atgattttttcgcgtaaaagtgcgcaactggcatcacgtcagtcagtgtaaaattccccg | |
| | aaaatccgcccgtttttactgaaaaaagccatgcatcgataaggtgcatggcttt | |
| pP4- | gcatgcgttttcctgcctcattttctgcaaaccgcgccattcccggcgcggtctgagcgt | pP4-c2 |
| LacIq- | gtcagtgcaactgcattaaaaccgccccgcaaagcgggcgggcgaggcggggaaa | |
| Large | gcaccgcgcgcaaaccgacaagttagttaattatttgtgtagtcaaagtgccttcagtac | |
| | atacctcgttaatacattggagcataatgaagaaaatctatggcctatggtCCAAAA | |
| | CTGTCTTTTTTGATGGCACTATCCTGAAAAATATGCA | |
| | AAAAATAGATTGATGTAAGGTGGTTCTTGTCAGTGT | |
| | CGCAAGATCCTTAAGAATTCGTGGCATGAGAGAGTT | |
| | AAAGGGCGTGCGTTCCTGTATTTCCGGAGGGCCCTA | |
| | ATACTCGAATGGTGCAAAACCTTTCGCGGTATGGCA | |
| | TGATAGCGCCGAATTCGAAAGAGGAGAAAGGAGAT | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnntaaCCCGGGCTCGGTACCAAATTCCAGAAAA | |
| | GAGGCCTCCCGAAAGGGGGGCCTTTTTTCGTTTTGGT | |
| | CCGAGCTCTGGTAAATAGAAACGGAACTTTACATAT | |
| | TGAATACCGGAGATATAATTCGTTTATGCCACATAA | |
| | TCATCAATACATCCGTAACCCGCCCGAAACGGAAAA | |
| | ACTGAAGAAAAACCCCATTATCTTAGCCTAAAAAAA | |
| | CCTTCTCTTTGGAACTTTCAGTAATACGCTTAACTGC | |
| | TCATTGCTATATTGAAGTACGGATTAGAAGCCGCCG | |
| | AGCGGGTGACAGCCCTCCGAAGGAAGACTCTCCTCC | |
| | GTGCGTCCTCGTCTTCACCGGTCGCGTTCCTGAAACG | |
| | CAGATGTGCCTCGCGCCGCACTGCTCCGAACAATAA | |
| | AGATTCTACAATACTAGCTTTTATGGTTATGAAGAG | |
| | GAAAAATTGGCAGTAACCTGGCCCCACAAACCTTCA | |
| | AATGAACGAATCAAATTAACAACCATAGGATGATAA | |
| | TGCGATTAGTTTTTTAGCCTTATTTCTGGGGTAATTA | |
| | ATCAGCGAAGCGATGATTTTTGATCTATTAACAGAT | |
| | ATATAAATGCAAAAACTGCATAACCACTTTAACTAA | |
| | TACTTTCAACATTTTCGGTTTGTATTACTTCTTATTCA | |
| | AATGTAATAAAAGTATCAACAAAAAATTGTTAATAT | |
| | ACCTCTATACTTTActagcataaccccttggggcctctaaacgggtcttga | |
| | ggggttttttgagacaaacaaaagaatggaatcaaagttaaTGCTAGCAGCc | |
| | GCCGctgcaggcatgcaagcttgcggccgcgtcgtgactgggaaaaccctggcg | |
| | actagtcttggactcctgttgatagatccagtaatgacctcagaactccatctggatttgtt | |
| | cagaacgctcggttgccgccgggcgttttttattggtgagaatccaggggtccccaata | |
| | attacgatttaaatttgtgtctcaaaatctctgatgttacattgcacaagataaaaatatatc | |
| | atcatgaacaataaaactgtctgcttacataaacagtaatacaaggggtgttatgagcca | |
| | tattcagcgtgaaacgagctgtagccgtccgcgtctgaacagcaacatggatgcggat | |
| | ctgtatggctataaatgggcgcgtgataacgtgggtcagagcggcgcgaccatttatc | |
| | gtctgtatggcaaaccggatgcgccggaactgtttctgaaacatggcaaaggcagcgt | |
| | ggcgaacgatgtgaccgatgaaatggtgcgtctgaactggctgaccgaatttatgccg | |
| | ctgccgaccattaaacattttattcgcaccccggatgatgcgtggctgctgaccaccgc | |
| | gattccgggcaaaaccgcgtttcaggtgctggaagaatatccggatagcggcgaaaa | |
| | cattgtggatgcgctggccgtgtttctgcgtcgtctgcatagcattccggtgtgcaactg | |
| | cccgtttaacagcgatcgtgtgtttcgtctggcccaggcgcagagccgtatgaacaac | |
| | ggcctggtggatgcgagcgattttgatgatgaacgtaacggctggccggtggaacag | |
| | gtgtggaaagaaatgcataaactgctgccgtttagcccggatagcgtggtgacccacg | |
| | gcgattttagcctggataacctgattttcgatgaaggcaaactgattggctgcattgatgt | |
| | gggccgtgtgggcattgcggatcgttatcaggatctggccattctgtggaactgcctgg | |
| | gcgaatttagcccgagcctgcaaaaacgtctgtttcagaaatatggcattgataatccgg | |
| | atatgaacaaactgcaatttcatctgatgctggatgaatttttctaacttggaagtaagaat | |
| | ggtgccgaaggccggactcaaacatcaaaataagttaatgataaaaaacaaataataa | |
| | aacacaacaatgaaatatgcccccttttgtgcccccactgtttttctgaccaatctattttca | |
| | gcccatcaataaatcggaaagttaaatcatttttaatcagtaagtttggatccgtagctcg | |
| | gatccaaaccagtgcatcttttatccacataaaaaattttttttcgaaagaactgttcacact | |
| | gttcacctttctgttttctccttttatttcagagtgataggtggtgaataatgggtgaagggt | |
| | gaacattcgattcttcacctccggcattctgccgatgtgactcataccggtgattaatcct | |
| | ccgcactgaaatcactcaggaagaaaaaagttttttttgatttgattgttcacactgttcac | |
| | ctttcgtttttctcttttaatttcagtgtgataacgggtgaatatacggtgaagggtgaacag | |
| | tggattgttcaccttcgggggatatcgggataaaaaaagaccggcagatgccggtcag | |
| | gtgggtcaggctgttgtagggtcgtcacattttggcagccagtcgccgtagctttcctctt | |
| | tcagcgtcaggttggtctgtatcccctgtttggtatggcgtttctcgtaattcagtccgtatt | |
| | ccttcagcatcaccggcagccccagcccgaacattttcagactgagtacattccggtag | |
| | ccgtttgcctccatgtaggccagataggcgtgatagaggtatttacggtaattgcgcgg | |
| | gatgatactggcgttccccatatacatgccgctggtctgcggcagggtttccagatagc | |
| | cgataaaatcaaacgtcgggtcggcatcccgtttgatgttcagtgcctcgtctgagttct | |
| | gctgggactgaagcagtgaccgggcgagcatcgggtcgctgaacttctgcatcaggt | |
| | gacgcacgatgaccgccagctcgcgggtgattttgtccttaagctgcgggtcgcgctc | |
| | ctgcggggctatctgttccgggaagtgaataatcacccgtcggcgtgacacgccgcc | |
| | gctgcggtcggtgaagcgcatcgggttattgttcacggccagaatcaccgccgggatg | |
| | tgcgtggagtacgcatcccggtatttcgggtcaacggacaccgcatcgccgccggtg | |
| | atggccttgagtccggcaccgtcgccgctccatttttcctggtccggcaggcgtatcagt | |
| | gagaagccagttaacgcggcacgttcacgcggggattccagcgtctcgatggtggcc | |
| | gacgtggcgttatcctccccggccagcagggtggctatttcggccatgatacttttgcc | |
| | gctgccgccgggaccggtcacctccagaaagagctgccagtcgtagcggtttgccag | |
| | caccataaacagtgcagccagaatcacgtcgcgtttttccgcacggccaccggcggc | |
| | acggtcaagccagcgccagaaggcgggggcgtgggtttccagcgtttcaccgtccac | |
| | cggcggggtgaaatccacatcgcacagggtgcgcatccagtgtgacggactgtgcg | |
| | ggtggaacgtgccgttctgcgtgtcgagcacgccgttacgaaagccaatcaggcggc | |
| | gggagggggcttcctgctgcggaataatcagcttcagggtatccaccacggaggcca | |
| | ccttcccggaggagaacggcgcacgcagacgctgaaacagcccggccacatcccg | |
| | ggcaaagtcctgtggcggcagcaccttccagacaccattttcatagcgggacagaag | |
| | ctggccgttggcatcgaccgcgagcgcctcgccgtaatgctcatagatacgcatggcc | |
| | ttttcgctggtactcatggcggaaaactccgcttcgctcatggtgtcgaacgggctttca | |
| | gccggtggccggatggcatcgtaaatggccttacgggtggcctccccgccgtactgc | |
| | gtgaaggcatcattccagtcaccgaagaccggcggcagggcaacaacaccttcacac | |
| | gcatctgcggctgcggcggcttttttctggccgtcaccactgaggtcacggtcagcgg | |
| | caaggacaatctgacaggcggggtgcttctgccgggcaaggctggccagagaaagg | |
| | aggttcacggaagaaagcgccaccatcaccgtttcaccggtcaggtgatgtacggtaa | |
| | gtgcggtcgcgtatccctccgctatccacagacgttttccggcctgattctgtccttcaag | |
| | ggtgtgacaggtgcccctgacctgtccgcctttcagggtgcgcttacggccgtcagca | |
| | ctgattaactgaaggttaaccagttcgccgctgtcgtcatacagtggcaccacaaggtc | |
| | accggcgcgccagctcacgccaccggctctgtgtgtgccggtcagcatccggcattc | |
| | ccggccgggaaagcccttgcgggtcaggtaggcgttaccggttccggtacgggttttc | |
| | gccatcagggtttgtgccagtgcggcggcgttcttccgggcagcgtctgtttcatcaac | |
| | ggcggcggtcgtcactgccgggtcagccggtggcaggctgccggtcacggcagcc | |
| | acctttgcggccgcgtcggacggggaaacaccaaaaaccttttcaaccagtttcaggc | |
| | cgtcaccggcaccacactgattgcagtaccaggtgccgcgcccctccctgtcatcaaa | |
| | acggaagcggtcactcccgccacagaccggacagggctgatgacggttcttcagcac | |
| | ctgaatccccagcgccgggagaatacgcggccagtggccgagcgcatggctgacg | |
| | gtggcggttacgttcattttcatggtgttgttctccttcagtgcagtaccggcgcttttatgt | |
| | gacgggcacagagttcatccatcacaaccagcccgagaaaggacagcgacggcgc | |
| | ggccttcagggggccggattccattaaatcttccagcagggcacaggctatctgacgc | |
| | cctttttcctcaccgtgctggcgcagataaaagccttccagctcagcggcgatggccgc | |
| | ctccagtgactcaagggtgagatgcgggtagcggtgctgacgttcgcacacggtcag | |
| | ccaggcacaggcgacagcgcgacggtaaagggcagcgcgtaagacgggcggtaa | |
| | gggtgttttcatttgcttttctccctgtgacagatgactgcattccgtgccggttgcattaac | |
| | tgataaggcatatctgcgtctcctgaagacgtgcgtatccctgcgcgaatacgcacattt | |
| | aatttttcgggggtcgttttttaattacagataattgcggtaactgttatccggggggtttc | |
| | cgggtcaggctccgtgcggggaatttcccgccattcccgcgccaccggtgctgcccg | |
| | gctgaccggaacagtgtcctgcgggtaaatatccagatatttttcccgccatttctgtaatt | |
| | ccgggtctccggccatttctttcagtaccgcatgccggtttacggggctgcgtttaaaca | |
| | ggtcaggacggtcacaggtaaattcccgcagaaaacgccccagcgggatgtctgtgg | |
| | tgcgtccgtcagcgaggatacgcacaaggatactgaatttacggcggtacgggttcca | |
| | gacaatgtccgggcagcggtacggcatttcccacggaataccgtcttccagaatgccg | |
| | accacggccacatcgggaaaaccggcagaacggtaaatctcaccgggctggggaa | |
| | aatcaaacatgcgtcctgtctccccggtctttctgctgggcgagaaaatcgcggcacag | |
| | gcctttggctttcagctcattcagcacaaaatcaatatcttcattcaggtagctgaaaatat | |
| | gtggaatgtagagctgatgcaggccggagagttcacggtgaatcaaatcacccccaa | |
| | caaaccgggatacggcgctggcgcggttgagcttatggtaagcctcaatgctgaggtg | |
| | ttcacgggcgtcatgacgcgctgagacggtctgaggggcttttttattacgcacgggac | |
| | acctccaccaccggcagacgggcagcaagggagagcacatagtcacggacaaggg | |
| | aacggcgggcactgcgttcatcaccggcgacggtgcgaagcatacagatacgggga | |
| | tgacggtctgcgcgacggacagccgcaaacacaaagacaaattcagggtgtgaggg | |
| | ggtaagggttgtagccatgatggcagcctcctgtgaatagcaaataacgctatcgccg | |
| | gagttctcacgctcgatggcgatagcccagacgggggtgagaataccggcttcacag | |
| | gataccggccagcccggaggctgccccgcctgagctaccattgactctgcggcataa | |
| | tgagcggacgcgggcaggatgcacggaatgccatctgcacgactgaccacacacca | |
| | caccataatctggcgctctgtggcattgattgcgacacaaaaaaagacgcgtggcgcg | |
| | tcatatgtcgcctgtgaattgctcgggttctcacgcccggctgccgattttgcggcaggc | |
| | gaaaaactatatccgcaaatgccggaaaaaggcaagccagaaaaagggagtttttgc | |
| | agagcgggcatcatcatgcgtcgtacccccgtttgcgtccggcaatgcgtccggccat | |
| | ccatgcggtgacttcagagtgcagccaggccacatttttaccgccaagactcacctgc | |
| | ggcggaaattcccccttacggatgagttcgtagatggtcgagcgtgacaggccgcac | |
| | aggtgcatcacttccggcacacgtaaaaaacgctcctgcgtgatgtccggcagcggc | |
| | atcagtggcgtcactggggcgggagacggggaagaaaaaacagcttgcatcgggct | |
| | acctcgttaatgtccatacagcaccggataagtccgtccggcttcgggtagcgctttattt | |
| | tgtgaatattttcagcagacgcaacaggggggatttgttcaggctgtcttacaatggctgt | |
| | gtgttttttgttcatctccacttaaagtcatttaaagccacttaaagcaatttgtaatttttatag | |
| | tgaaatacaaatcgtttcttcttattcattcccggcgaattaataaaaacaaacagtagtaa | |
| | acagcacaaaaagcccatcaacgggtgaacagtggtgaacagacggtgaacagtca | |
| | ttactgcgattgttcaccctttaacttactgtattacttatcttttttattaaggtgaacagagg | |
| | tgaacagtaaaatataaaaaaacaaacagtaagccggtttttcctgcgaccttttcctgg | |
| | cttgccggtctgaggatgagtctcctgtgtcagggctggcacatctgcaatgcgtcgtgt | |
| | tgttgtccggtgtacgtcacaattttcttaacctgaagtgacgaggagccggaaaatgtc | |
| | tgaccacactatccctgaatatctgcaacccgcactggcacaactggaaaaggccaga | |
| | gccgcccatcttgagaacgcccgcctgatggatgagaccgtcacggccattgaacgg | |
| | gcagagcaggaaaaaaatgcgctggcgcaggccgacggaaacgacgctgacgact | |
| | ggcgcacggcctttcgtgcagccggtggtgtcctgagcgacgagctgaaacagcgc | |
| | cacattgagcgcgtggcacgccgggagctggtacaggaatatgacaatctggccgtg | |
| | gtgctgaatttcgaacgtgaacgcctgaaaggggcgtgtgacagcacggccaccgcc | |
| | taccggaaggcacatcatcaccttctgagtctgtatgcagagcatgagctggaacacg | |
| | ccctgaatgaaacctgtgaggcgcttgtccgggcaatgcatctgagcattctggtacag | |
| | gaaaatccgctcgccaacaccaccggccatcagggctacgtcgcaccggaaaaggc | |
| | tgtcatgcagcaggtgaaatcatcgctggaacagaaaattaaacagatgcaaatcagc | |
| | ctcaccggcgagccggttctccggctgaccggactgtcagcggcaacactcccgcac | |
| | atggattatgaggtggcaggcacaccggcacagcgcaaggtgtggcaggacaaaat | |
| | agaccagcagggagcagagcttaaggccagagggctgctgtcatgatttactgtccgt | |
| | cgtgtggacatgttgctcacacccgtcgcgcacatttcatggacgatggcaccaagata | |
| | atgattgcacagtgccggaatatttattgctctgcgacatttgaagcgagtgaaagcttttt | |
| | ctctgacagtaaagattcaggaatggaatacatttcaggcaaacagagataccgcgatt | |
| | cactgacgtcagcctcctgcggtatgaaacgcccgaaaagaatgcttgttaccggatat | |
| | tgttgtcggagatgtaaaggccttgcactgtcaagaacatcgcggcgtctgtctcagga | |
| | agtcaccgagcgtttttatgtgtgcacggatccgggctgtggtctggtgtttaaaacgctt | |
| | cagaccatcaaccgcttcattgtccgcccggtcacgccggacgaactggcagaacgc | |
| | ctgcatgaaaaacaggaactgccgccagtacggttaaaaacacaatcatattcgctgc | |
| | gtctggaatgagggctgccggttaacaccggccgtcgccgcacaccgtatttttattctt | |
| | cagcatgatgagaaagagataacgatggaaagcacagccttacagcaggcctttgac | |
| | acctgtcagaataacaaagcagcatggctgcaacgcaaaaatgagctggcagcggc | |
| | cgaacaggaatatctgcggcttctgtcaggagaaggcagaaacgtcagtcgcctgga | |
| | cgaattacgcaatattatcgaagtcagaaaatggcaggtgaatcaggccgccggtcgtt | |
| | atattcgttcgcatgaagccgttcagcacatcagcatccgcgaccggctgaatgatttta | |
| | tgcagcagcacggcacagcactggcggccgcactggcaccggagctgatgggcta | |
| | cagtgagctgacggccattgcccgaaactgtgccatacagcgtgccacagatgccct | |
| | gcgtgaagcccttctgtcctggcttgcgaagggtgaaaaaattaattattccgcacagg | |
| | atagcgacattttaacgaccatcggattcaggcctgacgtggcttcggtggatgacagc | |
| | cgtgaaaaattcacccctgcgcagaacatgattttttcgcgtaaaagtgcgcaactggc | |
| | atcacgtcagtcagtgtaaaattccccgaaaatccgcccgtttttactgaaaaaagccat | |
| | gcatcgataaggtgcatggcttt | |
| pP4- | gcatgcgttttcctgcctcattttctgcaaaccgcgccattcccggcgcggtctgagcgt | pP4-c2 |
| pLtetO1- | gtcagtgcaactgcattaaaaccgccccgcaaagcgggcgggcgaggcggggaaa | |
| Large | gcaccgcgcgcaaaccgacaagttagttaattatttgtgtagtcaaagtgccttcagtac | |
| | atacctcgttaatacattggagcataatgaagaaaatctatggcctatggtCCAAAA | |
| | CTGTCTTTTTTGATGGCACTATCCTGAAAAATATGCA | |
| | AAAAATAGATTGATGTAAGGTGGTTCTTGTCAGTGT | |
| | CGCAAGATCCTTAAGAATTCGTGGCATGAGAGAGTT | |
| | AAAGGGCGTCCCTATCAGTGATAGAGATTGACATCC | |
| | CTATCAGTGATAGAGATACTGAGCACATCAGCAGGA | |
| | CGCACTGACCGAATTCGAAAGAGGAGAAAGGAGAT | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnnnnnnnnnnnnnnnnnnnnnnnnnnnnnnnnnnnnnnnnnnn | |
| | nnnnnnnntaaCCCGGGCTCGGTACCAAATTCCAGAAAA | |
| | GAGGCCTCCCGAAAGGGGGGCCTTTTTTCGTTTTGGT | |
| | CCGAGCTCTGGTAAATAGAAACGGAACTTTACATAT | |
| | TGAATACCGGAGATATAATTCGTTTATGCCACATAA | |
| | TCATCAATACATCCGTAACCCGCCCGAAACGGAAAA | |
| | ACTGAAGAAAAACCCCATTATCTTAGCCTAAAAAAA | |
| | CCTTCTCTTTGGAACTTTCAGTAATACGCTTAACTGC | |
| | TCATTGCTATATTGAAGTACGGATTAGAAGCCGCCG | |
| | AGCGGGTGACAGCCCTCCGAAGGAAGACTCTCCTCC | |
| | GTGCGTCCTCGTCTTCACCGGTCGCGTTCCTGAAACG | |
| | CAGATGTGCCTCGCGCCGCACTGCTCCGAACAATAA | |
| | AGATTCTACAATACTAGCTTTTATGGTTATGAAGAG | |
| | GAAAAATTGGCAGTAACCTGGCCCCACAAACCTTCA | |
| | AATGAACGAATCAAATTAACAACCATAGGATGATAA | |
| | TGCGATTAGTTTTTTAGCCTTATTTCTGGGGTAATTA | |
| | ATCAGCGAAGCGATGATTTTTGATCTATTAACAGAT | |
| | ATATAAATGCAAAAACTGCATAACCACTTTAACTAA | |
| | TACTTTCAACATTTTCGGTTTGTATTACTTCTTATTCA | |
| | AATGTAATAAAAGTATCAACAAAAAATTGTTAATAT | |
| | ACCTCTATACTTTActagcataaccccttggggcctctaaacgggtcttga | |
| | ggggttttttgagacaaacaaaagaatggaatcaaagttaaTGCTAGCAGCc | |
| | GCCGctgcaggcatgcaagcttgcggccgcgtcgtgactgggaaaaccctggcg | |
| | actagtcttggactcctgttgatagatccagtaatgacctcagaactccatctggatttgtt | |
| | cagaacgctcggttgccgccgggcgttttttattggtgagaatccaggggtccccaata | |
| | attacgatttaaatttgtgtctcaaaatctctgatgttacattgcacaagataaaaatatatc | |
| | atcatgaacaataaaactgtctgcttacataaacagtaatacaaggggtgttatgagcca | |
| | tattcagcgtgaaacgagctgtagccgtccgcgtctgaacagcaacatggatgcggat | |
| | ctgtatggctataaatgggcgcgtgataacgtgggtcagagcggcgcgaccatttatc | |
| | gtctgtatggcaaaccggatgcgccggaactgtttctgaaacatggcaaaggcagcgt | |
| | ggcgaacgatgtgaccgatgaaatggtgcgtctgaactggctgaccgaatttatgccg | |
| | ctgccgaccattaaacattttattcgcaccccggatgatgcgtggctgctgaccaccgc | |
| | gattccgggcaaaaccgcgtttcaggtgctggaagaatatccggatagcggcgaaaa | |
| | cattgtggatgcgctggccgtgtttctgcgtcgtctgcatagcattccggtgtgcaactg | |
| | cccgtttaacagcgatcgtgtgtttcgtctggcccaggcgcagagccgtatgaacaac | |
| | ggcctggtggatgcgagcgattttgatgatgaacgtaacggctggccggtggaacag | |
| | gtgtggaaagaaatgcataaactgctgccgtttagcccggatagcgtggtgacccacg | |
| | gcgattttagcctggataacctgattttcgatgaaggcaaactgattggctgcattgatgt | |
| | gggccgtgtgggcattgcggatcgttatcaggatctggccattctgtggaactgcctgg | |
| | gcgaatttagcccgagcctgcaaaaacgtctgtttcagaaatatggcattgataatccgg | |
| | atatgaacaaactgcaatttcatctgatgctggatgaatttttctaacttggaagtaagaat | |
| | ggtgccgaaggccggactcaaacatcaaaataagttaatgataaaaaacaaataataa | |
| | aacacaacaatgaaatatgcccccttttgtgcccccactgtttttctgaccaatctattttca | |
| | gcccatcaataaatcggaaagttaaatcatttttaatcagtaagtttggatccgtagctcg | |
| | gatccaaaccagtgcatcttttatccacataaaaaattttttttcgaaagaactgttcacact | |
| | gttcacctttctgttttctccttttatttcagagtgataggtggtgaataatgggtgaagggt | |
| | gaacattcgattcttcacctccggcattctgccgatgtgactcataccggtgattaatcct | |
| | ccgcactgaaatcactcaggaagaaaaaagttttttttgatttgattgttcacactgttcac | |
| | ctttcgtttttctcttttaatttcagtgtgataacgggtgaatatacggtgaagggtgaacag | |
| | tggattgttcaccttcgggggatatcgggataaaaaaagaccggcagatgccggtcag | |
| | gtgggtcaggctgttgtagggtcgtcacattttggcagccagtcgccgtagctttcctctt | |
| | tcagcgtcaggttggtctgtatcccctgtttggtatggcgtttctcgtaattcagtccgtatt | |
| | ccttcagcatcaccggcagccccagcccgaacattttcagactgagtacattccggtag | |
| | ccgtttgcctccatgtaggccagataggcgtgatagaggtatttacggtaattgcgcgg | |
| | gatgatactggcgttccccatatacatgccgctggtctgcggcagggtttccagatagc | |
| | cgataaaatcaaacgtcgggtcggcatcccgtttgatgttcagtgcctcgtctgagttct | |
| | gctgggactgaagcagtgaccgggcgagcatcgggtcgctgaacttctgcatcaggt | |
| | gacgcacgatgaccgccagctcgcgggtgattttgtccttaagctgcgggtcgcgctc | |
| | ctgcggggctatctgttccgggaagtgaataatcacccgtcggcgtgacacgccgcc | |
| | gctgcggtcggtgaagcgcatcgggttattgttcacggccagaatcaccgccgggatg | |
| | tgcgtggagtacgcatcccggtatttcgggtcaacggacaccgcatcgccgccggtg | |
| | atggccttgagtccggcaccgtcgccgctccatttttcctggtccggcaggcgtatcagt | |
| | gagaagccagttaacgcggcacgttcacgcggggattccagcgtctcgatggtggcc | |
| | gacgtggcgttatcctccccggccagcagggtggctatttcggccatgatacttttgcc | |
| | gctgccgccgggaccggtcacctccagaaagagctgccagtcgtagcggtttgccag | |
| | caccataaacagtgcagccagaatcacgtcgcgtttttccgcacggccaccggcggc | |
| | acggtcaagccagcgccagaaggcgggggcgtgggtttccagcgtttcaccgtccac | |
| | cggcggggtgaaatccacatcgcacagggtgcgcatccagtgtgacggactgtgcg | |
| | ggtggaacgtgccgttctgcgtgtcgagcacgccgttacgaaagccaatcaggcggc | |
| | gggagggggcttcctgctgcggaataatcagcttcagggtatccaccacggaggcca | |
| | ccttcccggaggagaacggcgcacgcagacgctgaaacagcccggccacatcccg | |
| | ggcaaagtcctgtggcggcagcaccttccagacaccattttcatagcgggacagaag | |
| | ctggccgttggcatcgaccgcgagcgcctcgccgtaatgctcatagatacgcatggcc | |
| | ttttcgctggtactcatggcggaaaactccgcttcgctcatggtgtcgaacgggctttca | |
| | gccggtggccggatggcatcgtaaatggccttacgggtggcctccccgccgtactgc | |
| | gtgaaggcatcattccagtcaccgaagaccggcggcagggcaacaacaccttcacac | |
| | gcatctgcggctgcggcggcttttttctggccgtcaccactgaggtcacggtcagcgg | |
| | caaggacaatctgacaggcggggtgcttctgccgggcaaggctggccagagaaagg | |
| | aggttcacggaagaaagcgccaccatcaccgtttcaccggtcaggtgatgtacggtaa | |
| | gtgcggtcgcgtatccctccgctatccacagacgttttccggcctgattctgtccttcaag | |
| | ggtgtgacaggtgcccctgacctgtccgcctttcagggtgcgcttacggccgtcagca | |
| | ctgattaactgaaggttaaccagttcgccgctgtcgtcatacagtggcaccacaaggtc | |
| | accggcgcgccagctcacgccaccggctctgtgtgtgccggtcagcatccggcattc | |
| | ccggccgggaaagcccttgcgggtcaggtaggcgttaccggttccggtacgggttttc | |
| | gccatcagggtttgtgccagtgcggcggcgttcttccgggcagcgtctgtttcatcaac | |
| | ggcggcggtcgtcactgccgggtcagccggtggcaggctgccggtcacggcagcc | |
| | acctttgcggccgcgtcggacggggaaacaccaaaaaccttttcaaccagtttcaggc | |
| | cgtcaccggcaccacactgattgcagtaccaggtgccgcgcccctccctgtcatcaaa | |
| | acggaagcggtcactcccgccacagaccggacagggctgatgacggttcttcagcac | |
| | ctgaatccccagcgccgggagaatacgcggccagtggccgagcgcatggctgacg | |
| | gtggcggttacgttcattttcatggtgttgttctccttcagtgcagtaccggcgcttttatgt | |
| | gacgggcacagagttcatccatcacaaccagcccgagaaaggacagcgacggcgc | |
| | ggccttcagggggccggattccattaaatcttccagcagggcacaggctatctgacgc | |
| | cctttttcctcaccgtgctggcgcagataaaagccttccagctcagcggcgatggccgc | |
| | ctccagtgactcaagggtgagatgcgggtagcggtgctgacgttcgcacacggtcag | |
| | ccaggcacaggcgacagcgcgacggtaaagggcagcgcgtaagacgggcggtaa | |
| | gggtgttttcatttgcttttctccctgtgacagatgactgcattccgtgccggttgcattaac | |
| | tgataaggcatatctgcgtctcctgaagacgtgcgtatccctgcgcgaatacgcacattt | |
| | aatttttcgggggtcgttttttaattacagataattgcggtaactgttatccggggtggtttc | |
| | cgggtcaggctccgtgcggggaatttcccgccattcccgcgccaccggtgctgcccg | |
| | gctgaccggaacagtgtcctgcgggtaaatatccagatatttttcccgccatttctgtaatt | |
| | ccgggtctccggccatttctttcagtaccgcatgccggtttacggggctgcgtttaaaca | |
| | ggtcaggacggtcacaggtaaattcccgcagaaaacgccccagcgggatgtctgtgg | |
| | tgcgtccgtcagcgaggatacgcacaaggatactgaatttacggcggtacgggttcca | |
| | gacaatgtccgggcagcggtacggcatttcccacggaataccgtcttccagaatgccg | |
| | accacggccacatcgggaaaaccggcagaacggtaaatctcaccgggctggggaa | |
| | aatcaaacatgcgtcctgtctccccggtctttctgctgggcgagaaaatcgcggcacag | |
| | gcctttggctttcagctcattcagcacaaaatcaatatcttcattcaggtagctgaaaatat | |
| | gtggaatgtagagctgatgcaggccggagagttcacggtgaatcaaatcacccccaa | |
| | caaaccgggatacggcgctggcgcggttgagcttatggtaagcctcaatgctgaggtg | |
| | ttcacgggcgtcatgacgcgctgagacggtctgaggggcttttttattacgcacgggac | |
| | acctccaccaccggcagacgggcagcaagggagagcacatagtcacggacaaggg | |
| | aacggcgggcactgcgttcatcaccggcgacggtgcgaagcatacagatacgggg | |
| | tgacggtctgcgcgacggacagccgcaaacacaaagacaaattcagggtgtgaggg | |
| | ggtaagggttgtagccatgatggcagcctcctgtgaatagcaaataacgctatcgccg | |
| | gagttctcacgctcgatggcgatagcccagacgggggtgagaataccggcttcacag | |
| | gataccggccagcccggaggctgccccgcctgagctaccattgactctgcggcataa | |
| | tgagcggacgcgggcaggatgcacggaatgccatctgcacgactgaccacacacca | |
| | caccataatctggcgctctgtggcattgattgcgacacaaaaaaagacgcgtggcgcg | |
| | tcatatgtcgcctgtgaattgctcgggttctcacgcccggctgccgattttgcggcaggc | |
| | gaaaaactatatccgcaaatgccggaaaaaggcaagccagaaaaagggagtttttgc | |
| | agagcgggcatcatcatgcgtcgtacccccgtttgcgtccggcaatgcgtccggccat | |
| | ccatgcggtgacttcagagtgcagccaggccacatttttaccgccaagactcacctgc | |
| | ggcggaaattcccccttacggatgagttcgtagatggtcgagcgtgacaggccgcac | |
| | aggtgcatcacttccggcacacgtaaaaaacgctcctgcgtgatgtccggcagcggc | |
| | atcagtggcgtcactggggcgggagacggggaagaaaaaacagcttgcatcgggct | |
| | acctcgttaatgtccatacagcaccggataagtccgtccggcttcgggtagcgctttattt | |
| | tgtgaatattttcagcagacgcaacaggggggatttgttcaggctgtcttacaatggctgt | |
| | gtgttttttgttcatctccacttaaagtcatttaaagccacttaaagcaatttgtaatttttatag | |
| | tgaaatacaaatcgtttcttcttattcattcccggcgaattaataaaaacaaacagtagtaa | |
| | acagcacaaaaagcccatcaacgggtgaacagtggtgaacagacggtgaacagtca | |
| | ttactgcgattgttcaccctttaacttactgtattacttatcttttttattaaggtgaacagagg | |
| | tgaacagtaaaatataaaaaaacaaacagtaagccggtttttcctgcgaccttttcctgg | |
| | cttgccggtctgaggatgagtctcctgtgtcagggctggcacatctgcaatgcgtcgtgt | |
| | tgttgtccggtgtacgtcacaattttcttaacctgaagtgacgaggagccggaaaatgtc | |
| | tgaccacactatccctgaatatctgcaacccgcactggcacaactggaaaaggccaga | |
| | gccgcccatcttgagaacgcccgcctgatggatgagaccgtcacggccattgaacgg | |
| | gcagagcaggaaaaaaatgcgctggcgcaggccgacggaaacgacgctgacgact | |
| | ggcgcacggcctttcgtgcagccggtggtgtcctgagcgacgagctgaaacagcgc | |
| | cacattgagcgcgtggcacgccgggagctggtacaggaatatgacaatctggccgtg | |
| | gtgctgaatttcgaacgtgaacgcctgaaaggggcgtgtgacagcacggccaccgcc | |
| | taccggaaggcacatcatcaccttctgagtctgtatgcagagcatgagctggaacacg | |
| | ccctgaatgaaacctgtgaggcgcttgtccgggcaatgcatctgagcattctggtacag | |
| | gaaaatccgctcgccaacaccaccggccatcagggctacgtcgcaccggaaaaggc | |
| | tgtcatgcagcaggtgaaatcatcgctggaacagaaaattaaacagatgcaaatcagc | |
| | ctcaccggcgagccggttctccggctgaccggactgtcagcggcaacactcccgcac | |
| | atggattatgaggtggcaggcacaccggcacagcgcaaggtgtggcaggacaaaat | |
| | agaccagcagggagcagagcttaaggccagagggctgctgtcatgatttactgtccgt | |
| | cgtgtggacatgttgctcacacccgtcgcgcacatttcatggacgatggcaccaagata | |
| | atgattgcacagtgccggaatatttattgctctgcgacatttgaagcgagtgaaagcttttt | |
| | ctctgacagtaaagattcaggaatggaatacatttcaggcaaacagagataccgcgatt | |
| | cactgacgtcagcctcctgcggtatgaaacgcccgaaaagaatgcttgttaccggatat | |
| | tgttgtcggagatgtaaaggccttgcactgtcaagaacatcgcggcgtctgtctcagga | |
| | agtcaccgagcgtttttatgtgtgcacggatccgggctgtggtctggtgtttaaaacgctt | |
| | cagaccatcaaccgcttcattgtccgcccggtcacgccggacgaactggcagaacgc | |
| | ctgcatgaaaaacaggaactgccgccagtacggttaaaaacacaatcatattcgctgc | |
| | gtctggaatgagggctgccggttaacaccggccgtcgccgcacaccgtatttttattctt | |
| | cagcatgatgagaaagagataacgatggaaagcacagccttacagcaggcctttgac | |
| | acctgtcagaataacaaagcagcatggctgcaacgcaaaaatgagctggcagcggc | |
| | cgaacaggaatatctgcggcttctgtcaggagaaggcagaaacgtcagtcgcctgga | |
| | cgaattacgcaatattatcgaagtcagaaaatggcaggtgaatcaggccgccggtcgtt | |
| | atattcgttcgcatgaagccgttcagcacatcagcatccgcgaccggctgaatgatttta | |
| | tgcagcagcacggcacagcactggcggccgcactggcaccggagctgatgggcta | |
| | cagtgagctgacggccattgcccgaaactgtgccatacagcgtgccacagatgccct | |
| | gcgtgaagcccttctgtcctggcttgcgaagggtgaaaaaattaattattccgcacagg | |
| | atagcgacattttaacgaccatcggattcaggcctgacgtggcttcggtggatgacagc | |
| | cgtgaaaaattcacccctgcgcagaacatgattttttcgcgtaaaagtgcgcaactggc | |
| | atcacgtcagtcagtgtaaaattccccgaaaatccgcccgtttttactgaaaaaagccat | |
| | gcatcgataaggtgcatggcttt | |

### Preparation and Quantification of Helper-Free Phage-Like Particles

The particles were produced and quantified as described by Ababi et al. 2022, outlined in Methods Section 4.3 and Section 4.9. The biological replicates were mixed and concentrated approximately 100× via the 100 kDa Vivaspin 20 ultrafiltration falcon systems, according to the manufacturer's instructions. In between experiments, raw lysates and final stocks were stored in the fridge at 4-8° C. To avoid stability errors when calculating accurate MOIs, the quantification of particles was performed in triplicate technical repeats and a day prior to each transduction assay.

### Transduction Assay

4 mL of Luria Bertani (LB) media were inoculated with E. coli EMG2 from a glycerol stock, and incubated overnight at 37° C., 200 RPM. Next day, the overnight culture was refreshed in 10 mL LB supplemented with 2 mM CaCl₂) and grown until OD600=0.3-0.4, shaking at 37° C. The protocol continues either on a plate reader or in a culture tube.

For the plate reader experiment, the concentrated stocks were adjusted to a titer of 2×10⁹ PFU/mL in LB and 100 μL each was added to a 96 well plate compatible with the Tecan plate reader. The tecan programme was set, and the instrument was heated in advance to 37° C. At OD600=0.3-0.4, the EMG2 culture was diluted down to an OD600≈0.2 in LB (+2 mM CaCl₂)) just before mixing with the particles. 100 μL of OD600 adjusted culture was then transferred via a 12 channel pipette to the corresponding wells. OD600 measurements were recorded every 5 min. The expression and secretion of the proteins were monitored at 3 hrs and 6 hrs time points.

Where transduction was done in a tube instead of the 96 well plate, the culture tubes containing the cells and particles mixture (MOI of 0.1 or 10) were incubated for 3 hrs, 6 hrs and overnight at 37° C., shaking with aeration.

At corresponding time points, respective transduced cultures, were transferred to a 1.5 mL Eppendorf tube and pelleted immediately (7 min; 13,000 RPM). The respective supernatants were concentrated individually down to 25 μL via Vivaspin 500 with 10 kDa cut-off, at 4° C., and according to the manufacturer's instructions. Each concentrated supernatant was then mixed with 25 μL Gel Loading Buffer (100 mM Tris-Cl (pH 6.8), 4% (W/V) SDS, 0.2% (w/v) bromophenol blue, 20% (v/v) glycerol, 200 mM dithiothreitol) and boiled for 10 min at 95° C. The possibly secreted protein was now denatured, and hence safe to store at −20° C., before further analysis (e.g. SDS-PAGE, Western blot).

### Cargo Stability

The payload after the production of particles was delivered to EMG2 bacteria via the transduction protocol adjusted from Ababi et al. 2022, Methods Section 4.10. Here the target strain was EMG2 instead of DH5α-Z1, and hence spectinomycin was not added at respective steps. The successfully transduced colonies were isolated and grown overnight in 50 μg·mL⁻¹ kanamycin. The respective phasmids were miniprepped using the QIAprep Spin Miniprep kit and sent for Sanger sequencing together with individual primers flanking the cargo region:
5′-catacctcgttaatacattggagc-3′
5′-cagggttttccttaactttgattccattcttttgtttg-3′

### Example 2—Comparison of Different Promoters for Expression and Stability of Cargo Genes

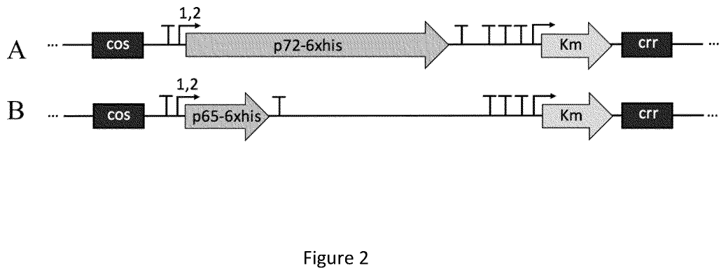

We engineered four P4 phasmid constructs containing cargo: the phasmids encoded a secretory protein of 21 kDa (detoxified ADP-ribosyltranferase subunit S1; dubbed p65) and, separately, a secretory protein of 73 kDa (detoxified bifunctional hemolysin/adenylate cyclase; dubbed p72), both controlled by two promoters: LacIq and pLtetO-1 (FIG. 2). Both secretory proteins were tagged with a 6×his at the 3′ end to facilitate detection by western blot via anti-his antibodies. The anti-5×his detected tagged proteins, while the 6×his did not (coomassie blue stained gel vs western blot; data not included), potentially due to the inwards folding of the first histidine linker.

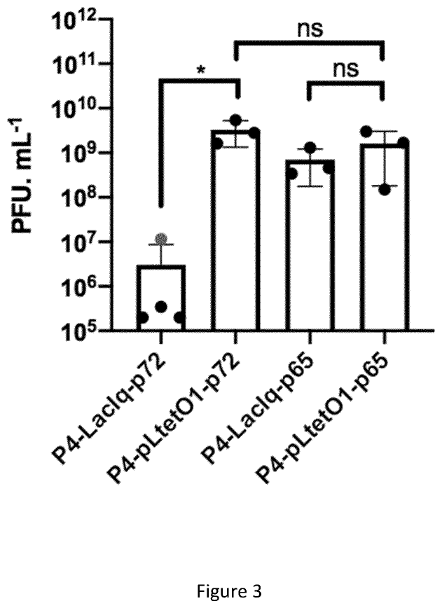

Small lots of phage-like particles were prepared as optimized in Ababi et al. 2022. Expressing p72 constitutively during the production of particles in E. coli C-5545Δcos-Marrionette, resulted in significantly fewer particles compared to when the respective recombinant protein expression was repressed (FIG. 3). This could be due to the production cells being under significant stress to both express the p72 (molecular weight 73 kDa) protein and produce particles. This effect was not observed for the smaller secretory protein, p65 (molecular weight 21 kDa), where the difference in yield between the two expression mechanisms was insignificant.

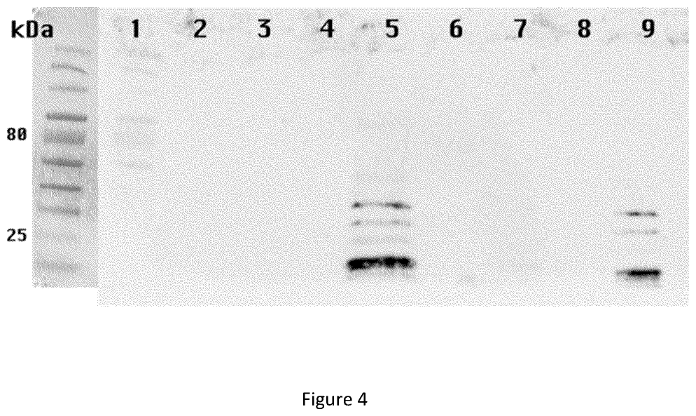

The biological production lots were then mixed and concentrated approximately 100× by Vivaspin 20 ultrafiltration, with 100 kDa cut-off. We reached ˜5×10⁸ PFU/mL for P4-LacIq-p72, ˜1.3×10¹¹ PFU/mL for P4-pLtetO1-p72, ˜3.7×10¹⁰ PFU/mL for P4-LacIq-p65, and ˜5×10¹⁰ PFU/mL for P4-LacIq-p65. At these concentrations, we could only use dilutions of the later three constructs to transduce into 100 μL E. coli EMG2 cells at an equal MOI of 10 (1:1 volume). The protein secretion was identified in twice concentrated supernatant by SDS-PAGE and western blot after three and six hrs post-transduction (FIG. 4). Due to the limited number of particles, the expression of p72-LacIq encoding particles was evaluated separately.

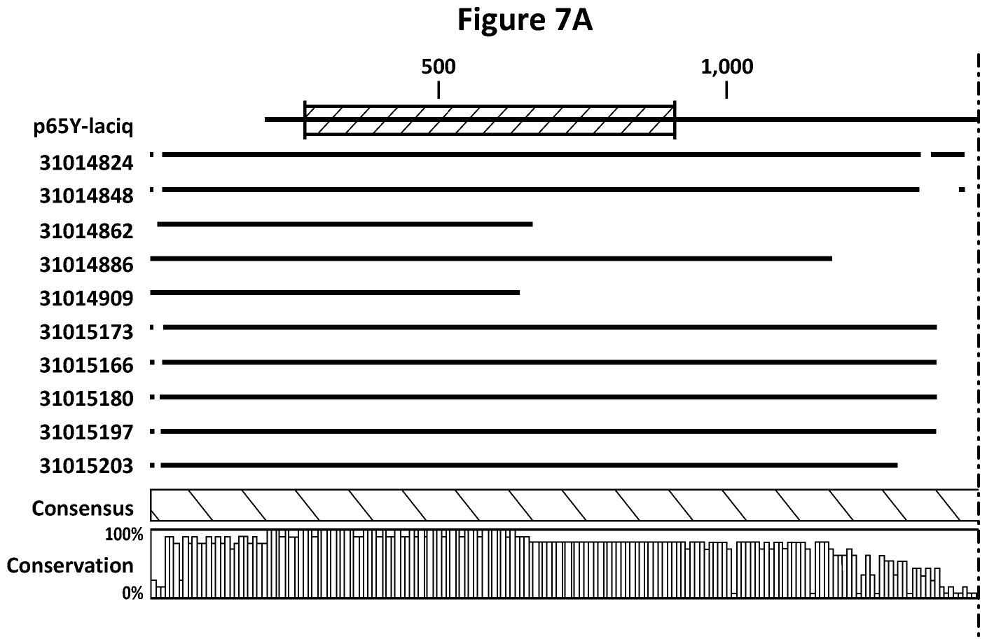

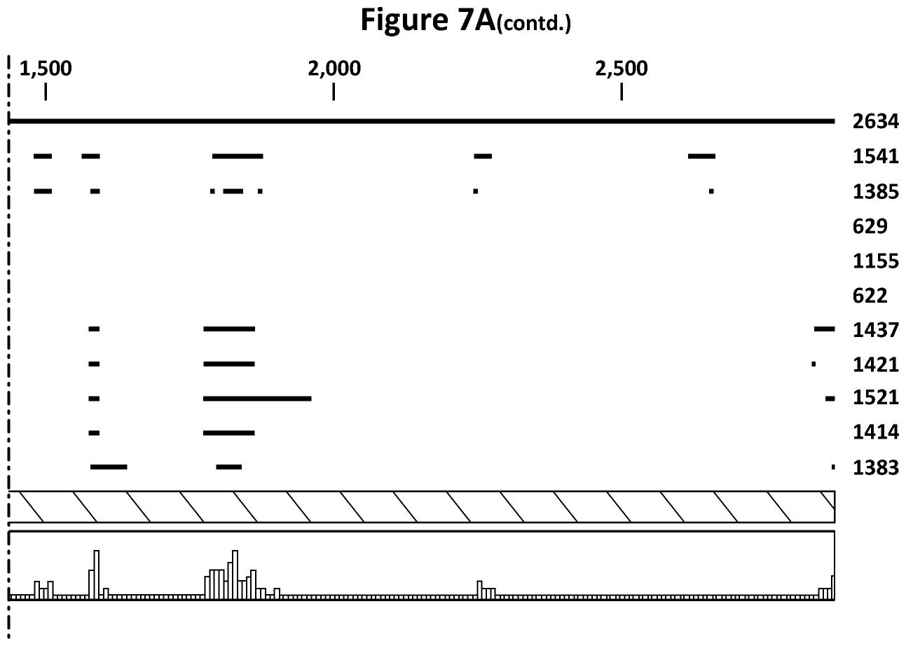

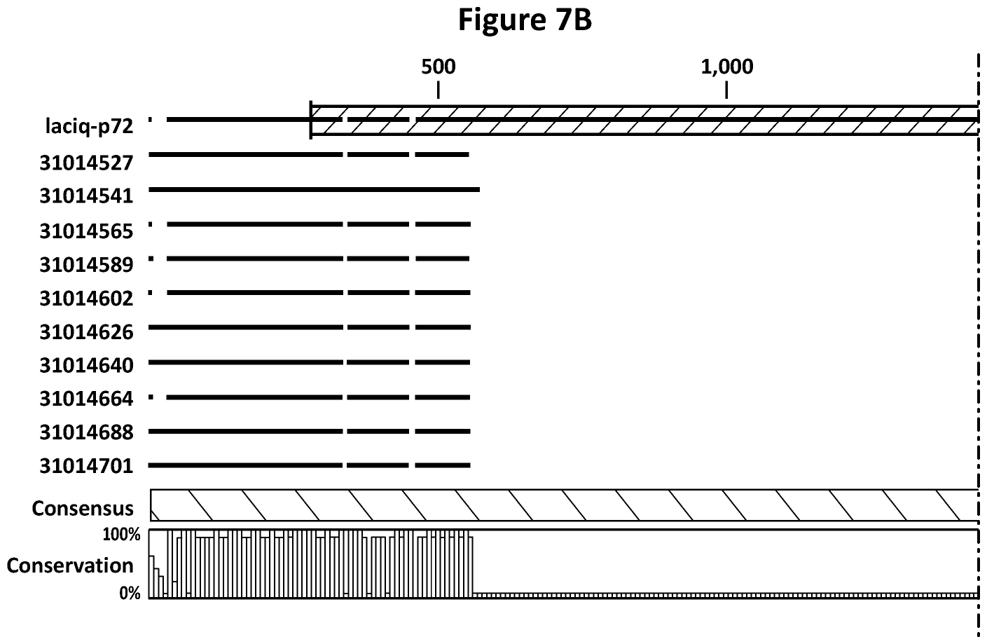

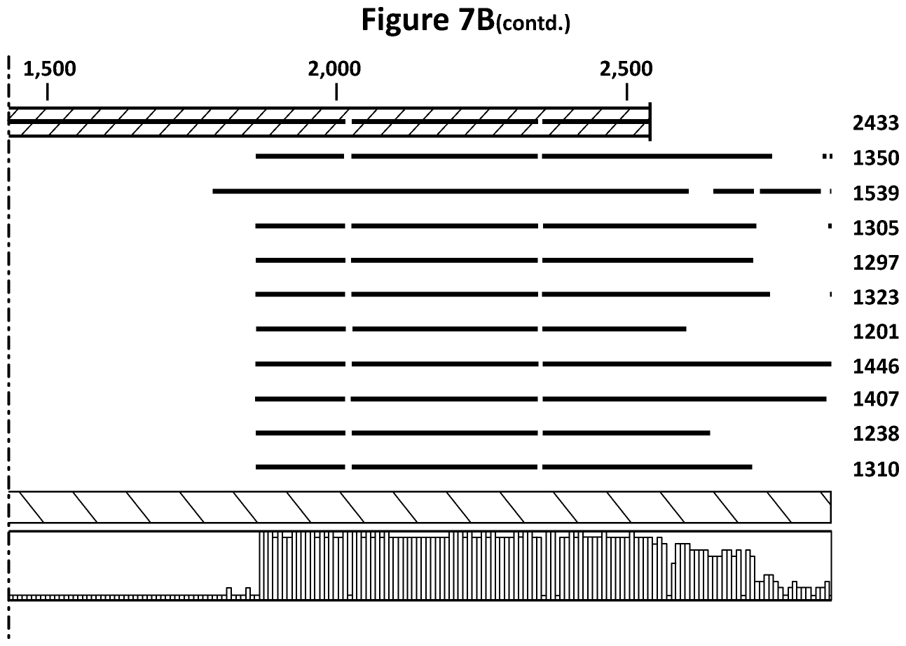

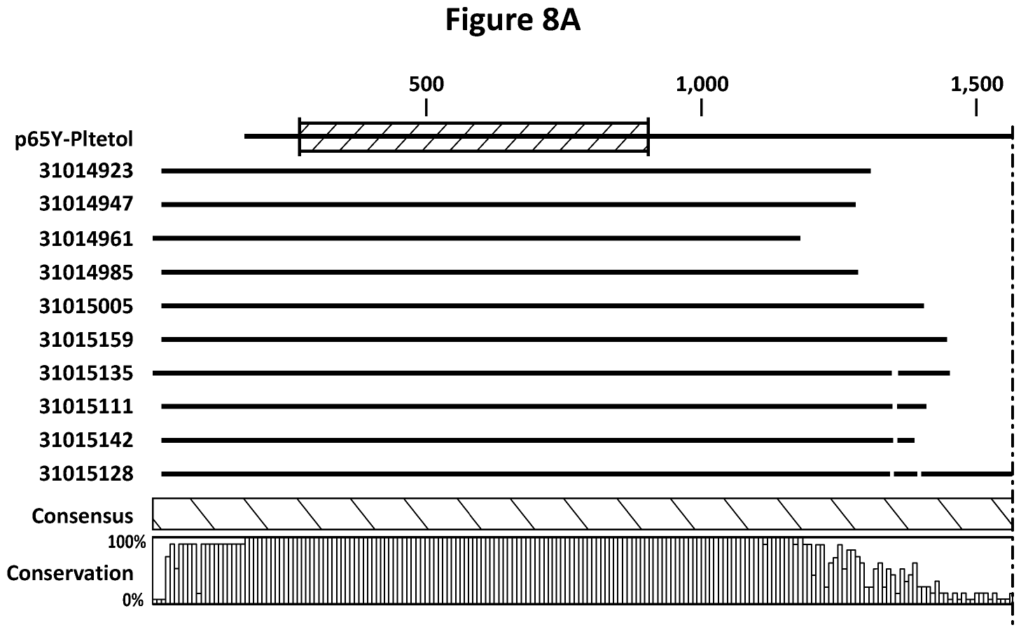

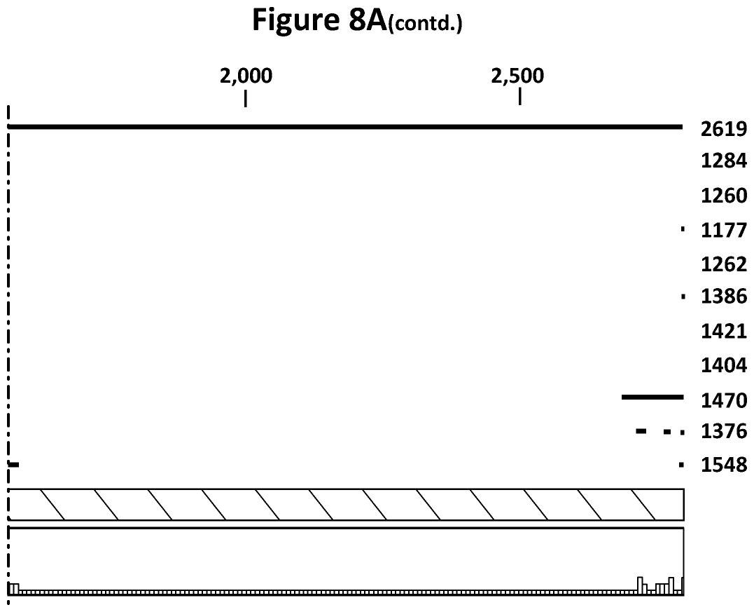

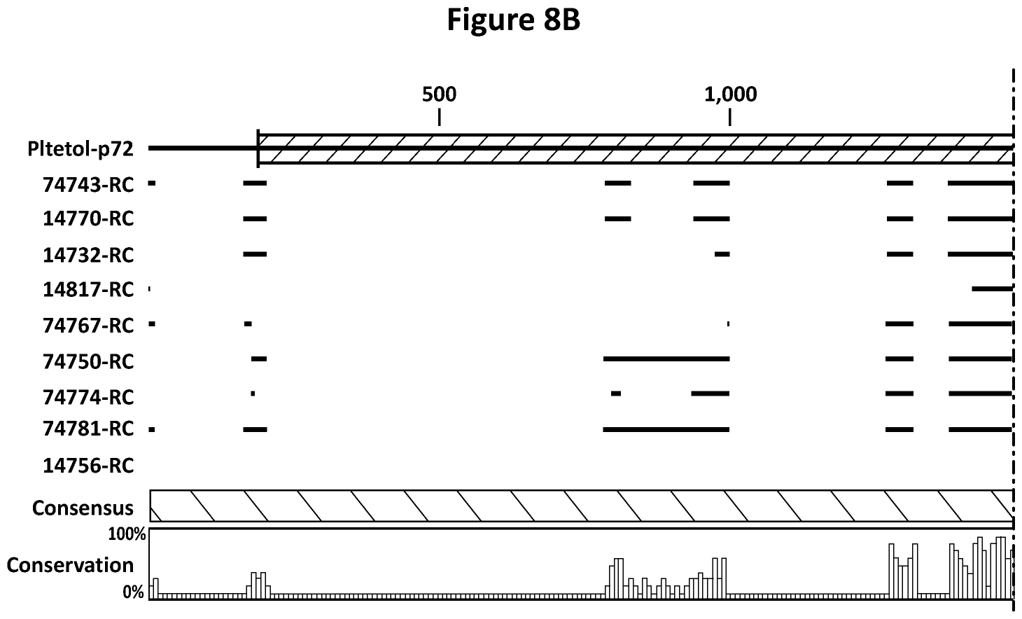

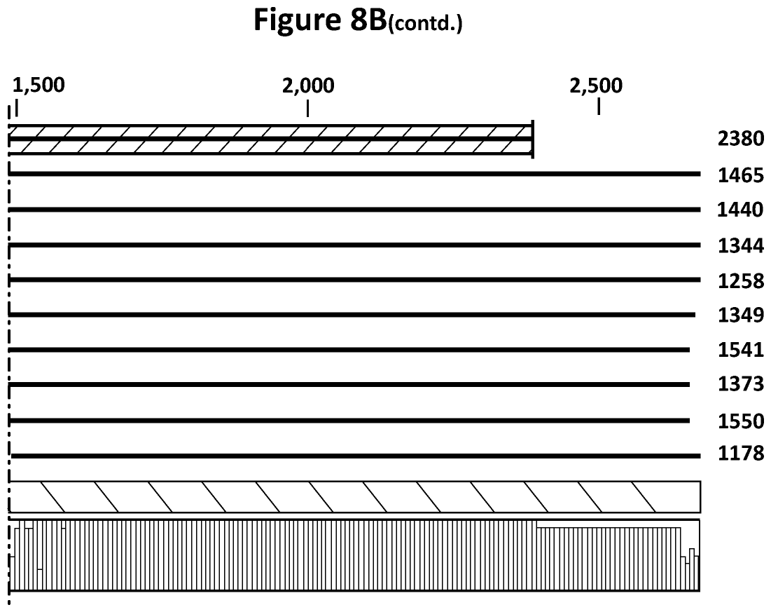

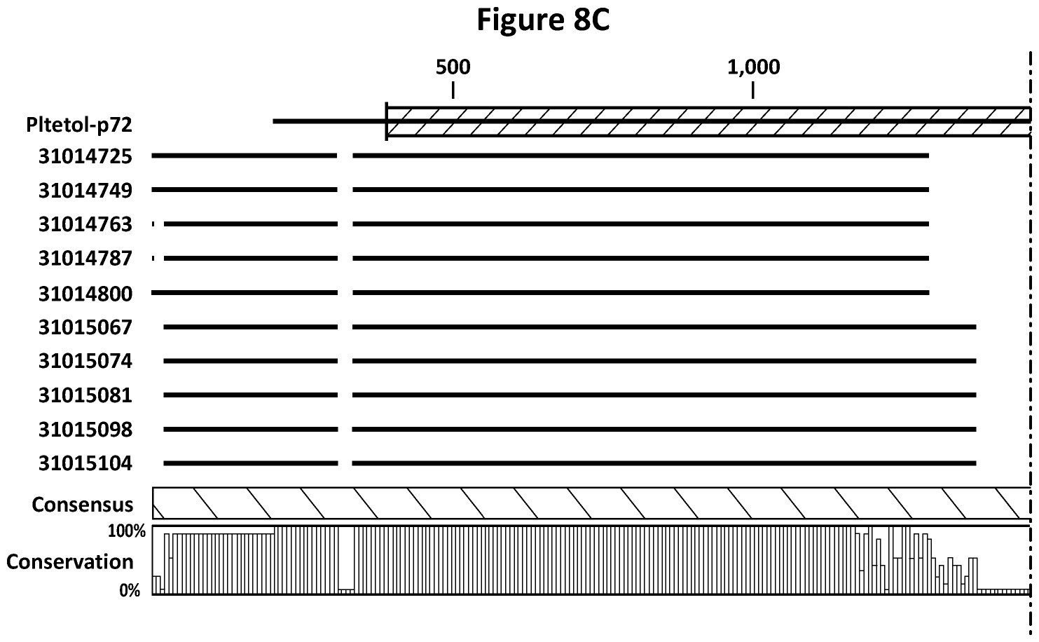

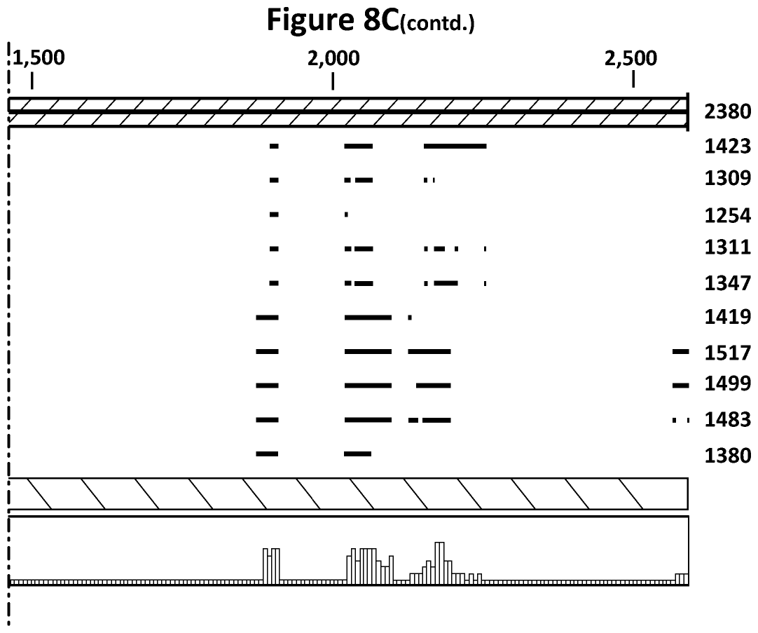

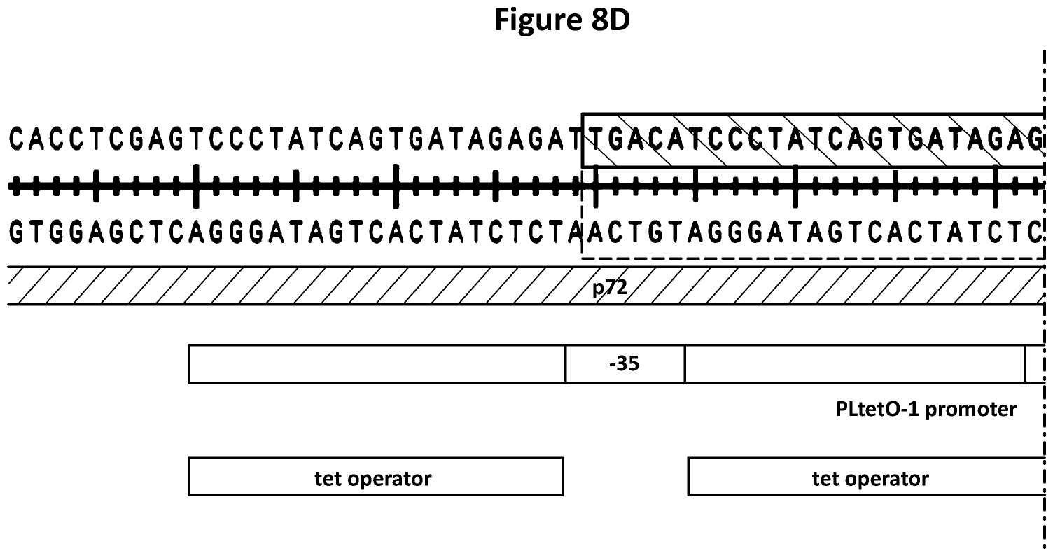

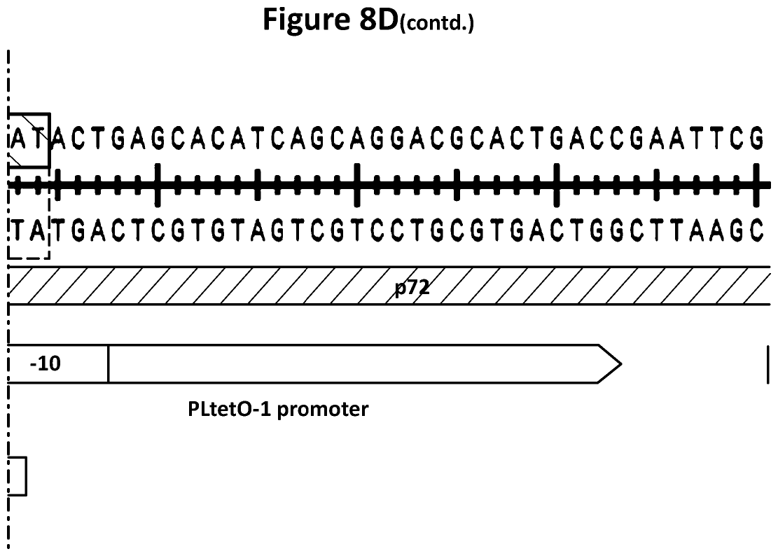

Out of the three constructs delivered (p72-pLtetO1, p65-LacIq and p65-pLtetO1), only the small protein, p65, was detected in the supernatant after 6 hrs post-transduction. The p65 protein was detected both when controlled by LacIq and pLtetO-1. The cargo sequence was confirmed by a separate transduction into EMG2 cells and overnight selection onto LBA plates with kanamycin. 10 colonies were isolated and grown overnight in kanamycin. Their respective transduced phasmids were miniprepped next day and sent for Sanger sequencing in the cargo region (FIG. 7A, FIG. 8A). Out of the 10 colonies investigated each, the p65-pLtetO1 resulted in 10/10 perfect match, compared to p65-LacIq that resulted in 8/10 perfect match and 2/10 cases with mutated cargo.

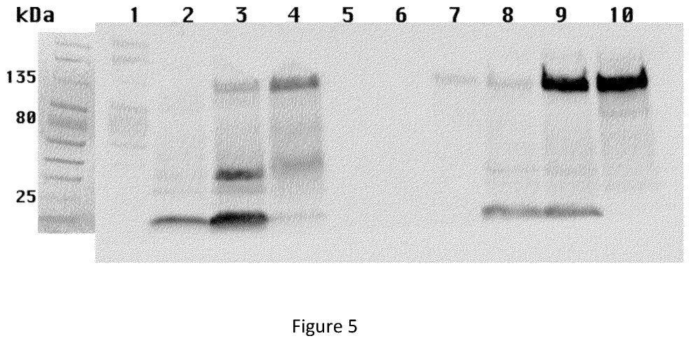

To further investigate the ability of engineered P4-LacIq/pLtetO1-p72 particles to express and secrete large proteins after transduction, we transduced one mL E. coli EMG2 at different MOIs (0.1, 10), and monitored the secretion in 20 times concentrated supernatant after 3 hr, 6 hr and overnight incubation at 37° C. (FIG. 5).

The p72 was detected in the 20× concentrated supernatant after transduction. The expression was detected more strongly when controlled by the LacIq promoter compared to the pLtetO-1 promoter at the same MOI equal to 0.1. This being said, at MOI of 10, which was only possible to test with the pLtetO-1 controlled expression, the concentration of protein is significantly improved after 6 hr and overnight.

The stability of the p72 cargo region was assessed by Sanger sequencing as mentioned above for the p65 cargo region (FIG. 7 B, FIG. 8 BC). Out of the 10 successful transductants screened for each, 10/10 p72-LacIq cargos contained a fragment deletion in the p72 region between 82 aa-517 aa, explaining the lower sized bands on the western blot. This being said, the full size p72 product is also detected on the western blot, indicating the presence of a mixed population of phage-like particles coding for full and shorter p72 variants. For the p72-pLtetO1 cargos, 10/10 contained instead a deletion between the −35 and −10 region of the pLtetO-1 promoter (FIG. 8 D), however with a 10/10 perfect match trace for the p72 sequence. This explained the reduced expression of the cargo at equal MOIs and the cleaner western blot profile compared to the LacIq promoter.

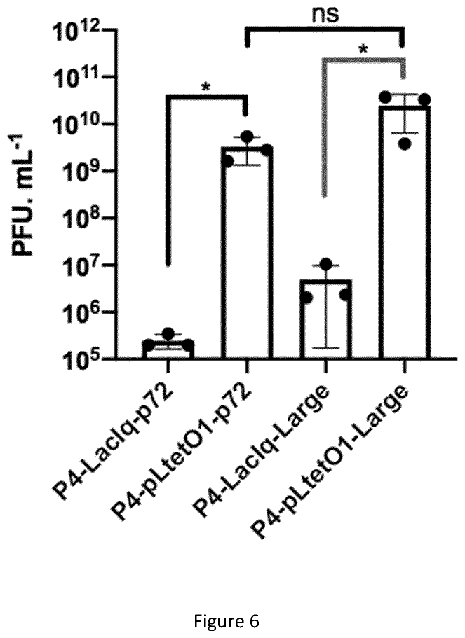

We wanted to check whether the constitutive expression of large constructs in the production strain, C-5545Δcos-Marrionette, was indeed inhibiting the production of particles universally, as opposed to being a random event due to the random deletion/mutations or due to the 2 bps difference in size. For this, we designed and ordered from Genewiz two new P4 phasmids, both at 11,624 bps, encoding for a large secretory system of ˜61 kDa, controlled by LacIq and pLtetO-1 promoters, and produced particles as before (FIG. 6). Approximately 4 logs difference was detected once again between the two expression mechanisms during the production of particles encoding the new large secretory system, confirming the universal inhibitory effect of large secretory proteins on the particles' yield.

### CONCLUSION

We produce phage-like particles encoding for two B. pertussis antigens controlled by two sets of promoters: LacIq and pLtetO-1. The antigens are delivered via phage-like particles to E. coli where they are secreted and detected in the supernatant by SDS-PAGE and western blot analysis. The production of particles was inhibited by the constitutive expression of the larger B. pertussis genetic toxoid, p72, while the co-expression of the smaller toxoid, p65, made no statistical difference in the particles' yield. Both cargos were found more stable when their expression was repressed during the production of particles, and the p65 secreted faster than the p72 proteins. Information presented here could aid in the optimization of engineered phage designs to harness the use of commensals/microbiota to induce immunity to vaccine antigens, and or encourage future use of commensal/microbiota based therapeutics using the P4-based system for delivery.

Repressing the expression of the secretory proteins during the production of helper-free engineered phage-like particles improved the particles' yield and cargo stability. Testing a smaller sized cargo also proved beneficial, together signalling that the cargo-induced burden on the production strain could create a selection pressure for randomly mutated variants. For this reason, it is advantageous to repress the cargo expression during the production of particles.

### Example 3—Use of Promoters in Prime and Kill Strategies

P4 phasmids were constructed to test for differential expression of dual-action cargo using as vector backbone the P4 phasmid described in Tridgett et al. 2021. The combinations of promoters shown in Table 2 were used to drive expression of the lytic cassette and the antigen PE.

Table 2. P4 phasmid designs for testing the functionality of the dual-action cargo after transduction into EMG2 cells.

TABLE 2
P4 phasmid designs for testing the functionality of the
dual-action cargo after transduction into EMG2 cells.

| | | | | | Lytic | |
|---|---|---|---|---|---|---|
| | | | Lytic | | cassette/ | |
| | | | cassette | Antigen | antigen | |
| | Lytic | | expression in | expression in | expression | Vector |
| | cassette | PE | production | production | in target | backbone/ |
| Construct | promoter | promoter | strain | strain | strain | Reference |
| pP4- | DiffJJ | NA | repressed | NA | constitutive | Tridgett et |
| DiffJJ- | | | (weak) | | | al. 2021 |
| Test | | | | | | |
| pP4- | DiffJJ | LacIq | repressed | constitutive | constitutive | pP4-DiffJJ- |
| DiffJJ- | | | (weak) | (strong) | | Test |
| LacIq | | | | | | |
| pP4- | DiffJJ | LacI | repressed | constitutive | constitutive | pP4-DiffJJ- |
| DiffJJ- | | | (weak) | (weak) | | LacIq |
| LacI | | | | | | |
| pP4- | pLtetO-1 | LacIq | repressed | constitutive | constitutive | pP4-DiffJJ- |
| pLtetO1- | | | (strong) | (strong) | | LacIq |
| LacIq | | | | | | |
| pP4- | pLtetO-1 | tac | repressed | repressed | constitutive | pP4- |
| pLtetO1- | | | (strong) | (strong) | | pLtetO1- |
| tac | | | | | | LacIq |

Particles were amplified using a lysate to infect the production cells, C-5545Δcos-Marionette grown anaerobically overnight, at an MOI ˜0.45. This resulted in about 10⁹ PFU/mL, titer.

Following transduction into E. coli EMG2 at a range of MOIs from 1 to 10 each, lysis was detected for all promoter sets from the lowest MOI tested (FIG. 10).

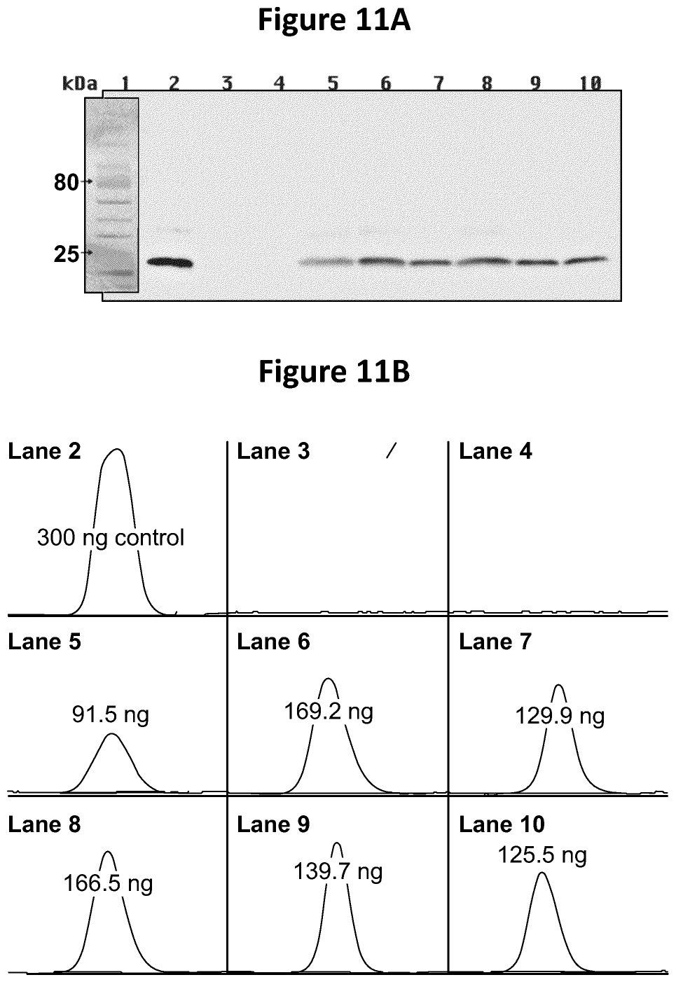

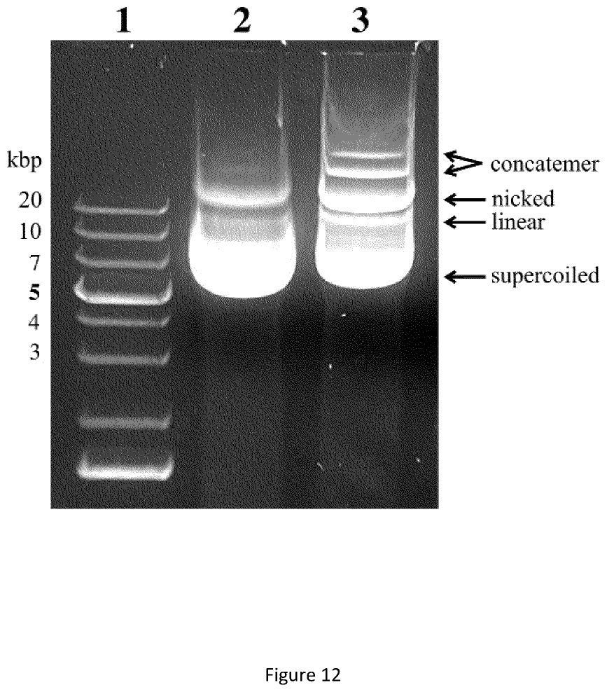

To examine whether the antigen is also expressed during the accumulation of lysins before cell lysis, samples of the post-transduction culture were collected at half-time and full-time lysis. Culture supernatants were collected and concentrated by ultrafiltration with 10 kDa cut-off. The antigen presence in the concentrated supernatants was evaluated by western blot and comparison with a PE control containing a known amount of protein (FIG. 11A). By running side by side with a PE control of known amount (300 ng), the amount of PE present in the supernatant was estimated using the ImageJ gel analysis tool (FIG. 11B).

In this study we demonstrated instead a novel antimicrobial approach based on phage-mediated delivery of custom cargo to both clear bacteria efficiently, and also to simultaneously produce a secondary therapeutic protein before lysis. For this, we engineered four sets of P4-like particles encapsulating cargo coding for a lytic cassette and the PE protein regulated by the following promoter pairs, respectively: DiffJJ-LacIq, DiffJJ-LacI, pLtetO1-LacIq and pLtetO1-tac and demonstrated that the dual-action cargo regulated by the later three, allowed the particles to behave as dual-action antimicrobials. Only the particles carrying cargo coding for the DiffJJ-LacIq promoter pair resulted in protein accumulation with no host lysis, suggesting there is a competition for resources in the target strain between the differentially expressed cargos. By balancing the strength of the promoter pairs as both weak or both strong variants, DiffJJ-LacI or pLtetO1-LacIq/tac, respectively, we were able to recover the activity of the lysis cassette during the simultaneous expression of PE. The particles encapsulating the cargo controlled by the DiffJJ-LacI and pLtetO1-LacIq promoter pairs also led post-transduction to an increased accumulation in the supernatant at half-time compared to full-time lysis, demonstrating without doubt the dual-action functionality of their cargo.

### Example 4—Modulating Cargo Capacity

This study challenged the ability of the P4 phage to increase its cargo capacity by concatenation into P2-sized capsids. To induce production of P2-sized capsids only, the sid gene on a P4 phasmid was fully deleted except the last 4 bps overlapping with the delta gene at the 3′ end. To this phasmid was added a cargo rendering it too large to be packed inside P4-sized capsids (pP4Δsid-large; 11,870 bps). As a negative control for concatenation, a P4 phasmid able to be packed into P4-sized capsids (pP4-c2; 11,627 bp) and induce preferential production of P4-sized capsids with only baseline level of P2-sized capsids was used.

Lysates generated using the pP4-c2 and pP4Δsid-large constructs were prepared. Particles encapsulating respective phasmids were transduced into DH5α-Z1 and selected the successfully transduced bacteria by plating onto agar supplemented with kanamycin. Colonies were picked next day, grown overnight, and the transduced phasmids were miniprepped. The sizes of the undigested transduced pP4-c2 and pP4Δsid-large phasmids were then measured by gel electrophoresis to check for concatenation (FIG. 9).

The transduced pP4Δsid-large phasmid showed two bands larger than the transduced pP4-c2 phasmid, confirming the concatenation effect inside the larger P2-like capsid. Other than the 2 larger bands, the pP4Δsid-large phasmid ran similarly on the 1% agarose gel, suggesting the concatenated pP4Δsid-large phasmid is likely to revert back to original size after being injected into the target cells. Importantly, the production of phage-like particles encapsulating larger cargo into P2-sized capsids resulted yet still in zero P2 helper contamination and hence remains safe for preclinical assays. The titer respective to the pP4Δsid-large production was however low at 10³ particles per mL and requires further optimization for practical applications.

1. An engineered bacteriophage comprising a polynucleotide encoding a heterologous protein under the control of a repressible promoter.

2. The engineered bacteriophage of claim 1 wherein the heterologous protein is a therapeutic or prophylactic agent.

3. The engineered bacteriophage of claim 1 wherein the heterologous protein is an antigen and/or immunomodulatory agent.

4. The engineered bacteriophage of claim 1 wherein the heterologous protein is capable of killing a bacterium.

5. The engineered bacteriophage of claim 3 wherein the antigen is a protein from a bacterium.

6. The engineered bacteriophage of claim 1, wherein the antigen is naturally present in a bacterium that is capable of being infected by the bacteriophage.

7. The engineered bacteriophage of claim 6 wherein the antigen is a Staphylococcus aureus, Streptococcus pneumoniae, Shigella, Pseudomonas aeruginosa, Cutibacterium (Propionibacterium), Acinetobacter, Neisseria meningitidis, E. coli, C. difficile, C. acnes (P. acnes), K. pneumoniae, Neisseria gonorrhoea, Fusobacterium nucleatum P. gingivalis or Mycobacterium avium paratuberculosis (MAP) antigen.

8. The engineered bacteriophage of claim 1 wherein expression of the antigen in a bacterial host cell leads to metabolic strain in a bacterial host cell.

9. The engineered bacteriophage of claim 1 wherein the heterologous protein has a molecular weight of at least 10 kDa, 20 kDa, 30 kDa, 40 kDa, 50 kDa, 60 kDa, 70 kDa, 75 kDa or 80 kDa.

10. The engineered bacteriophage of claim 1 wherein the promoter is a repressible promoter selected from the group consisting of pLtetO1promoter, pLtetO-1 promoter variants (W, S, E, R, JJ, P and II), tac promoter, pVanCC promoter, PhIF promoter, CymRC promoter, BetI promoter, Tlg promoter and 3B5C promoter.

11. A process for producing a bacteriophage preparation, wherein the process comprises producing the engineered bacteriophage of claim 1 under conditions where expression of the heterologous protein is repressed.

12. A process for producing an engineered bacteriophage comprising a polynucleotide containing a gene encoding a heterologous protein under the control of a repressible promoter, wherein the process comprises the steps of i) amplifying the engineered bacteriophage in bacteria in the presence of a repressor of the repressible promoter and ii) isolating the engineered bacteriophage.

13. An engineered bacteriophage genome polynucleotide comprising a bacteriophage packaging sequence and a gene encoding a heterologous protein under the control of a repressible promoter.

14. A pharmaceutical composition comprising the engineered bacteriophage of claim 1 or the engineered bacteriophage genome polynucleotide of claim 13.

15. A method of treating or preventing a disease in a subject in need thereof, wherein the method comprises administering to the subject the engineered bacteriophage according to claim 1.

16. The method of claim 15, wherein the disease is an infectious disease or cancer.

17. The engineered bacteriophage of claim 4, wherein the heterologous protein is a bacteriophage lysin or a CRISR nuclease.

18. The engineered bacteriophage of claim 5, wherein the antigen is a Gram positive or Gram negative bacterial polypeptide antigen or a Mycobacterium antigen.
